(19) United States
(12) Patent Application Publication
(10) Pub. No.: US 2021/0395210 A1
(43) Pub. Date: Dec. 23, 2021

(54) INHIBITION OF BACTERIAL CHAPERONIN SYSTEMS

(71) Applicants: THE TRUSTEES OF INDIANA UNIVERSITY, Bloomington, IN (US); ARIZONA BOARD OF REGENTS ON BEHALF OF THE UNIVERSITY OF ARIZONA, Tucson, AZ (US)

(72) Inventors: Steven JOHNSON, Indianapolis, IN (US); Eli CHAPMAN, Tucson, AZ (US)

(21) Appl. No.: 17/287,226

(22) PCT Filed: Nov. 1, 2019
(86) PCT No.: PCT/US19/59458

§ 371 Date: Apr. 21, 2021

Related U.S. Application Data
(60) Provisional application No. 62/755,027, filed on Nov. 2, 2018.

(51) Int. Cl. C07D 277/74; C07C 235/56; C07C 233/66; A61P 31/04

(52) U.S. Cl. CPC C07D 277/74; A61P 31/04; C07C 233/66; C07C 235/56

(57) ABSTRACT

The present disclosure relates to novel compounds and methods of killing or inhibiting the growth of bacteria. In some embodiments, a method of killing or inhibiting the growth of bacteria is provided. The method comprises administering a compound of formula I or a pharmaceutically acceptable salt thereof to bacteria. In some embodiments, a method of killing or inhibiting the growth of bacteria is provided. The method comprises administering an anthelmintic to bacteria.

### CROSS-REFERENCE TO RELATED APPLICATIONS

This application claims priority under 35 U.S.C. § 119(e) to U.S. Provisional Application Ser. No. 62/755,027 filed on Nov. 2, 2018, the entire disclosure of which is incorporated herein by reference.

### GOVERNMENT SUPPORT CLAUSE

This invention was made with government support under GM120350 awarded by the National Institutes of Health. The government has certain rights in the invention.

### TECHNICAL FIELD

The present disclosure relates to certain compounds and methods of killing or inhibiting the growth of bacteria. Specifically, the compounds target GroEL/ES chaperonin systems.

### BACKGROUND

While disrupting protein homeostasis has proven an effective antibacterial strategy in the context of inhibiting the assembly of ribosomal or transcriptional machinery, perturbing protein folding pathways has gone largely unexplored. To facilitate newly synthesized polypeptides folding to their active/native structural conformations, cells have evolved a class of accessory proteins termed molecular chaperones (Hartl 2011). Molecular chaperones, also known as Heat Shock Proteins (HSPs), are divided into 5 general classes based on the molecular weights of their subunits: HSP100, HSP90, HSP70, HSP60 chaperonins, and small HSPs (Kumar 2015). When molecular chaperone functions are compromised, non-native polypeptides misfold and aggregate, which is detrimental to cell viability (Stefani 2004; Maisonneuve 2008; Carmichael 2000; Bao 2002). Thus, targeting molecular chaperones with small molecule inhibitors should be an effective strategy for killing bacteria that is unique from the mechanisms of current antibiotics.

While research is underway to target HSP70 and HSP90 chaperones as antibiotic strategies, targeting HSP60 chaperonin systems, called GroEL chaperonins in bacteria, has gone largely unexplored. GroEL functions to refold substrate polypeptides through a mechanism unique from other molecular chaperones. GroEL is a homo-tetradecameric protein that consists of two, seven-membered rings that stack back-to-back with each other. To facilitate the folding of substrate polypeptides, GroEL requires binding of ATP and a co-chaperone, called GroES. GroES binding to the GroEL apical domains encapsulates the unfolded polypeptide, where it can attempt to fold within the ring and is sequestered from the outside environment.

It is now apparent that bacteria have intrinsic mechanisms to evade the effects of antibiotics. For instance, many bacteria can surround themselves in a highly impermeable matrix made up of proteins and polysaccharides, known as biofilm. While vancomycin is effective at treating planktonic (free-floating) Staphylococcus aureus, it cannot penetrate biofilms; thus, S. aureus bacteria are able to hide out within these reservoirs until drugs are systemically cleared (Stewart 2001; Singh 2010). Biofilm formation has been associated with poor prognosis in diseases such as cystic fibrosis, and enhances persistence and spread of infection by adhering to tissues and medical devices (Costerton 1999; Musk Jr. 2006). Continued presence of these biofilms has been a hallmark of cases of chronic infection, demonstrating increased resistance to treatments through time as they persist (Bjarnsholt 2013).

While innate mechanisms predispose some bacteria to being naturally resistant to various classes of antibiotics, a striking observation was noted just a few short years after introduction of the early antibiotics: bacterial strains were identified that were resistant to what were previously effective drug dosages. Scientists began to realize that antibiotic-specific resistance was stemming from two primary mechanisms. In the first mechanism, bacteria were accumulating mutations in their own genes to prevent drugs from binding to their targets. An example of this is resistance to quinolone antibiotics, where bacteria accumulate mutations in topoisomerases including GyrA, GyrB, ParA, and ParC (Eaves 2004). This process raises fitness in cultures exhibiting this genotype, thriving where wild-type (WT) strains do not.

In the second mechanism of acquired resistance, it was found that bacteria can acquire new genes from other bacteria through a process called conjugal transfer (Llosa 2002). For example, strains of S. aureus have become resistant to vancomycin by acquiring the vanA operon from Enterococcus faecalis (Hobbs 1973; Gonzalez-Zorn 2003). In the acquisition of this operon, S. aureus can synthesize peptide intermediates that aren't susceptible to vancomycin. These peptide intermediates can then cross-link forming peptidoglycan, thus continuing growth.

To circumvent pre-disposed resistance mechanisms, there is a need for new antibacterials that function through new mechanisms of action and against previously unexploited pathways.

### SUMMARY

In some embodiments, the disclosure relates to a compound of the formula I

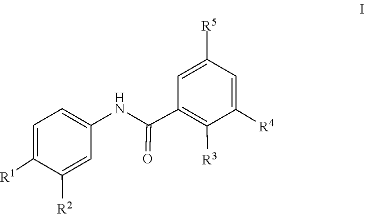

wherein

R¹ is H or

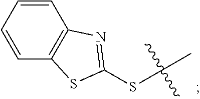 [SMILES: *Sc1nc2ccccc2s1]

R² is a halogen or H;

R³ is H, OH, or OCH₃;

R⁴ is a halogen or H; and

R⁵ is a halogen or H;

provided that at least one of R¹, R², R³, R⁴, or R⁵ is not H;

or a pharmaceutically acceptable salt thereof.

In some embodiments, the compound of formula I, has the formula

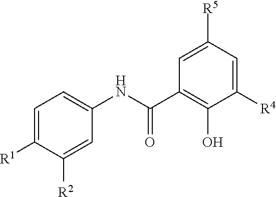

or a pharmaceutically acceptable salt thereof.

In some embodiments, the compound of formula I, has the formula

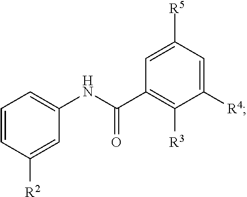

or a pharmaceutically acceptable salt thereof.

In some embodiments, a method of killing or inhibiting the growth of bacteria is provided. The method comprises administering a compound of formula I or a pharmaceutically acceptable salt thereof to bacteria.

In some embodiments, a method of killing or inhibiting the growth of bacteria is provided. The method comprises administering an anthelmintic to bacteria. In some embodiments, the anthelmintic is selected from a group consisting of closantel, rafoxanide, and niclosamide.

### BRIEF DESCRIPTION OF THE DRAWINGS

FIGS. 1A-1B are graphs showing correlation plots of IC₅₀ values for some exemplary compounds evaluated in a GroEL/ES chaperonin biochemical assay, wherein FIG. 1A shows compounds inhibit nearly equipotently in the GroEL/ES-denaturedMDH and the GroEL/ES-denaturedRho refolding assays, and FIG. 1B showing the tested compounds acted on the GroEL/ES system and did not inhibit native Rho (and in most cases native MDH) enzymatic activity, demonstrating specific effects against the GroEL/ES chaperonin system and not the reporter enzymes.

FIGS. 2A-2B are graphs showing correlation plots comparing IC₅₀ values for compounds tested in the GroEL/ES-denaturedMDH refolding assay with the EC₅₀ values for inhibiting proliferation of (FIG. 2A) E. faecium and (FIG. 2B) MRSA. Increasing potencies against bacteria with increasing inhibition of GroEL/ES supports an on-target mechanism of action against the chaperonin systems.

FIGS. 3A-3C are graphs showing correlation plots comparing human HSP60/10-denaturedMDH and GroEL/ES-denaturedMDH refolding assay IC₅₀, human cell viability CC₅₀, and MRSA proliferation EC₅₀, wherein FIG. 3A shows equipotent inhibition between the two refolding assays suggesting the binding sites are potentially conserved, FIG. 3B shows the compounds tested exhibited low to no cytotoxic effects against human liver and kidney cells (a lack of an apparent correlation suggests cytotoxicity is independent of targeting HSP60/10 in human cells), and FIG. 3C shows lead analogs inhibit MRSA proliferation with high selectivity compared to cytotoxicity to human liver and kidney cells (suggesting a high therapeutic window).

FIG. 4 is a graph showing the ability of MRSA to develop resistance to vancomycin and previously identified inhibitor 28R, but retained sensitivity to compounds 1 and 11 over a 12 day period of continual culturing of bacteria.

FIGS. 5A-5B show a comparison of dose-response plots for compound 1 (FIG. 5A) and vancomycin (FIG. 5B) for inhibiting the growth of planktonic S. aureus, the ability of planktonic S. aureus to form biofilms, and eradication of S. aureus in established biofilms.

FIG. 6 shows the crystal structure of GroEL/ES from the side and top view.

FIG. 7 is a cartoon representation of how GroEL/ES functions to fold unfolded polypeptides.

### DETAILED DESCRIPTION

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

Except as otherwise noted, the methods and techniques of the present embodiments are generally performed according to conventional methods well known in the art and as described in various general and more specific references that are cited and discussed throughout the present specification. See, e.g., Loudon, Organic Chemistry, Fourth Edition, New York: Oxford University Press, 2002, pp. 360-361, 1084-1085; Smith and March, March's Advanced Organic Chemistry: Reactions, Mechanisms, and Structure, Fifth Edition, Wiley-Interscience, 2001.

Chemical nomenclature for compounds described herein has generally been derived using the commercially-available ACD/Name 2014 (ACD/Labs) or ChemBioDraw Ultra 13.0 (Perkin Elmer).

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination. All combinations of the embodiments pertaining to the chemical groups represented by the variables are specifically embraced by the present disclosure and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present disclosure and are disclosed herein just as if each and every such sub-combination of chemical groups was individually and explicitly disclosed herein.

### Definitions

As used herein, “halogen” refers to fluorine, chlorine, bromine, or idodine.

As used herein, the term “pharmaceutically acceptable salt” refers to those salts with counter ions which may be used in pharmaceuticals. See, generally, S. M. Berge, et al., “Pharmaceutical Salts,” J. Pharm. Sci., 1977, 66, 1-19. Preferred pharmaceutically acceptable salts are those that are pharmacologically effective and suitable for contact with the tissues of subjects without undue toxicity, irritation, or allergic response. A compound described herein may possess a sufficiently acidic group, a sufficiently basic group, both types of functional groups, or more than one of each type, and accordingly react with a number of inorganic or organic bases, and inorganic and organic acids, to form a pharmaceutically acceptable salt. Such salts include:

(1) acid addition salts, which can be obtained by reaction of the free base of the parent compound with inorganic acids such as hydrochloric acid, hydrobromic acid, nitric acid, phosphoric acid, sulfuric acid, and perchloric acid and the like, or with organic acids such as acetic acid, oxalic acid, (D) or (L) malic acid, maleic acid, methane sulfonic acid, ethanesulfonic acid, p-toluenesulfonic acid, salicylic acid, tartaric acid, citric acid, succinic acid or malonic acid and the like; or

(2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, trimethamine, N-methylglucamine, and the like.

Pharmaceutically acceptable salts are well known to those skilled in the art, and any such pharmaceutically acceptable salt may be contemplated in connection with the embodiments described herein. Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in Remington's Pharmaceutical Sciences, 17th Edition, Mack Publishing Company, Easton, Pa., 1985.

Any formula depicted herein is intended to represent a compound of that structural formula as well as certain variations or forms. For example, a formula given herein is intended to include a racemic form, or one or more enantiomeric, diastereomeric, or geometric isomers, or a mixture thereof. Additionally, any formula given herein is intended to refer also to a hydrate, solvate, or polymorph of such a compound, or a mixture thereof. For example, it will be appreciated that compounds depicted by a structural formula containing the symbol “ [symbol] ” include both stereoisomers for the carbon atom to which the symbol “ [symbol] ” is attached, specifically both the bonds “ [symbol] ” and “ [symbol] ” are encompassed by the meaning of “ [symbol] ”.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine, chlorine, and iodine, such as ²H, ³H, ¹¹C, ¹³C, ¹⁴C, ¹⁵N, ¹⁸O, ¹⁷O ³¹P, ³²P, ³⁵S, ¹⁸F, ³⁶Cl, and ¹²⁵I, respectively. Such isotopically labelled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent.

### Representative Embodiments

In some embodiments, compounds described herein comprise a compound of the formula I

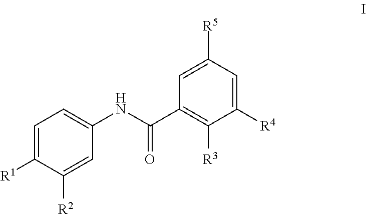

or a pharmaceutically acceptable salt thereof.

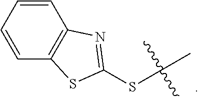 [SMILES: *Sc1nc2ccccc2s1]

In some embodiments R¹ is H or In some embodiments R¹ is H. In some embodiments, R¹ is

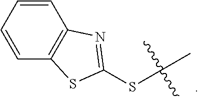 [SMILES: *Sc1nc2ccccc2s1]

In some embodiments, R² is a halogen or H. In some embodiments, R² is a halogen. In some embodiments, R² is H or Cl. In some embodiments, R² is H. In some embodiments, R² is Cl.

In some embodiments, R³ is H, OH, or OCH₃. In some embodiments, R³ is H. In some embodiments, R³ is OH. In some embodiments, R³ is OCH₃. In some embodiments, R³ is H or OH. In some embodiments, R³ is H or OCH₃. In some embodiments, R³ is OH or OCH₃.

In some embodiments, R⁴ is a halogen or H. In some embodiments, R⁴ is a halogen. In some embodiments, R⁴ is H or Br. In some embodiments, R⁴ is H. In some embodiments, R⁴ is Br.

In some embodiments, R⁵ is a halogen or H. In some embodiments, R⁵ is a halogen. In some embodiments, R⁵ is H or Br. In some embodiments, R⁵ is H. In some embodiments, R⁵ is Br.

In some embodiments, at least one of R¹, R², R³, R⁴, or R⁵ is not H. In some embodiments, at least two of of R¹, R², R³, R⁴, and R⁵ are independently not H. In some embodiments, at least three of of R₁, R², R³, R⁴, and R⁵ are independently not H. In some embodiments, at least four of of R″ R², R³, R⁴, and R⁵ are independently not H.

In some embodiments, R⁴ is Br and R⁵ is Br.

In some embodiments, a method of killing or inhibiting the growth of bacteria comprising contacting a compound of formula I or a pharmaceutically acceptable salt to the bacteria is provided. In some embodiments, the bacteria is Gram-positive. In some embodiments, the bacteria is Gram-negative. In some embodiments, the bacteria comprise Gram-positive bacteria, Gram-negative bacteria, or a combination thereof. In some embodiments, the bacteria are capable of forming a biofilm. In some embodiments, the genus of bacteria are selected from a group consisting of Enterococcus, Staphylococcus, Klebsiella, Acinetobacter, Pseudomonas, and Enterobacter or a combination thereof. In some embodiments, the bacteria are Enterococcus faecium, Staphylococcus aureus, methicillin-resistant Staphylococcus aureus (MRSA), Klebsiella pneumoniae, Acinetobacter baumannii, Pseudomonas aeruginosa, Enterobacter cloacae, or a combination thereof.

In some embodiments, a method of killing or inhibiting the growth of bacteria forming a biofilm is provided. The method comprises contacting the bacteria with a compound of formula I or a pharmaceutically acceptable salt thereof.

In some embodiments, a method of killing or inhibiting the growth of bacteria within a biofilm is provided. The method comprises contacting the biofilm with a compound of formula I or a pharmaceutically acceptable salt thereof.

The following represent illustrative embodiments of compounds of the formula I:

| Compound | Structure | Name |
|---|---|---|
| 1 | 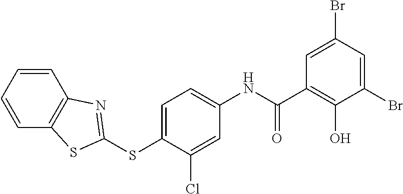 [SMILES: O=C(Nc1ccc(Sc2nc3ccccc3s2)c(Cl)c1)c1cc(Br)cc(Br)c1O] | N-(4-(Benzo[d]thiazol-2-ylthio)-3- chlorophenyl)-3,5-dibromo-2- hydroxybenzamide |
| 2 | 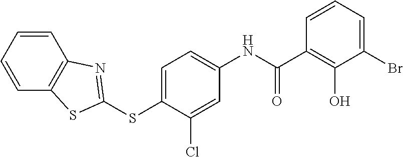 [SMILES: O=C(Nc1ccc(Sc2nc3ccccc3s2)c(Cl)c1)c1cccc(Br)c1O] | N-(4-(Benzo[d]thiazol-2-ylthio)-3- chlorophenyl)-3-bromo-2- hydroxybenzamide |
| 3 | 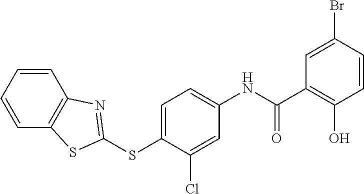 [SMILES: O=C(Nc1ccc(Sc2nc3ccccc3s2)c(Cl)c1)c1cc(Br)ccc1O] | N-(4-(Benzo[d]thiazol-2-ylthio)-3- chlorophenyl)-5-bromo-2- hydroxybenzamide |
| 4 | 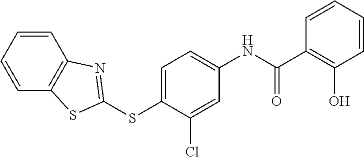 [SMILES: O=C(Nc1ccc(Sc2nc3ccccc3s2)c(Cl)c1)c1ccccc1O] | N-(4-(Benzo[d]thiazol- 2-ylthio)-3- chlorophenyl)-2- hydroxybenzamide |
| 5 | 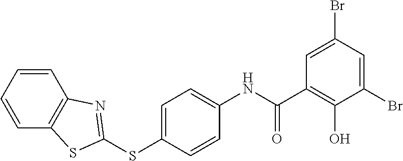 [SMILES: O=C(Nc1ccc(Sc2nc3ccccc3s2)cc1)c1cc(Br)cc(Br)c1O] | N-(4-(Benzo[d]thiazol- 2-ylthio)phenyl)-3,5- dibromo-2-hydroxybenzamide |
| 6 | 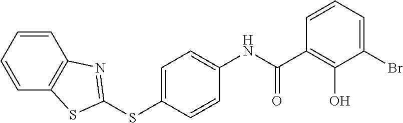 [SMILES: O=C(Nc1ccc(Sc2nc3ccccc3s2)cc1)c1cccc(Br)c1O] | N-(4-(Benzo[d]thiazol- 2-ylthio)phenyl)-3- bromo-2-hydroxybenzamide |
| 7 | 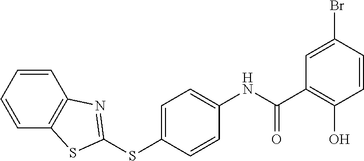 [SMILES: O=C(Nc1ccc(Sc2nc3ccccc3s2)cc1)c1cc(Br)ccc1O] | N-(4-(Benzo[d]thiazol-2- ylthio)phenyl)-5- bromo-2-hydroxybenzamide |
| 8 | 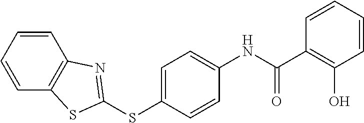 [SMILES: O=C(Nc1ccc(Sc2nc3ccccc3s2)cc1)c1ccccc1O] | N-(4-(Benzo[d]thiazol-2- ylthio)phenyl)-2- hydroxybenzamide |
| 9 | 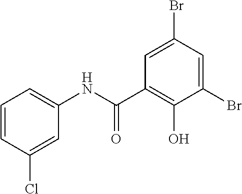 [SMILES: O=C(Nc1cccc(Cl)c1)c1cc(Br)cc(Br)c1O] | 3,5-Dibromo-N-(3-chlorophenyl)-2- hydroxybenzamide |
| 10 | 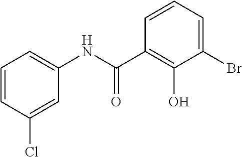 [SMILES: O=C(Nc1cccc(Cl)c1)c1cccc(Br)c1O] | 3-Bromo-N-(3-chlorophenyl)-2- hydroxybenzamide |
| 11 | 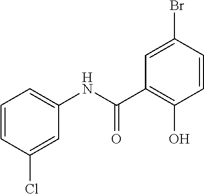 [SMILES: O=C(Nc1cccc(Cl)c1)c1cc(Br)ccc1O] | 5-Bromo-N-(3-chlorophenyl)-2- hydroxybenzamide |
| 12 | 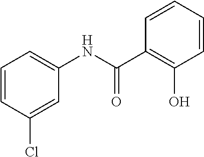 [SMILES: O=C(Nc1cccc(Cl)c1)c1ccccc1O] | N-(3-Chlorophenyl)-2- hydroxybenzamide. |
| 13 | 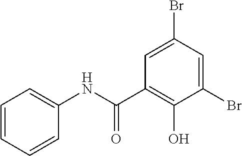 [SMILES: O=C(Nc1ccccc1)c1cc(Br)cc(Br)c1O] | 3,5-Dibromo-2-hydroxy-N- phenylbenzamide |
| 14 | 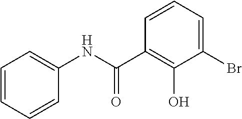 [SMILES: O=C(Nc1ccccc1)c1cccc(Br)c1O] | 3-Bromo-2-hydroxy-N- phenylbenzamide |
| 15 | 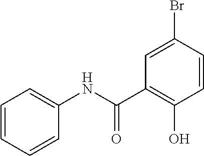 [SMILES: O=C(Nc1ccccc1)c1cc(Br)ccc1O] | 5-Bromo-2-hydroxy-N- phenylbenzamide |
| 16 | 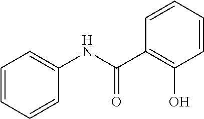 [SMILES: O=C(Nc1ccccc1)c1ccccc1O] | 2-Hydroxy-N-phenylbenzamide |
| 17 | 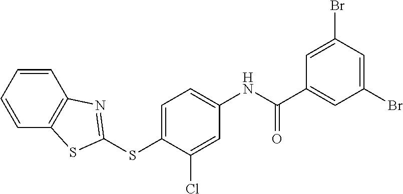 [SMILES: O=C(Nc1ccc(Sc2nc3ccccc3s2)c(Cl)c1)c1cc(Br)cc(Br)c1] | N-(4-(Benzo[d]thiazol-2-ylthio)-3- chlorophenyl)-3,5-dibromobenzamide |
| 18 | 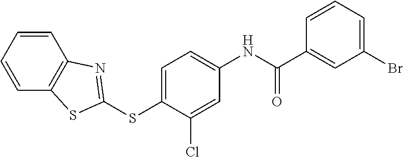 [SMILES: O=C(Nc1ccc(Sc2nc3ccccc3s2)c(Cl)c1)c1cccc(Br)c1] | N-(4-(Benzo[d]thiazol-2-ylthio)-3- chlorophenyl)-3-bromobenzamide |
| 19 | 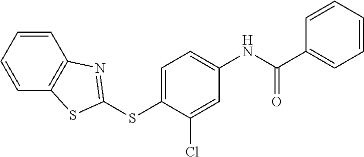 [SMILES: O=C(Nc1ccc(Sc2nc3ccccc3s2)c(Cl)c1)c1ccccc1] | N-(4-(Benzo[d]thiazol-2-ylthio)-3- chlorophenyl)benzamide |
| 20 | 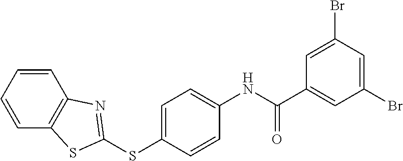 [SMILES: O=C(Nc1ccc(Sc2nc3ccccc3s2)cc1)c1cc(Br)cc(Br)c1] | N-(4-(Benzo[d]thiazol- 2-ylthio)phenyl)-3,5- dibromobenzamide |
| 21 | 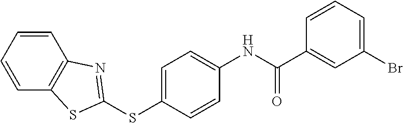 [SMILES: O=C(Nc1ccc(Sc2nc3ccccc3s2)cc1)c1cccc(Br)c1] | N-(4-(Benzo[d]thiazol- 2-ylthio)phenyl)-3- bromobenzamide |
| 22 | 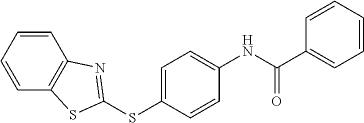 [SMILES: O=C(Nc1ccc(Sc2nc3ccccc3s2)cc1)c1ccccc1] | N-(4-(Benzo[d]thiazol-2- ylthio)phenyl)benzamide |
| 23 | 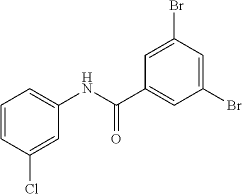 [SMILES: O=C(Nc1cccc(Cl)c1)c1cc(Br)cc(Br)c1] | 3,5-Dibromo-N-(3- chlorophenyl)benzamide |
| 24 | 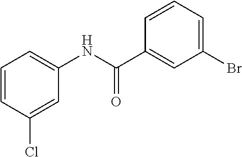 [SMILES: O=C(Nc1cccc(Cl)c1)c1cccc(Br)c1] | 3-Bromo-N-(3- chlorophenyl)benzamide |
| 25 | 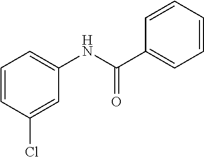 [SMILES: O=C(Nc1cccc(Cl)c1)c1ccccc1] | N-(3-Chlorophenyl)benzamide |
| 26 | 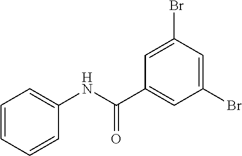 [SMILES: O=C(Nc1ccccc1)c1cc(Br)cc(Br)c1] | 3,5-Dibromo-N-phenylbenzamide |
| 27 | 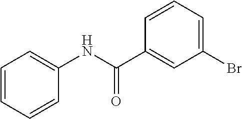 [SMILES: O=C(Nc1ccccc1)c1cccc(Br)c1] | 3-Bromo-N-phenylbenzamide |
| 28 | 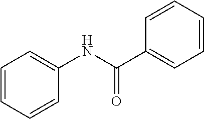 [SMILES: O=C(Nc1ccccc1)c1ccccc1] | N-Phenylbenzamide |
| 29 | 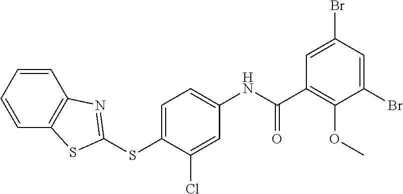 [SMILES: COc1c(Br)cc(Br)cc1C(=O)Nc1ccc(Sc2nc3ccccc3s2)c(Cl)c1] | N-(4-(Benzo[d]thiazol-2-ylthio)-3- chlorophenyl)-3,5-dibromo-2- methoxybenzamide |
| 30 | 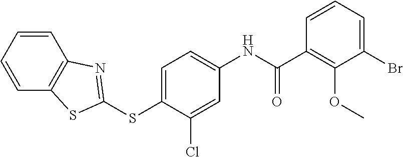 [SMILES: COc1c(Br)cccc1C(=O)Nc1ccc(Sc2nc3ccccc3s2)c(Cl)c1] | N-(4-(Benzo[d]thiazol-2-ylthio)-3- chlorophenyl)-3-bromo-2- methoxybenzamide |
| 31 | 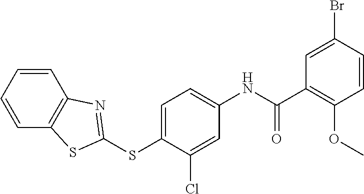 [SMILES: COc1ccc(Br)cc1C(=O)Nc1ccc(Sc2nc3ccccc3s2)c(Cl)c1] | N-(4-(Benzo[d]thiazol-2-ylthio)-3- chlorophenyl)-5-bromo-2- methoxybenzamide |
| 32 | 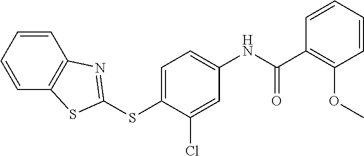 [SMILES: COc1ccccc1C(=O)Nc1ccc(Sc2nc3ccccc3s2)c(Cl)c1] | N-(4-(Benzo[d]thiazol-2-ylthio)-3- chlorophenyl)-2-methoxybenzamide. |
| 33 | 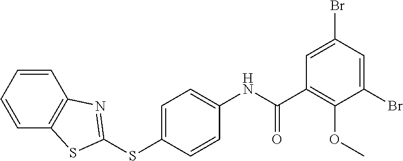 [SMILES: COc1c(Br)cc(Br)cc1C(=O)Nc1ccc(Sc2nc3ccccc3s2)cc1] | N-(4-(Benzo[d]thiazol- 2-ylthio)phenyl)-3,5- dibromo-2-methoxybenzamide |
| 34 | 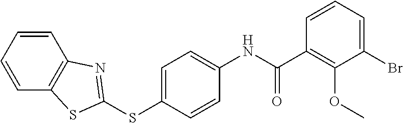 [SMILES: COc1c(Br)cccc1C(=O)Nc1ccc(Sc2nc3ccccc3s2)cc1] | N-(4-(Benzo[d]thiazol- 2-ylthio)phenyl)-3- bromo-2-methoxybenzamide |
| 35 | 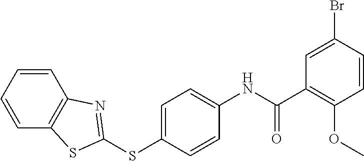 [SMILES: COc1ccc(Br)cc1C(=O)Nc1ccc(Sc2nc3ccccc3s2)cc1] | N-(4-(Benzo[d]thiazol- 2-ylthio)phenyl)-5- bromo-2-methoxybenzamide |
| 36 | 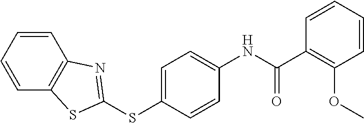 [SMILES: COc1ccccc1C(=O)Nc1ccc(Sc2nc3ccccc3s2)cc1] | N-(4-(Benzo[d]thiazol- 2-ylthio)phenyl)-2- methoxybenzamide |
| 37 | 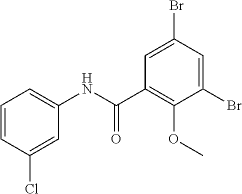 [SMILES: COc1c(Br)cc(Br)cc1C(=O)Nc1cccc(Cl)c1] | 3,5-Dibromo-N- (3-chlorophenyl)-2- methoxybenzamide |
| 38 | 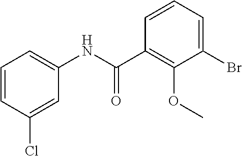 [SMILES: COc1c(Br)cccc1C(=O)Nc1cccc(Cl)c1] | 3,5-Dibromo-N-(3-chlorophenyl)-2- methoxybenzamide |
| 39 | 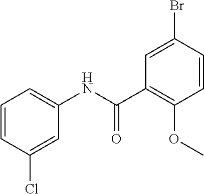 [SMILES: COc1ccc(Br)cc1C(=O)Nc1cccc(Cl)c1] | 5-Bromo-N-(3-chlorophenyl)-2- methoxybenzamide |
| 40 | 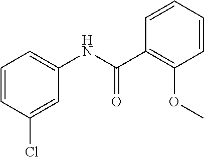 [SMILES: COc1ccccc1C(=O)Nc1cccc(Cl)c1] | N-(3-Chlorophenyl)-2- methoxybenzamide |
| 41 | 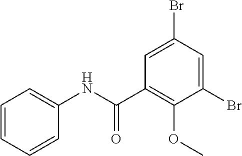 [SMILES: COc1c(Br)cc(Br)cc1C(=O)Nc1ccccc1] | 3,5-Dibromo-2-methoxy-N- phenylbenzamide |
| 42 | 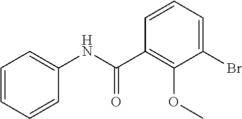 [SMILES: COc1c(Br)cccc1C(=O)Nc1ccccc1] | 3-Bromo-2-methoxy-N- phenylbenzamide |
| 43 | 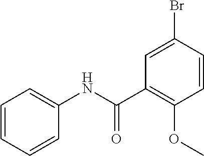 [SMILES: COc1ccc(Br)cc1C(=O)Nc1ccccc1] | 5-Bromo-2-methoxy-N- phenylbenzamide |
| 44 | 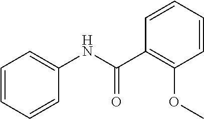 [SMILES: COc1ccccc1C(=O)Nc1ccccc1] | 2-Methoxy-N- phenylbenzamide |

Clause 1. A compound of the formula I

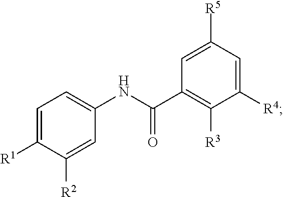

wherein
R¹ is H or

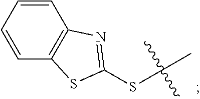 [SMILES: *Sc1nc2ccccc2s1]

R² is a halogen or H;
R³ is H, OH, or OCH₃;
R⁴ is a halogen or H; and
R⁵ is a halogen or H;
provided that at least one of R¹, R², R³, R⁴, or R⁵ is not H;
or a pharmaceutically acceptable salt thereof.

Clause 2. The compound or pharmaceutically acceptable salt of clause 1, having the formula

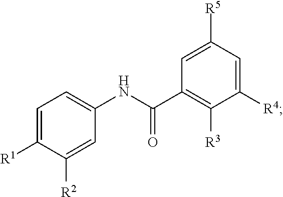

or a pharmaceutically acceptable salt thereof.

Clause 3. The compound or pharmaceutically acceptable salt of clause 1 or 2, wherein R¹ is H.

Clause 4. The compound or pharmaceutically acceptable salt of clause 1 or 2, wherein R¹ is

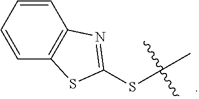 [SMILES: *Sc1nc2ccccc2s1]

Clause 5. The compound or pharmaceutically acceptable salt of any of the preceding clauses, wherein R² is H.

Clause 6. The compound or pharmaceutically acceptable salt of any of the preceding clauses, wherein R² is Cl.

Clause 7. The compound or pharmaceutically acceptable salt of any of the preceding clauses, wherein R⁴ is H.

Clause 8. The compound or pharmaceutically acceptable salt of any of the preceding clauses, wherein R⁴ is Br.

Clause 9. The compound or pharmaceutically acceptable salt of any of the preceding clauses, wherein R⁵ is H.

Clause 10. The compound or pharmaceutically acceptable salt of any of the preceding clauses, wherein R⁵ is Br.

Clause 11. The compound or pharmaceutically acceptable salt of any of the preceding clauses, wherein R⁴ is Br and R⁵ is Br.

Clause 12. The compound or pharmaceutically acceptable salt of clause 11, wherein R¹ is

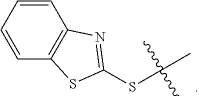 [SMILES: *Sc1nc2ccccc2s1]

Clause 13. The compound or pharmaceutically acceptable salt of clause 12, wherein R² is Cl.

Clause 14. The compound or pharmaceutically acceptable salt of clause 12, wherein R² is H.

Clause 15. The compound or pharmaceutically acceptable salt of clause 1, having the formula

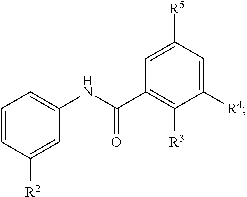

or a pharmaceutically acceptable salt thereof.

Clause 16. A method of killing or inhibiting the growth of bacteria comprising: contacting the bacteria with a compound of formula I

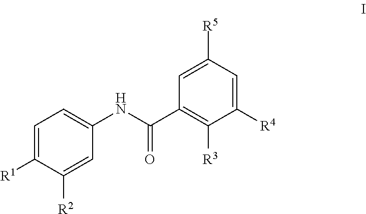

wherein

R¹ is H or

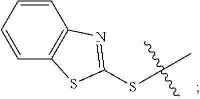 [SMILES: *Sc1nc2ccccc2s1]

R² is a halogen or H;

R³ is H, OH, or OCH₃;

R⁴ is a halogen or H; and

R⁵ is a halogen or H;

provided that at least one of R¹, R², R³, R⁴, or R⁵ is not H;

or a pharmaceutically acceptable salt thereof.

Clause 17. The method of clause 16, having the formula

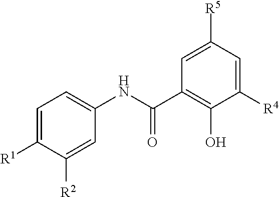

or a pharmaceutically acceptable salt thereof.

Clause 18. The method of clause 16 or 17, wherein R¹ is H.

Clause 19. The method of clause 16 or 17, wherein R¹ is

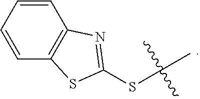 [SMILES: *Sc1nc2ccccc2s1]

Clause 20. The method of any of clauses 16-19, wherein R² is H.

Clause 21. The method of any of clauses 16-19, wherein R² is Cl.

Clause 22. The method of any of clauses 16-21, wherein R⁴ is H.

Clause 23. The method of any of clauses 16-19 or 22, wherein R⁴ is Br.

Clause 24. The method of any of clauses 16-23, wherein R⁵ is H.

Clause 25. The method of any of clauses 16-23, wherein R⁵ is Br.

Clause 26. The method of clause 16 or 17 wherein R⁴ is Br and R⁵ is Br.

Clause 27. The method of clause 16, wherein R¹ is

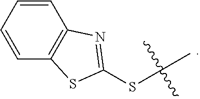 [SMILES: *Sc1nc2ccccc2s1]

Clause 28. The method of clause 27, wherein R² is Cl.

Clause 29. The method of clause 27, wherein R² is H.

Clause 30. A method of killing or inhibiting the growth of bacteria comprising contacting the bacteria with an anthelmintic.

Clause 31. The method of clause 30, wherein the anthelmintic is selected from a group consisting of closantel, rafoxanide, and niclosamide.

Clause 32. A pharmaceutical composition comprising a therapeutically effective amount of a compound according to any one of clauses 1-15.

Clause 33. A compound selected from the group consisting of

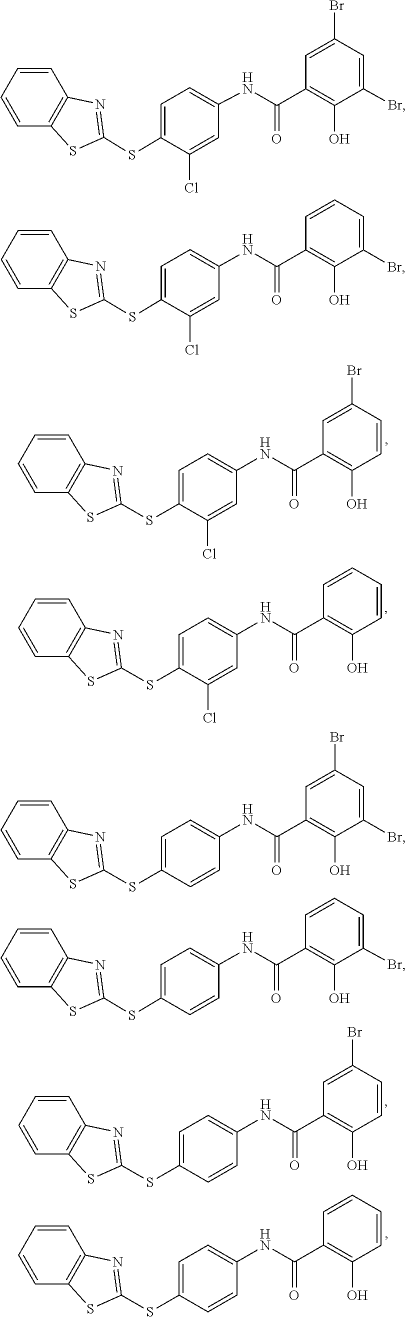

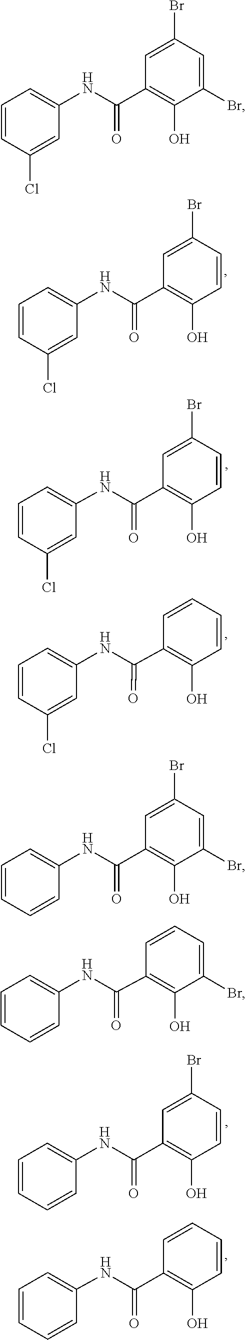

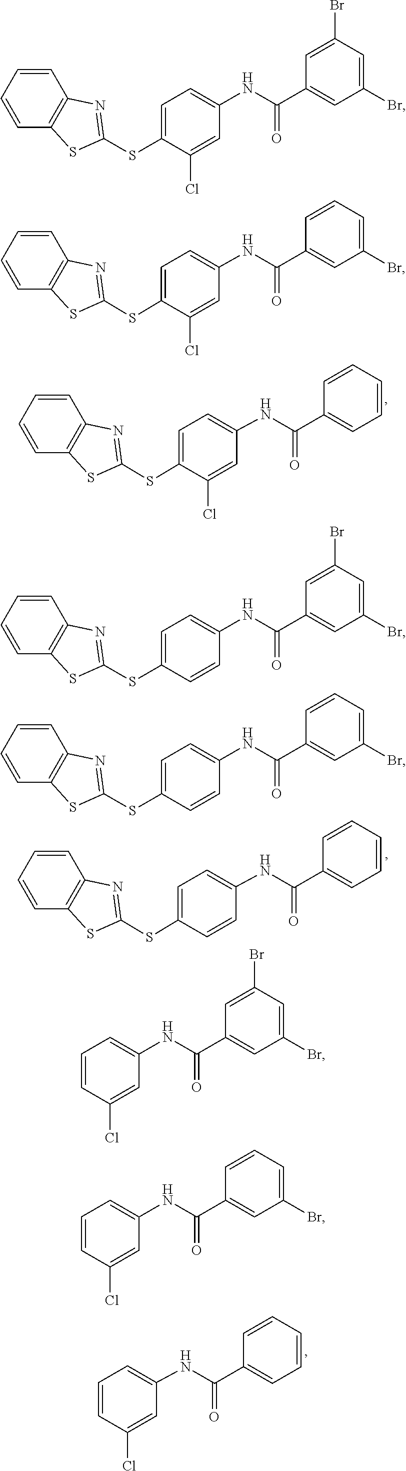

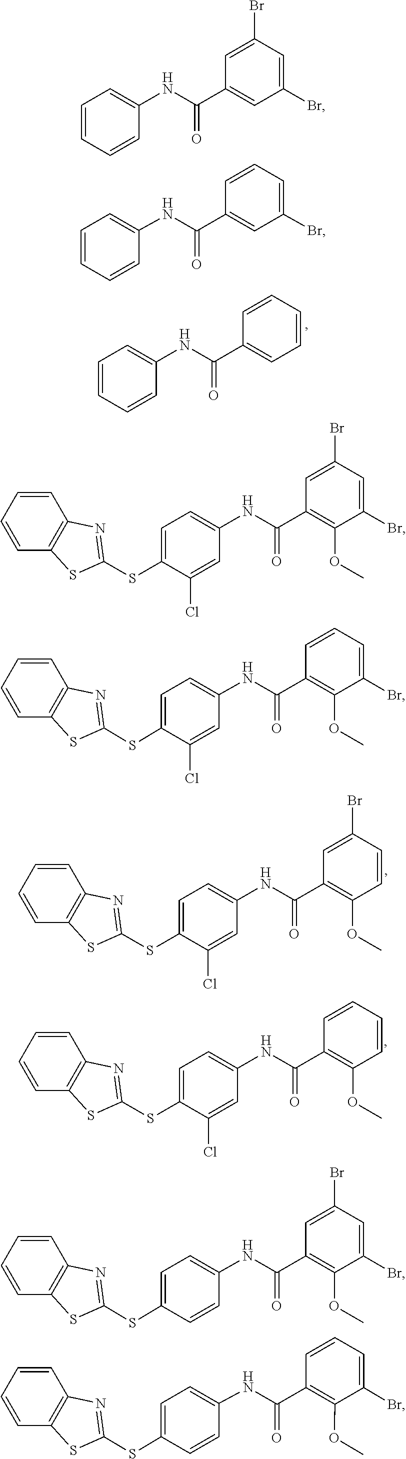

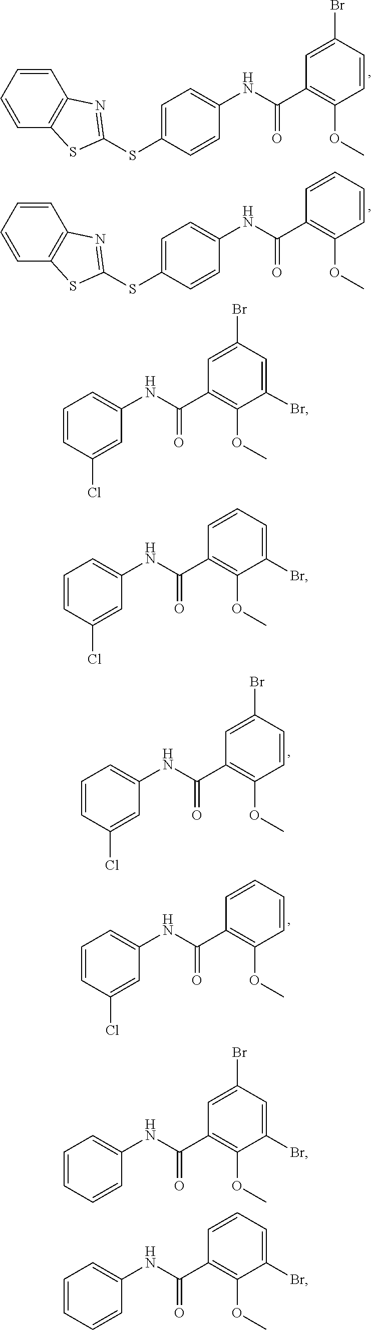

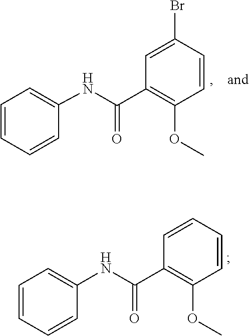 [SMILES: COc1ccc(Br)cc1C(=O)Nc1ccccc1 ; COc1ccccc1C(=O)Nc1ccccc1]

or a pharmaceutically acceptable salt thereof.

Clause 34. A compound of clause 33 selected from the group consisting of

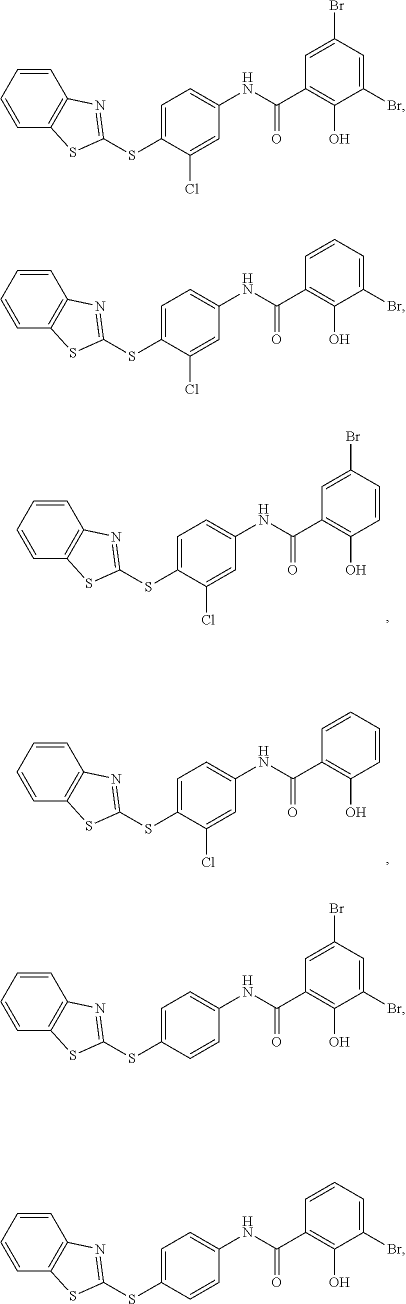 [SMILES: O=C(Nc1ccc(Sc2nc3ccccc3s2)c(Cl)c1)c1cc(Br)cc(Br)c1O ; O=C(Nc1ccc(Sc2nc3ccccc3s2)c(Cl)c1)c1cccc(Br)c1O ; O=C(Nc1ccc(Sc2nc3ccccc3s2)c(Cl)c1)c1cc(Br)ccc1O ; O=C(Nc1ccc(Sc2nc3ccccc3s2)c(Cl)c1)c1ccccc1O ; O=C(Nc1ccc(Sc2nc3ccccc3s2)cc1)c1cc(Br)cc(Br)c1O ; O=C(Nc1ccc(Sc2nc3ccccc3s2)cc1)c1cccc(Br)c1O]

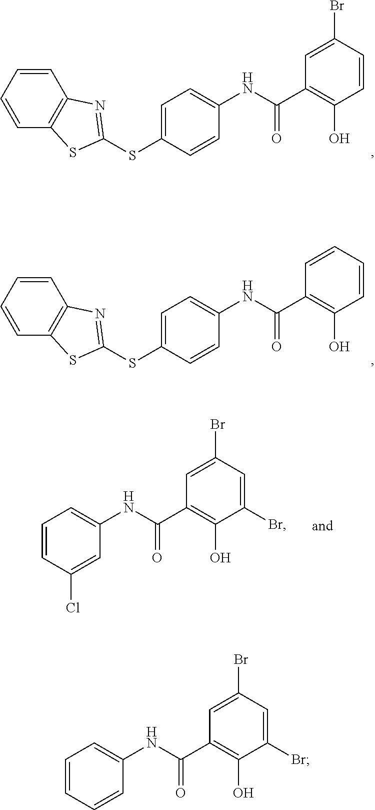 [SMILES: O=C(Nc1ccc(Sc2nc3ccccc3s2)cc1)c1cc(Br)ccc1O ; O=C(Nc1ccc(Sc2nc3ccccc3s2)cc1)c1ccccc1O ; O=C(Nc1cccc(Cl)c1)c1cc(Br)cc(Br)c1O ; O=C(Nc1ccccc1)c1cc(Br)cc(Br)c1O]

or a pharmaceutically acceptable salt thereof.

Clause 35. A method of killing bacteria in a biofilm comprising contacting a compound of any of the preceding claims to the biofilm.

Clause 36. A method of preventing bacteria from forming a biofilm comprising contacting the bacteria with a compound of any of the preceding claims.

Clause 37. The method of clauses 35 and 36, wherein the bacteria is from the genus Staphylococcus.

Clause 38. The compounds of clause 1 or method of claim 16 wherein at least two of R¹, R², R³, R⁴, and R⁵ are independently not H.

Clause 39. The compounds of clauses 1 or method of claim 16 wherein at least three of R¹, R², R³, R⁴, and R⁵ are independently not H.

Clause 40. The compounds of clauses 1 or method of claim 16 wherein at least four of R¹, R², R³, R⁴, and R⁵ are independently not H.

Those skilled in the art will recognize that the species listed or illustrated herein are not exhaustive, and that additional species within the scope of these defined terms may also be selected.

### Chemical Synthesis

Exemplary chemical entities useful in methods of the description will now be described by reference to illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Furthermore, one of skill in the art will recognize that the transformations shown in the schemes below may be performed in any order that is compatible with the functionality of the particular pendant groups.

Abbreviations: The examples described herein use materials, including but not limited to, those described by the following abbreviations known to those skilled in the art:

| | |
|---|---|
| g | grams |
| mmol | millimoles |
| mL | milliliters |
| EtOAc | ethyl acetate |
| MHz | megahertz |
| ppm | parts per million |
| δ | chemical shift |
| s | singlet |
| d | doublet |
| t | triplet |
| q | quartet |
| quin | quintet |
| br | broad |
| m | multiplet |
| Hz | hertz |
| THF | tetrahydrofuran |
| ° C. | degrees Celsius |
| EA | ethyl acetate |
| J | coupling constant |
| d₆₋DMSO | deuterated dimethyl sulfoxide |
| min | minutes |
| hr (or h) | hours |
| Me | methyl |
| Et | ethyl |
| M | molar |
| MS | mass spectrometry |
| m/z | mass-to-charge ratio |
| TFA | trifluoroacetic acid |
| DMAP | 4-(dimethylamino)pyridine |
| μM | micromolar |
| ATP | adenosine triphosphate |
| DCM | dichloromethane |
| DMF | N,N-dimethylformamide |
| TEA | Triethylamine |
| MDH | Malate dehydrogenase |
| Rho | Rhodanese |
| R.T. | Room temperature |
| IC₅₀ | Inhibitory concentration for half-maximal signal in |
| | biochemical assay |
| EC₅₀ | Effective concentration for half-maximal signal in bacterial |
| | proliferation assays |
| CC₅₀ | Cytotoxicity concentration for half-maximal signal in human |
| | cell viability assays. |

### Example 1

General Synthetic Method. Unless otherwise stated, all chemicals were purchased from commercial suppliers and used without further purification. Reaction progress was monitored by thin-layer chromatography on silica gel 60 F254 coated glass plates (EM Sciences). Flash chromatography was performed using a Biotage Isolera One flash chromatography system with elution through Biotage KP-Sil Zip or Snap silica gel columns for normal-phase separations (hexanes:EtOAc gradients) or Snap KP-C18-HS columns for reverse-phase separations (H₂O:MeOH gradients). Reverse-phase high-performance liquid chromatography (RP-HPLC) was performed using a Waters 1525 binary pump, 2489 tunable UV/Vis detector (254 and 280 nm detection), and 2707 autosampler. For preparatory HPLC purification, samples were chromatographically separated using a Waters XSelect CSH C18 OBD prep column (part number 186005422, 130 Å pore size, 5 μm particle size, 19×150 mm), eluting with a H₂O:CH3CN gradient solvent system. Linear gradients were run from either 100:0, 80:20, or 60:40 A:B to 0:100 A:B (A=95:5 H₂O:CH₃CN, 0.05% TFA; B=5:95 H₂O:CH₃CN, 0.05% TFA). Products from normal-phase separations were concentrated directly, and reverse-phase separations were concentrated, diluted with H₂O, frozen, and lyophilized. For primary compound purity analyses (HPLC-1), samples were chromatographically separated using a Waters XSelect CSH C18 column (part number 186005282, 130 Å pore size, 5 μm particle size, 3.0×150 mm), eluting with the above H₂O:CH₃CN gradient solvent systems. For secondary purity analyses (HPLC-2) of final test compounds, samples were chromatographically separated using a Waters XBridge C18 column (either part number 186003027, 130 Å pore size, 3.5 μm particle size, 3.0×100 mm, or part number 186003132, 130 Å pore size, 5.0 μm particle size, 3.0×100 mm), eluting with a H₂O:MeOH gradient solvent system. Linear gradients were run from either 100:0, 80:20, 60:40, or 20:80 A:B to 0:100 A:B (A=95:5 H₂O:MeOH, 0.05% TFA; B=5:95 H₂O:MeOH, 0.05% TFA). Test compounds were found to be >95% pure from both RP-HPLC analyses. Mass spectrometry data were collected using either an Agilent analytical LC-MS at the IU Chemical Genomics Core Facility (CGCF), or a Thermo-Finnigan LTQ LC-MS in-lab. ¹H-NMR spectra were recorded on a Bruker 300 MHz spectrometer at the CGCF. Chemical shifts are reported in parts per million and calibrated to the d₆-DMSO solvent peaks at 2.50 ppm. Synthesis and characterization of intermediates 45-49 are presented below. General amide coupling and methoxy deprotection steps are presented as follows using compounds 29 and 1 as representative examples. Specific synthetic procedures and compound characterizations are presented next for the remaining analogs.

### Example 2

Analog 1: Preparation of N-(4-(Benzo[d]thiazol-2-ylthio)-3-chlorophenyl)-3,5-dibromo-2-hydroxybenzamide. To a stirring mixture of 29 (51.0 mg, 0.0872 mmol) in anhydrous DCM (5 mL) was added BBr₃ (0.26 mL of 1 M in DCM, 0.26 mmol). The reaction was allowed to stir at R.T. (under Ar) for 18 h and then quenched with MeOH. Flash chromatographic purification (hexanes:EtOAc gradient) afforded 1 as an off-white solid (34.4 mg, 69% yield). ¹H-NMR (300 MHz, d₆-DMSO) δ 11.14 (br s, 1H), 8.19 (dd, J=6.2, 2.2 Hz, 2H), 7.94-8.05 (m, 3H), 7.82-7.89 (m, 2H), 7.47 (td, J=7.7, 1.3 Hz, 1H), 7.33-7.40 (m, 1H); MS (ESI) C₂₀H₁₀Br₂ClN₂O₂S₂ [M-H]⁻ m/z expected=566.8, observed=566.6; HPLC-1=99%; HPLC-2=98%.

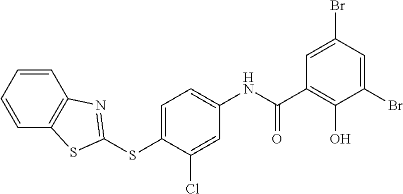 [SMILES: O=C(Nc1ccc(Sc2nc3ccccc3s2)c(Cl)c1)c1cc(Br)cc(Br)c1O]

### Example 3

Analog 2: Preparation of N-(4-(Benzo[d]thiazol-2-ylthio)-3-chlorophenyl)-3-bromo-2-hydroxybenzamide. To a stirring mixture of 30 (70.4 mg, 0.139 mmol) in anhydrous DCM (5 mL) was added BBr₃ (0.42 mL of 1 M in DCM, 0.42 mmol). The reaction was allowed to stir at R.T. (under Ar) for 18 h and then quenched with MeOH. Flash chromatographic purification (hexanes:EtOAc gradient) afforded 2 as an off-white solid (53.0 mg, 77% yield). ¹H-NMR (300 MHz, d₆-DMSO) δ 12.19 (br s, 1H), 10.93 (s, 1H), 8.22 (d, J=2.1 Hz, 1H), 7.94-8.03 (m, 3H), 7.81-7.90 (m, 3H), 7.47 (t, J=7.6 Hz, 1H), 7.33-7.40 (m, 1H), 6.99 (t, J=7.9 Hz, 1H); MS (ESI) C₂₀H₁₁BrClN₂O₂S₂ [M-H]⁻ m/z expected=488.9, observed=488.7; HPLC-1=99%; HPLC-2=99%.

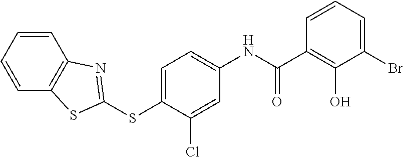 [SMILES: O=C(Nc1ccc(Sc2nc3ccccc3s2)c(Cl)c1)c1cccc(Br)c1O]

### Example 4

Analog 3: Preparation of N-(4-(Benzo[d]thiazol-2-ylthio)-3-chlorophenyl)-5-bromo-2-hydroxybenzamide. To a stirring mixture of 31 (69.0 mg, 0.136 mmol) in anhydrous DCM (5 mL) was added BBr₃ (0.41 mL of 1 M in DCM, 0.41 mmol). The reaction was allowed to stir at R.T. (under Ar) for 18 h and then quenched with MeOH. Flash chromatographic purification (hexanes:EtOAc gradient) afforded 3 as an off-white solid (57.4 mg, 86% yield). ¹H-NMR (300 MHz, d₆-DMSO) δ 11.47 (br s, 1H), 10.74 (s, 1H), 8.25 (d, J=2.2 Hz, 1H), 7.93-8.01 (m, 3H), 7.82-7.89 (m, 2H), 7.60 (dd, J=8.8, 2.6 Hz, 1H), 7.47 (t, J=7.1 Hz, 1H), 7.31-7.39 (m, 1H), 6.99 (d, J=8.8 Hz, 1H); MS (ESI) C₂₀H₁₁BrClN₂O₂S₂ [M-H]⁻ m/z expected=488.9, observed=488.7; HPLC-1=99%; HPLC-2=98%.

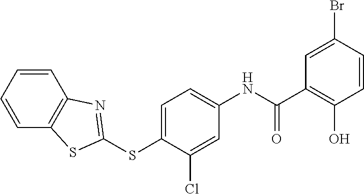 [SMILES: O=C(Nc1ccc(Sc2nc3ccccc3s2)c(Cl)c1)c1cc(Br)ccc1O]

### Example 5

Analog 4: Preparation of N-(4-(Benzo[d]thiazol-2-ylthio)-3-chlorophenyl)-2-hydroxybenzamide. To a stirring mixture 32 (151 mg, 0.353 mmol) in anhydrous DCM (5 mL) was added BBr₃ (1.06 mL of 1 M in DCM, 1.06 mmol). The reaction was allowed to stir at R.T. (under Ar) for 18 h and then quenched with MeOH. Flash chromatographic purification (hexanes:EtOAc gradient), followed by preparatory RP-HPLC purification, afforded 4 as an off-white solid (100 mg, 69% yield). ¹H-NMR (300 MHz, d₆-DMSO) δ 11.35 (s, 1H), 10.71 (s, 1H), 8.28 (d, J=2.1 Hz, 1H), 7.93-8.00 (m, 2H), 7.83-7.90 (m, 3H), 7.72-7.50 (m, 2H), 7.32-7.38 (m, 1H), 6.95-7.05 (m, 2H); MS (ESI) C₂₀H₁₂ClN₂O₂S₂ [M-H]⁻ m/z expected=411.0, observed=410.9; HPLC-1=99%; HPLC-2=>99%.

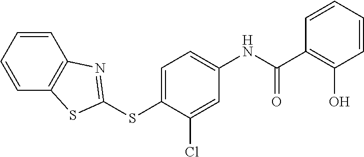 [SMILES: O=C(Nc1ccc(Sc2nc3ccccc3s2)c(Cl)c1)c1ccccc1O]

### Example 6

Analog 5: Preparation of N-(4-(Benzo[d]thiazol-2-ylthio)phenyl)-3,5-dibromo-2-hydroxybenzamide. To a stirring mixture 33 (123 mg, 0.223 mmol) in anhydrous DCM (5 mL) was added BBr₃ (0.67 mL of 1 M in DCM, 0.67 mmol). The reaction was allowed to stir at R.T. (under Ar) for 18 h and then quenched with MeOH. Flash chromatographic purification (hexanes:EtOAc gradient), followed by preparatory RP-HPLC purification, afforded 5 as a white solid (72.9 mg, 61% yield). ¹H-NMR (300 MHz, d₆-DMSO) δ 12.25-13.00 (br s, 1H), 10.94 (s, 1H), 8.25 (d, J=2.3 Hz, 1H), 8.04 (d, J=2.2 Hz, 1H), 7.88-7.96 (m, 3H), 7.80-7.87 (m, 3H), 7.42-7.48 (m, 1H), 7.30-7.37 (m, 1H); MS (ESI) C₂₀H₁₁Br₂N₂O₂S₂[M-H]⁻ m/z expected=532.9, observed=532.7; HPLC-1=97%; HPLC-2=>99%.

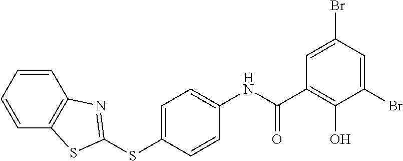 [SMILES: O=C(Nc1ccc(Sc2nc3ccccc3s2)cc1)c1cc(Br)cc(Br)c1O]

### Example 7

Analog 6: Preparation of N-(4-(Benzo[d]thiazol-2-ylthio)phenyl)-3-bromo-2-hydroxybenzamide. To a stirring mixture 34 (108 mg, 0.228 mmol) in anhydrous DCM (5 mL) was added BBr₃ (0.68 mL of 1 M in DCM, 0.68 mmol). The reaction was allowed to stir at R.T. (under Ar) for 18 h and then quenched with MeOH. Flash chromatographic purification (hexanes:EtOAc gradient), followed by preparatory RP-HPLC purification, afforded 6 as a tan solid (55.5 mg, 53% yield). ¹H-NMR (300 MHz, d₆-DMSO) δ 12.40-12.65 (br s, 1H), 10.96 (s, 1H), 8.05 (dd, J=8.0, 1.4 Hz, 1H), 7.90-7.98 (m, 3H), 7.79-7.88 (m, 4H), 7.45 (td, J=7.7, 1.3 Hz, 1H), 7.30-7.37 (m, 1H), 6.96 (t, J=7.9 Hz, 1H); MS (ESI) C₂₀H₁₂BrN₂O₂S₂ [M-H]⁻ m/z expected=455.0, observed=454.8; HPLC-1=>99%; HPLC-2=>99%.

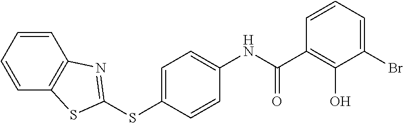 [SMILES: O=C(Nc1ccc(Sc2nc3ccccc3s2)cc1)c1cccc(Br)c1O]

### Example 8

Analog 7: Preparation of N-(4-(Benzo[d]thiazol-2-ylthio)phenyl)-5-bromo-2-hydroxybenzamide. To a stirring mixture 35 (90.6 mg, 0.192 mmol) in anhydrous DCM (5 mL) was added BBr₃ (0.58 mL of 1 M in DCM, 0.58 mmol). The reaction was allowed to stir at R.T. (under Ar) for 18 h and then quenched with MeOH. Flash chromatographic purification (hexanes:EtOAc gradient), followed by preparatory RP-HPLC purification, afforded 7 as a white solid (24.8 mg, 28% yield). ¹H-NMR (300 MHz, d₆-DMSO) δ 11.56-11.66 (br s, 1H), 10.65 (s, 1H), 8.00 (d, J=2.5 Hz, 1H), 7.90-7.98 (m, 3H), 7.78-7.87 (m, 3H), 7.59 (dd, J=8.8, 2.5 Hz, 1H), 7.41-7.48 (m, 1H), 7.30-7.37 (m, 1H), 6.99 (d, J=8.8 Hz, 1H); MS (ESI) C₂₀H₁₂BrN₂O₂S₂ [M-H]⁻ m/z expected=455.0, observed=454.8; HPLC-1=99%; HPLC-2=>99%.

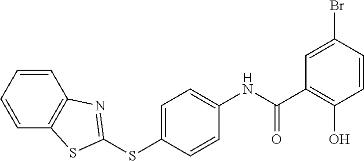 [SMILES: O=C(Nc1ccc(Sc2nc3ccccc3s2)cc1)c1cc(Br)ccc1O]

### Example 9

Analog 8: Preparation of N-(4-(Benzo[d]thiazol-2-ylthio)phenyl)-2-hydroxybenzamide. To a stirring mixture 36 (150 mg, 0.382 mmol) in anhydrous DCM (5 mL) was added BBr₃ (1.15 mL of 1 M in DCM, 1.15 mmol). The reaction was allowed to stir at R.T. (under Ar) for 18 h and then quenched with MeOH. Flash chromatographic purification (hexanes:EtOAc gradient), followed by preparatory RP-HPLC purification, afforded 8 as a white solid (97.3 mg, 67% yield). ¹H-NMR (300 MHz, d₆-DMSO) δ 11.47-11.60 (br s, 1H), 10.64 (s, 1H), 7.90-7.98 (m, 4H), 7.78-7.87 (m, 3H), 7.41-7.49 (m, 2H), 7.29-7.36 (m, 1H), 6.95-7.04 (m, 2H); MS (ESI) C₂₀H₁₃N₂O₂S₂ [M-H]⁻ m/z expected=377.0, observed=376.9; HPLC-1=>99%; HPLC-2=>99%.

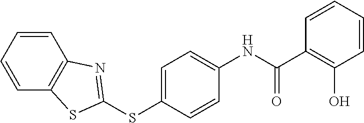 [SMILES: O=C(Nc1ccc(Sc2nc3ccccc3s2)cc1)c1ccccc1O]

### Example 10

Analog 9: Preparation of 3,5-Dibromo-N-(3-chlorophenyl)-2-hydroxybenzamide. To a stirring mixture 37 (200 mg, 0.476 mmol) in anhydrous DCM (5 mL) was added BBr₃ (1.45 mL of 1 M in DCM, 1.45 mmol). The reaction was allowed to stir at R.T. (under Ar) for 3 days and then quenched with MeOH. The product was extracted into EtOAc and the organics were rinsed with brine, dried over Na₂SO₄, filtered, and concentrated. Flash chromatographic purification (hexanes:EtOAc gradient) afforded 9 as a tan solid (128 mg, 66% yield). ¹H-NMR (300 MHz, d₆-DMSO) δ 12.35-12.80 (br s, 1H), 10.74 (s, 1H), 8.24 (d, J=2.2 Hz, 1H), 8.03 (d, J=2.2 Hz, 1H), 7.85 (t, J=1.9 Hz, 1H), 7.60-7.67 (m, 1H), 7.43 (t, J=8.1 Hz, 1H), 7.23-7.29 (m, 1H); MS (ESI) C₁₃H₇Br₂ClNO₂ [M-H]⁻ m/z expected=401.9, observed=401.7; HPLC-1=97%; HPLC-2=97%.

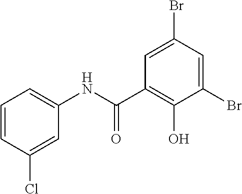 [SMILES: O=C(Nc1cccc(Cl)c1)c1cc(Br)cc(Br)c1O]

### Example 11

Analog 10: Preparation of 3-Bromo-N-(3-chlorophenyl)-2-hydroxybenzamide. To a stirring mixture 38 (155 mg, 0.455 mmol) in anhydrous DCM (5 mL) was added BBr₃ (1.35 mL of 1 M in DCM, 1.35 mmol). The reaction was allowed to stir at R.T. (under Ar) for 18 h and then quenched with MeOH. Reverse-phase flash chromatographic purification (H₂O:MeOH gradient) afforded 10 as a tan solid (138 mg, 93% yield). ¹H-NMR (300 MHz, d₆-DMSO) δ 12.60 (br s, 1H), 10.69 (s, 1H), 8.02 (dd, J=8.0, 1.5 Hz, 1H), 7.87 (t, J=2.0 Hz, 1H), 7.82 (dd, J=7.9, 1.4 Hz, 1H), 7.65 (ddd, J=8.2, 2.0, 0.9 Hz, 1H), 7.43 (t, J=8.1 Hz, 1H), 7.25 (ddd, J=8.0, 2.1, 0.9 Hz, 1H), 6.96 (t, J=7.9 Hz, 1H); MS (ESI) C₁₃H₁₀BrClNO₂ [MH]+m/z expected=327.96, observed=328.10; HPLC-1=>99%; HPLC-2=>99%.

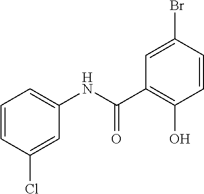 [SMILES: O=C(Nc1cccc(Cl)c1)c1cc(Br)ccc1O]

### Example 12

Analog 11: Preparation of 5-Bromo-N-(3-chlorophenyl)-2-hydroxybenzamide. To a stirring mixture 39 (205 mg, 0.602 mmol) in anhydrous DCM (5 mL) was added BBr₃ (1.80 mL of 1 M in DCM, 1.80 mmol). The reaction was allowed to stir at R.T. (under Ar) for 18 h and then quenched with MeOH. Reverse-phase flash chromatographic purification (H₂O:MeOH gradient) afforded 11 as a white solid (113 mg, 58% yield). ¹H-NMR (300 MHz, d₆-DMSO) δ 11.65 (br s, 1H), 10.50 (s, 1H), 8.00 (d, J=2.5 Hz, 1H), 7.91 (t, J=2.0 Hz, 1H), 7.55-7.64 (m, 2H), 7.40 (t, J=8.1 Hz, 1H), 7.20 (ddd, J=8.0, 2.0, 0.9 Hz, 1H), 6.97 (d, J=8.8 Hz, 1H); MS (ESI) C₁₃H₁₀BrClNO₂ [MH]+m/z expected=327.96, observed=328.10; HPLC-1=>99%; HPLC-2=>99%.

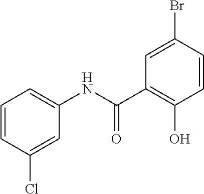 [SMILES: O=C(Nc1cccc(Cl)c1)c1cc(Br)ccc1O]

### Example 13

Analog 12: Preparation of N-(3-Chlorophenyl)-2-hydroxybenzamide. To a stirring mixture 40 (142 mg, 0.543 mmol) in anhydrous DCM (5 mL) was added BBr₃ (1.65 mL of 1 M in DCM, 1.65 mmol). The reaction was allowed to stir at R.T. (under Ar) for 18 h and then quenched with MeOH. Reverse-phase flash chromatographic purification (H₂O:MeOH gradient) afforded 12 as an off-white solid (142 mg, 70% yield). ¹H-NMR (300 MHz, d₆-DMSO) δ 11.55 (br s, 1H), 10.48 (s, 1H), 7.87-7.96 (m, 2H), 7.62 (ddd, J=8.2, 2.0, 1.0 Hz, 1H), 7.36-7.48 (m, 2H), 7.19 (ddd, J=8.0, 2.0, 0.9 Hz, 1H), 6.93-7.03 (m, 2H); MS (ESI) C₁₃H₁₁ClNO₂ [MH]⁺ m/z expected=248.05, observed=248.11; HPLC-1=>99%; HPLC-2=>99%.

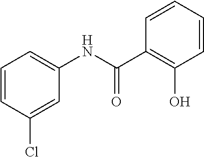 [SMILES: O=C(Nc1cccc(Cl)c1)c1ccccc1O]

### Example 14

Analog 13: Preparation of 3,5-Dibromo-2-hydroxy-N-phenylbenzamide. To a stirring mixture 41 (149 mg, 0.386 mmol) in anhydrous DCM (5 mL) was added BBr₃ (1.15 mL of 1 M in DCM, 1.15 mmol). The reaction was allowed to stir at R.T. (under Ar) for 3 days and then quenched with MeOH. The product was extracted into EtOAc and the organics were rinsed with brine, dried over Na₂SO₄, filtered, and concentrated. Flash chromatographic purification (hexanes:EtOAc gradient) afforded 13 as a tan solid (128 mg, 66% yield). ¹H-NMR (300 MHz, d₆-DMSO) δ 12.90-13.10 (br s, 1H), 10.65 (s, 1H), 8.31 (d, J=2.3 Hz, 1H), 8.03 (d, J=2.2 Hz, 1H), 7.64-7.71 (m, 2H), 7.38-7.45 (m, 2H), 7.17-7.24 (m, 1H); MS (ESI) C₁₃H₈Br₂NO₂ [M-H]⁻ m/z expected=367.9, observed=367.7; HPLC-1=98%; HPLC-2=>99%.

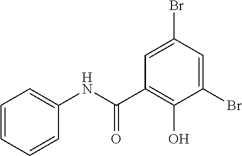 [SMILES: O=C(Nc1ccccc1)c1cc(Br)cc(Br)c1O]

### Example 15

Analog 14: Preparation of 3-Bromo-2-hydroxy-N-phenylbenzamide. To a stirring mixture 42 (186 mg, 0.608 mmol) in anhydrous DCM (5 mL) was added BBr₃ (1.80 mL of 1 M in DCM, 1.80 mmol). The reaction was allowed to stir at R.T. (under Ar) for 18 h and then quenched with MeOH. Reverse-phase flash chromatographic purification (H₂O:MeOH gradient) afforded 14 as a white solid (162 mg, 91% yield). ¹H-NMR (300 MHz, d₆-DMSO) δ 13.01 (s, 1H), 10.59 (s, 1H), 8.08 (dd, J=8.1, 1.4 Hz, 1H), 7.81 (dd, J=7.9, 1.4 Hz, 1H), 7.65-7.73 (m, 2H), 7.35-7.45 (m, 2H), 7.14-7.24 (m, 1H), 6.95 (t, J=7.9 Hz, 1H); MS (ESI) C₁₃H₁₁BrNO₂ [MH]⁺ m/z expected=292.0, observed=292.1; HPLC-1=>99%; HPLC-2=>99%.

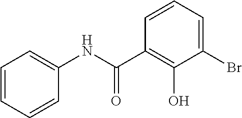 [SMILES: O=C(Nc1ccccc1)c1cccc(Br)c1O]

### Example 16

Analog 15: Preparation of 5-Bromo-2-hydroxy-N-phenylbenzamide. To a stirring mixture 43 (138 mg, 0.451 mmol) in anhydrous DCM (5 mL) was added BBr₃ (1.35 mL of 1 M in DCM, 1.35 mmol). The reaction was allowed to stir at R.T. (under Ar) for 18 h and then quenched with MeOH. Reverse-phase flash chromatographic purification (H₂O:MeOH gradient) afforded 15 as a white solid (63.3 mg, 48% yield). ¹H-NMR (300 MHz, d₆-DMSO) δ 11.89 (br s, 1H), 10.41 (s, 1H), 8.07 (d, J=2.5 Hz, 1H), 7.66-7.74 (m, 2H), 7.58 (dd, J=8.8, 2.5 Hz, 1H), 7.38 (t, J=7.9 Hz, 2H), 7.09-7.19 (m, 1H), 6.96 (d, J=8.8 Hz, 1H); MS (ESI) C₁₃H₁₁BrNO₂ [MH]⁺ m/z expected=292.0, observed=292.1; HPLC-1=>99%; HPLC-2=>99%.

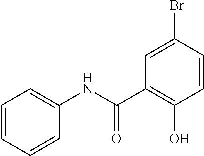 [SMILES: O=C(Nc1ccccc1)c1cc(Br)ccc1O]

### Example 17

Analog 16: Preparation of 2-Hydroxy-N-phenylbenzamide. To a stirring mixture 44 (153 mg, 0.673 mmol) in anhydrous DCM (5 mL) was added BBr₃ (2.00 mL of 1 M in DCM, 2.00 mmol). The reaction was allowed to stir at R.T. (under Ar) for 18 h and then quenched with MeOH. Reverse-phase flash chromatographic purification (H₂O:MeOH gradient) afforded 16 as an off-white solid (95.5 mg, 67% yield). ¹H-NMR (300 MHz, d₆-DMSO) δ 11.81 (br s, 1H), 10.39 (s, 1H), 7.96 (dd, J=7.8, 1.6 Hz, 1H), 7.67-7.74 (m, 2H), 7.34-7.46 (m, 3H), 7.10-7.17 (m, 1H), 6.93-7.01 (m, 2H); MS (ESI) C₁₃H₁₂NO₂ [MH]⁺ m/z expected=214.1, observed=214.2; HPLC-1=>99%; HPLC-2=>99%.

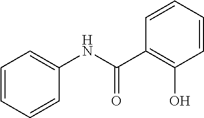 [SMILES: O=C(Nc1ccccc1)c1ccccc1O]

### Example 18

Analog 17: Preparation of N-(4-(Benzo[d]thiazol-2-ylthio)-3-chlorophenyl)-3,5-dibromobenzamide. 3,5-Dibromobenzoic acid (175 mg, 0.624 mmol) was stirred in anhydrous DCM (5 mL) with DCC (128 mg, 0.619 mmol) and DMAP (6.5 mg, 0.053 mmol) at R.T. for 1 h (under Ar). Compound 46 (150 mg, 0.514 mmol) was then added and the reaction was stirred for an additional 18 h. Flash chromatgraphic purification (hexanes:EtOAc gradient), followed by preparatory RP-HPLC purification, afforded 17 as a white solid (101 mg, 35% yield). ¹H-NMR (300 MHz, d₆-DMSO) δ 10.83 (br s, 1H), 8.24 (d, J=2.1 Hz, 1H), 8.13-8.18 (m, 3H), 7.83-8.01 (m, 4H), 7.47 (td, J=7.7, 1.3 Hz, 1H), 7.32-7.38 (m, 1H); MS (ESI) C₂₀H₁₀Br₂ClN₂OS₂ [M−H]⁻ m/z expected=550.8, observed=550.6; HPLC-1=98%; HPLC-2=98%.

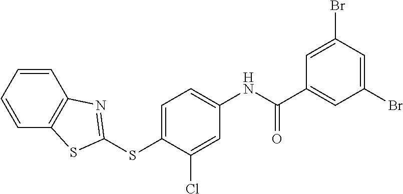 [SMILES: O=C(Nc1ccc(Sc2nc3ccccc3s2)c(Cl)c1)c1cc(Br)cc(Br)c1]

### Example 19

Analog 18: Preparation of N-(4-(Benzo[d]thiazol-2-ylthio)-3-chlorophenyl)-3-bromobenzamide. 3-Bromobenzoyl chloride (81.0 μL, 0.613 mmol), pyridine (61.0 μL, 0.748 mmol), and compound 46 (151 mg, 0.515 mmol) were stirred in anhydrous DCM (5 mL) at R.T. for 18 h (under Ar). Flash chromatographic purification (hexanes:EtOAc gradient) afforded 18 as a pale-yellow solid (226 mg, 92% yield). ¹H-NMR (300 MHz, d₆-DMSO) δ 10.79 (s, 1H), 8.28 (d, J=2.0 Hz, 1H), 8.18 (t, J=1.8 Hz, 1H), 7.90-8.00 (m, 4H), 7.83-7.88 (m, 2H), 7.54 (t, J=7.9 Hz, 1H), 7.43-7.59 (m, 1H), 7.32-7.38 (m, 1H); MS (ESI) C₂₀H₁₁BrClN₂OS₂ [M-H]⁻ m/z expected=472.9, observed=472.7; HPLC-1=98%; HPLC-2=98%.

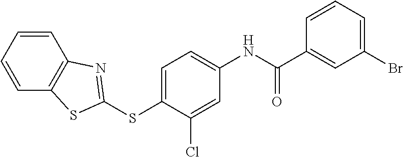 [SMILES: O=C(Nc1ccc(Sc2nc3ccccc3s2)c(Cl)c1)c1cccc(Br)c1]

### Example 20

Analog 19: Preparation of N-(4-(Benzo[d]thiazol-2-ylthio)-3-chlorophenyl)benzamide. Benzoyl chloride (71.0 μL, 0.616 mmol), pyridine (61.0 μL, 0.748 mmol), and compound 46 (153 mg, 0.522 mmol) were stirred in anhydrous DCM (5 mL) at R.T. for 18 h (under Ar). Flash chromatographic purification (hexanes:EtOAc gradient) afforded 19 as a pale-yellow solid (188 mg, 91% yield). ¹H-NMR (300 MHz, d₆-DMSO) δ 10.72 (s, 1H), 8.31 (d, J=1.6 Hz, 1H), 7.92-8.03 (m, 5H), 7.86 (d, J=7.6 Hz, 1H), 7.54-7.67 (m, 3H), 7.43-7.50 (m, 1H), 7.32-7.38 (m, 1H); MS (ESI) C₂₀H₁₂ClN₂OS₂ [M-H]⁻ m/z expected=395.0, observed=394.9; HPLC-1=99%; HPLC-2=99%.

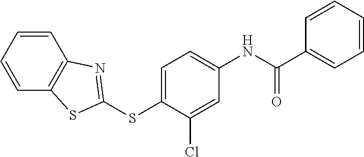 [SMILES: O=C(Nc1ccc(Sc2nc3ccccc3s2)c(Cl)c1)c1ccccc1]

### Example 21

Analog 20: Preparation of N-(4-(Benzo[d]thiazol-2-ylthio)phenyl)-3,5-dibromobenzamide. 3,5-Dibromobenzoic acid (120 mg, 0.428 mmol), compound 47 (85.8 mg, 0.332 mmol), EDC (88.9 mg, 0.464 mmol), HOBt.H₂O (82.5 mg, 0.539 mmol), and TEA (69.5 L, 0.499 mmol) were stirred in anhydrous DCM (5 mL) at R.T. for 18 h (under Ar). Flash chromatographic purification (hexanes:EtOAc gradient) afforded 20 as an off-white solid (27.2 mg, 16% yield). ¹H-NMR (300 MHz, d₆-DMSO) δ 10.73 (s, 1H), 8.17 (d, J=2.0 Hz, 2H), 8.13-8.15 (m, 1H), 7.91-8.02 (m, 3H), 7.80-7.87 (m, 3H), 7.42-7.48 (m, 1H), 7.30-7.37 (m, 1H); MS (ESI) C₂₀H₁₁Br₂N₂OS₂ [M-H]⁻ m/z expected=516.9, observed=516.7; HPLC-1=>99%; HPLC-2=>99%.

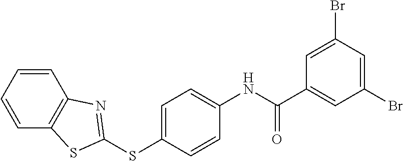 [SMILES: O=C(Nc1ccc(Sc2nc3ccccc3s2)cc1)c1cc(Br)cc(Br)c1]

### Example 22

Analog 21: Preparation of N-(4-(Benzo[d]thiazol-2-ylthio)phenyl)-3-bromobenzamide. 3-Bromobenzoyl chloride (65.0 μL, 0.492 mmol), pyridine (40.0 μL, 0.491 mmol), and compound 47 (116 mg, 0.449 mmol) were stirred in anhydrous DCM (5 mL) at R.T. for 18 h (under Ar). Flash chromatographic purification (hexanes:EtOAc gradient) afforded 21 as a white solid (191 mg, 96% yield). ¹H-NMR (300 MHz, d₆-DMSO) δ 10.68 (s, 1H), 8.17 (d, J=1.7 Hz, 1H), 7.96-8.03 (m, 3H), 7.91-7.95 (m, 1H), 7.79-7.86 (m, 4H), 7.50-7.57 (m, 1H), 7.45 (td, J=7.7, 1.3 Hz, 1H), 7.30-7.37 (m, 1H); MS (ESI) C₂₀H₁₂BrN₂OS₂ [M-H]⁻ m/z expected=439.0, observed=438.8; HPLC-1=99%; HPLC-2=>99%.

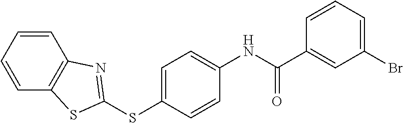 [SMILES: O=C(Nc1ccc(Sc2nc3ccccc3s2)cc1)c1cccc(Br)c1]

### Example 23

Analog 22: Preparation of N-(4-(Benzo[d]thiazol-2-ylthio)phenyl)benzamide. Benzoyl chloride (57.0 μL, 0.495 mmol), pyridine (44.0 μL, 0.540 mmol), and compound 47 (117 mg, 0.451 mmol) were stirred in anhydrous DCM (5 mL) at R.T. for 18 h (under Ar). Flash chromatographic purification (hexanes:EtOAc gradient) afforded 22 as a white solid (159 mg, 97% yield). ¹H-NMR (300 MHz, d₆-DMSO) δ 10.60 (s, 1H), 7.96-8.04 (m, 4H), 7.90-7.95 (m, 1H), 7.77-7.86 (m, 3H), 7.73-7.66 (m, 3H), 7.45 (td, J=7.7, 1.2 Hz, 1H), 7.30-7.37 (m, 1H); MS (ESI) C₂₀H₁₃N₂OS₂ [M-H]⁻ m/z expected=361.1, observed=361.0; HPLC-1=>99%; HPLC-2 =97%.

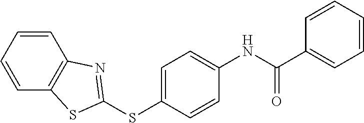 [SMILES: O=C(Nc1ccc(Sc2nc3ccccc3s2)cc1)c1ccccc1]

### Example 24

Analog 23: Preparation of 3,5-Dibromo-N-(3-chlorophenyl)benzamide. 3,5-Dibromobenzoic acid (299 mg, 1.07 mmol) was stirred in SOCl₂ (5 mL) at 60° C. for 1 h, then was concentrated. Anhydrous DCM (5 mL), 3-chloroaniline (94.0 μL, 0.892 mmol), and pyridine (87.0 μL, 1.07 mmol) were added and the reaction was stirred at R.T. for 18 h (under Ar). Flash chromatographic purification (hexanes:EtOAc gradient) afforded 23 as an off-white solid (150 mg, 43% yield). ¹H-NMR (300 MHz, d₆-DMSO) δ 10.55 (s, 1H), 8.13 (d, J=1.8 Hz, 2H), 8.02 (d, J=1.9 Hz, 1H), 7.92 (t, J=2.0 Hz, 1H), 7.65-7.71 (m, 1H), 7.40 (t, J=8.1 Hz, 1H), 7.19 (ddd, J=8.0, 2.1, 0.9 Hz, 1H); MS (ESI) C₁₃H₉Br₂ClNO [MH]⁺ m/z expected=389.9, observed=390.0; HPLC-1=97%; HPLC-2=97%.

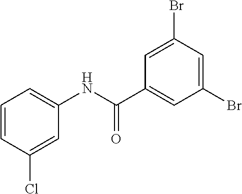 [SMILES: O=C(Nc1cccc(Cl)c1)c1cc(Br)cc(Br)c1]

### Example 25

Analog 24: Preparation of 3-Bromo-N-(3-chlorophenyl)benzamide. 3-Bromobenzoyl chloride (0.23 mL, 1.7 mmol), pyridine (0.14 mL, 1.7 mmol), and 3-chloroaniline (0.15 mL, 1.4 mmol) were stirred in anhydrous DCM (5 mL) at R.T. for 18 h (under Ar). Flash chromatographic purification (hexanes:EtOAc gradient) afforded 24 as an off-white solid (338 mg, 77% yield). ¹H-NMR (300 MHz, d₆-DMSO) δ 10.50 (s, 1H), 8.14 (t, J=1.8 Hz, 1H), 7.92-7.98 (m, 2H), 7.78-7.84 (m, 1H), 7.70 (ddd, J=8.2, 2.0, 0.9 Hz, 1H), 7.51 (t, J=8.1 Hz, 1H), 7.39 (t, J=8.1 Hz, 1H), 7.18 (ddd, J=8.0, 2.0, 0.9 Hz, 1H); MS (ESI) C₁₃H₁₀BrClNO [MH]⁺ m/z expected=312.0, observed=312.0; HPLC-1=>99%; HPLC-2=>99%.

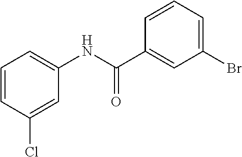 [SMILES: O=C(Nc1cccc(Cl)c1)c1cccc(Br)c1]

### Example 26

Analog 25: Preparation of N-(3-Chlorophenyl)benzamide. Benzoyl chloride (0.26 mL, 1.7 mmol), pyridine (0.19 mL, 2.3 mmol), and 3-chloroaniline (0.20 mL, 2.3 mmol) were stirred in anhydrous DCM (5 mL) at R.T. for 18 h (under Ar). Flash chromatographic purification (hexanes:EtOAc gradient) afforded 25 as an off-white solid (414 mg, 94% yield). ¹H-NMR (300 MHz, d₆-DMSO) δ 10.42 (s, 1H), 7.93-7.99 (m, 3H), 7.71 (ddd, J=8.2, 1.9, 0.9 Hz, 1H), 7.51-7.64 (m, 3H), 7.39 (t, J=8.1 Hz, 1H), 7.16 (ddd, J=8.0, 2.1, 0.9 Hz, 1H); MS (ESI) C₁₃H₁₁ClNO [MH]⁺ m/z expected=232.1, observed=232.0; HPLC-1=>99%; HPLC-2=>99%.

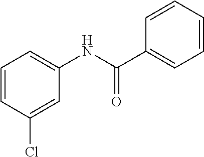 [SMILES: O=C(Nc1cccc(Cl)c1)c1ccccc1]

### Example 27

Analog 26: Preparation of 3,5-Dibromo-N-phenylbenzamide. 3,5-Dibromobenzoic acid (312 mg, 1.11 mmol) was stirred in SOCl₂ (5 mL) at 60° C. for 1 h, then was concentrated. Anhydrous DCM (5 mL), aniline (85.0 μL, 0.931 mmol), and pyridine (90.0 μL, 1.10 mmol) were added and the reaction was stirred at R.T. for 18 h (under Ar). Flash chromatographic purification (hexanes:EtOAc gradient) afforded 26 as a white solid (224 mg, 68% yield). ¹H-NMR (300 MHz, d₆-DMSO) δ 10.42 (s, 1H), 8.14 (d, J=1.7 Hz, 2H), 8.07-8.11 (m, 1H), 7.72-7.78 (m, 2H), 7.33-7.41 (m, 2H), 7.09-7.16 (m, 1H); MS (ESI) C₁₃H₁₀Br₂NO [MH]⁺ m/z expected=355.9, observed=356.0; HPLC-1=>99%; HPLC-2=>99%.

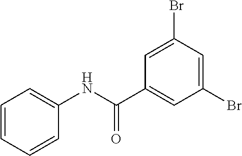 [SMILES: O=C(Nc1ccccc1)c1cc(Br)cc(Br)c1]

### Example 28

Analog 27: Preparation of 3-Bromo-N-phenylbenzamide. 3-Bromobenzoyl chloride (0.17 mL, 1.3 mmol), pyridine (0.11 mL, 1.3 mmol), and aniline (0.10 mL, 1.1 mmol) were stirred in anhydrous DCM (5 mL) at R.T. for 18 h (under Ar). Flash chromatographic purification (hexanes:EtOAc gradient) afforded 27 as a white solid (289 mg, 95% yield). ¹H-NMR (300 MHz, d₆-DMSO) δ 10.35 (s, 1H), 8.14 (t, J=1.8 Hz, 1H), 7.95 (dt, J=7.8, 1.3 Hz, 1H), 7.73-7.83 (m, 3H), 7.50 (t, J=7.9 Hz, 1H), 7.32-7.39 (m, 2H), 7.08-7.16 (m, 1H); MS (ESI) C₁₃H₁₁BrNO [MH]⁺ m/z expected=276.0, observed=276.1; HPLC-1=98%; HPLC-2=98%.

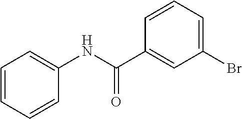 [SMILES: O=C(Nc1ccccc1)c1cccc(Br)c1]

### Example 29

Analog 28: Preparation of N-Phenylbenzamide. Benzoyl chloride (0.30 mL, 2.6 mmol), pyridine (0.22 mL, 2.7 mmol), and aniline (0.20 mL, 2.2 mmol) were stirred in anhydrous DCM (5 mL) at R.T. for 18 h (under Ar). Flash chromatographic purification (hexanes:EtOAc gradient) afforded 28 as a white solid (367 mg, 85% yield). ¹H-NMR (300 MHz, d₆-DMSO) δ 10.26 (s, 1H), 7.93-7.98 (m, 2H), 7.75-7.81 (m, 2H), 7.49-7.63 (m, 3H), 7.32-7.39 (m, 2H), 7.06-7.13 (m, 1H); MS (ESI) C₁₃H₁₂NO [MH]⁺ m/z expected=198.1, observed=198.0; HPLC-1=>99%; HPLC-2=>99%.

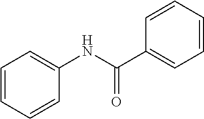 [SMILES: O=C(Nc1ccccc1)c1ccccc1]

### Example 30

Analog 29: Preparation of N-(4-(Benzo[d]thiazol-2-ylthio)-3-chlorophenyl)-3,5-dibromo-2-methoxybenzamide. Compound 48 (225 mg, 0.726 mmol) was stirred in SOCl₂ (2 mL) at 60° C. for 1 h, then was concentrated. Anhydrous DCM (5 mL), compound 46 (148 mg, 0.505 mmol), and pyridine (62.0 μL, 0.760 mol) were added and the reaction was stirred at R.T. for 18 h (under Ar). Flash chromatographic purification (hexanes:EtOAc gradient) afforded 29 as a yellow solid (58.6 mg, 20% yield). ¹H-NMR (300 MHz, d₆-DMSO) δ 11.00 (s, 1H), 8.21 (d, J=2.1 Hz, 1H), 8.10 (d, J=2.3 Hz, 1H), 7.97 (t, J=8.3 Hz, 2H), 7.82-7.87 (m, 2H), 7.78 (dd, J=8.6, 2.2 Hz, 1H), 7.47 (td, J=7.7, 1.3 Hz, 1H), 7.32-7.38 (m, 1H), 3.84 (s, 3H); MS (ESI) C₂₁H₁₂Br₂ClN₂O₂S₂ [M-H]⁻ m/z expected=580.8, observed=580.7; HPLC-1=>99%; HPLC-2=>99%.

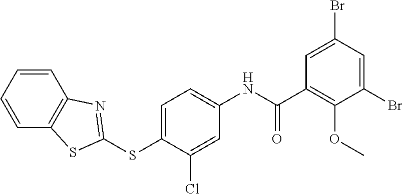 [SMILES: COc1c(Br)cc(Br)cc1C(=O)Nc1ccc(Sc2nc3ccccc3s2)c(Cl)c1]

### Example 31

Analog 30: Preparation of N-(4-(Benzo[d]thiazol-2-ylthio)-3-chlorophenyl)-3-bromo-2-methoxybenzamide. 3-Bromo-2-methoxybenzoic acid (171 mg, 0.741 mmol) was stirred in SOCl₂ (2 mL) at 60° C. for 1 h, then was concentrated. Anhydrous DCM (5 mL), compound 46 (170 mg, 0.581 mmol), and pyridine (60.5 μL, 0.742 mol) were added and the reaction was stirred at R.T. for 18 h (under Ar). Flash chromatographic purification (hexanes:EtOAc gradient) afforded 30 as a yellow solid (48.6 mg, 17% yield). ¹H-NMR (300 MHz, d₆-DMSO) δ 10.94 (s, 1H), 8.24 (d, J=2.1 Hz, 1H), 7.93-8.00 (m, 2H), 7.79-7.87 (m, 3H), 7.61 (dd, J=7.6, 1.6 Hz, 1H), 7.47 (td, J=7.7, 1.3 Hz, 1H), 7.32-7.39 (m, 1H), 7.24 (t, J=7.8 Hz, 1H), 3.84 (s, 3H); MS (ESI) C₂₁H₁₃BrClN₂O₂S₂ [M-H]⁻ m/z expected=502.9, observed=502.7; HPLC-1=98%; HPLC-2=98%.

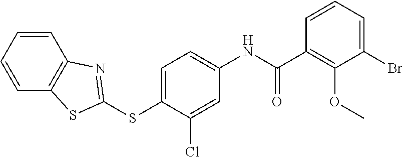 [SMILES: COc1c(Br)cccc1C(=O)Nc1ccc(Sc2nc3ccccc3s2)c(Cl)c1]

### Example 32

Analog 31: Preparation of N-(4-(Benzo[d]thiazol-2-ylthio)-3-chlorophenyl)-5-bromo-2-methoxybenzamide. Compound 49 (1.13 g, 4.88 mmol) was stirred in SOCl₂ (3 mL) at 60° C. for 1 h, then was concentrated. Anhydrous DCM (10 mL), compound 46 (1.18 g, 4.04 mmol), and pyridine (0.49 mL, 6.0 mmol) were added and the reaction was stirred at R.T. for 18 h (under Ar). The reaction was then diluted into hexanes and the precipitate was filtered, rinsed with 1 M HCl and water, and dried. Flash chromatographic purification (hexanes:EtOAc gradient) afforded 31 as a yellow solid (1.80 g, 88% yield). ¹H-NMR (300 MHz, d₆-DMSO) δ 10.70 (s, 1H), 8.22 (d, J=2.0 Hz, 1H), 7.93-7.99 (m, 2H), 7.80-7.87 (m, 2H), 7.67-7.76 (m, 2H), 7.47 (td, J=7.7, 1.3 Hz, 1H), 7.32-7.38 (m, 1H), 7.19 (d, J=8.8 Hz, 1H), 3.89 (s, 3H); MS (ESI) C₂₁H₁₃BrClN₂O₂S₂ [M-H]⁻ m/z expected=502.9, observed=502.7; HPLC-1=98%; HPLC-2=98%.

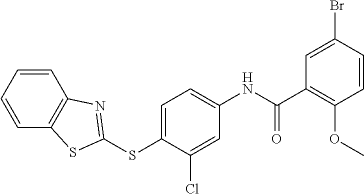 [SMILES: COc1ccc(Br)cc1C(=O)Nc1ccc(Sc2nc3ccccc3s2)c(Cl)c1]

### Example 33

Analog 32: Preparation of N-(4-(Benzo[d]thiazol-2-ylthio)-3-chlorophenyl)-2-methoxybenzamide. 2-Methoxybenzoic acid (151 mg, 0.990 mmol) was stirred in SOCl₂ (1 mL) at 60° C. for 1 h, then was concentrated. Anhydrous DCM (5 mL), compound 46 (192 mg, 0.654 mmol), and pyridine (80.0 μL, 0.981 mmol) were added and the reaction was stirred at R.T. for 18 h (under Ar). Flash chromatographic purification (hexanes:EtOAc gradient), followed by preparatory RP-HPLC purification, afforded 32 as an off-white solid (87.3 mg, 31% yield). ¹H-NMR (300 MHz, d₆-DMSO) δ 10.62 (s, 1H), 8.26 (d, J=2.0 Hz, 1H), 7.91-7.98 (m, 2H), 7.82-7.90 (m, 2H), 7.63 (dd, J=7.5, 1.7 Hz, 1H), 7.51-7.57 (m, 1H), 7.43-7.49 (m, 1H), 7.31-7.38 (m, 1H), 7.21 (d, J=8.3 Hz, 1H), 7.05-7.14 (m, 1H), 3.89 (s, 3H); MS (ESI) C₂₁H₁₄ClN₂O₂S₂ [MH]⁺ m/z expected=427.0, observed=427.0; HPLC-1=99%; HPLC-2=97%.

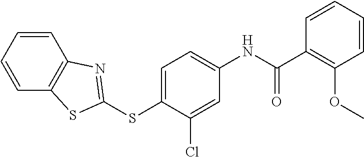 [SMILES: COc1ccccc1C(=O)Nc1ccc(Sc2nc3ccccc3s2)c(Cl)c1]

### Example 34

Analog 33: Preparation of N-(4-(Benzo[d]thiazol-2-ylthio)phenyl)-3,5-dibromo-2-methoxybenzamide. Compound 48 (172 mg, 0.555 mmol) was stirred in anhydrous DCM (5 mL) with DCC (120 mg, 0.579 mmol) and DMAP (9.0 mg, 0.074 mmol) at R.T. for 1 h (under Ar). Compound 47 (121 mg, 0.469 mmol) was then added and the reaction was stirred for an additional 18 h. Flash chromatgraphic purification (hexanes:EtOAc gradient) afforded 33 as a white solid (221 mg, 86% yield). ¹H-NMR (300 MHz, d₆-DMSO) δ 10.85 (s, 1H), 8.08 (d, J=2.3 Hz, 1H), 7.89-7.96 (m, 3H), 7.79-7.86 (m, 4H), 7.45 (td, J=7.7, 1.3 Hz, 1H), 7.30-7.37 (m, 1H), 3.84 (s, 3H); MS (ESI) C₂₁H₁₃Br₂N₂O₂S₂ [M-H]⁻ m/z expected=546.9, observed=456.7; HPLC-1=97%; HPLC-2=97%.

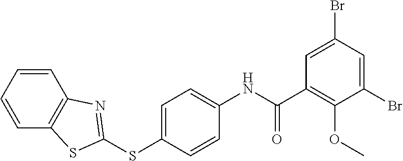 [SMILES: COc1c(Br)cc(Br)cc1C(=O)Nc1ccc(Sc2nc3ccccc3s2)cc1]

### Example 35

Analog 34: Preparation of N-(4-(Benzo[d]thiazol-2-ylthio)phenyl)-3-bromo-2-methoxybenzamide. 3-Bromo-2-methoxybenzoic acid (160 mg, 0.692 mmol) was stirred in anhydrous DCM (5 mL) with DCC (150 mg, 0.727 mmol) and DMAP (10.0 mg, 0.0819 mmol) at R.T. for 1 h (under Ar). Compound 47 (150 mg, 0.580 mmol) was then added and the reaction was stirred for an additional 18 h. Flash chromatgraphic purification (hexanes:EtOAc gradient) afforded 34 as a white solid (184 mg, 67% yield). ¹H-NMR (300 MHz, d₆-DMSO) δ 10.78 (s, 1H), 7.91-7.97 (m, 3H), 7.78-7.86 (m, 4H), 7.59 (dd, J=7.6, 1.5 Hz, 1H), 7.45 (td, J=7.7, 1.2 Hz, 1H), 7.30-7.37 (m, 1H), 7.19-7.26 (m, 1H), 3.84 (s, 3H); MS (ESI) C₂₁H₁₄BrN₂O₂S₂ [M-H]⁻ m/z expected=469.0, observed=468.8; HPLC-1=>99%; HPLC-2=>99%.

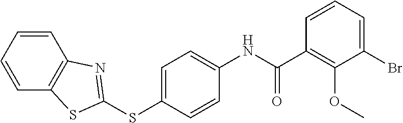 [SMILES: COc1c(Br)cccc1C(=O)Nc1ccc(Sc2nc3ccccc3s2)cc1]

### Example 36

Analog 35: Preparation of N-(4-(Benzo[d]thiazol-2-ylthio)phenyl)-5-bromo-2-methoxybenzamide. Compound 49 (154 mg, 0.667 mmol) was stirred in SOCl₂ (1 mL) at 60° C. for 1 h, then was concentrated. Anhydrous DCM (5 mL), compound 47 (117 g, 0.453 mmol), and pyridine (55.0 μL, 0.674 mmol) were added and the reaction was stirred at R.T. for 18 h (under Ar). Flash chromatographic purification (hexanes:EtOAc gradient) afforded 35 as an off-white solid (199 mg, 93% yield). ¹H-NMR (300 MHz, d₆-DMSO) δ 10.56 (s, 1H), 7.90-7.97 (m, 3H), 7.77-7.86 (m, 3H), 7.66-7.74 (m, 2H), 7.42-7.48 (m, 1H), 7.30-7.37 (m, 1H), 7.18 (d, J=8.8 Hz, 1H), 3.89 (s, 3H); MS (ESI) C₂₁H₁₄BrN₂O₂S₂ [M-H]⁻ m/z expected=469.0, observed=468.8; HPLC-1=>99%; HPLC-2=>99%.

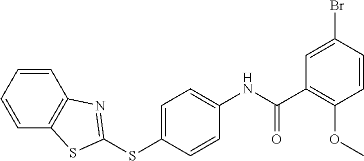 [SMILES: COc1ccc(Br)cc1C(=O)Nc1ccc(Sc2nc3ccccc3s2)cc1]

### Example 37

Analog 36: Preparation of N-(4-(Benzo[d]thiazol-2-ylthio)phenyl)-2-methoxybenzamide. 2-Methoxybenzoic acid (150 mg, 0.988 mmol) was stirred in SOCl₂ (1 mL) at 60° C. for 1 h, then was concentrated. Anhydrous DCM (5 mL), compound 47 (170 mg, 0.657 mmol), and pyridine (81.0 μL, 0.993 mmol) were added and the reaction was stirred at R.T. for 18 h (under Ar). Flash chromatographic purification (hexanes:EtOAc gradient), followed by preparatory RP-HPLC purification, afforded 36 as a white solid (81.3 mg, 32% yield). ¹H-NMR (300 MHz, d₆-DMSO) δ 10.48 (s, 1H), 7.90-7.99 (m, 3H), 7.76-7.86 (m, 3H), 7.62 (dd, J=7.5, 1.6 Hz, 1H), 7.49-7.57 (m, 1H), 7.42-7.49 (m, 1H), 7.29-7.37 (m, 1H), 7.20 (d, J=8.3 Hz, 1H), 7.08 (t, J=7.4 Hz, 1H), 3.91 (s, 3H); MS (ESI) C₂₁H₁₅N₂O₂S₂[M-H]⁻ m/z expected=391.1, observed=390.9; HPLC-1=>99%; HPLC-2=>99%.

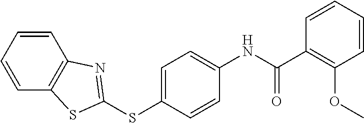 [SMILES: COc1ccccc1C(=O)Nc1ccc(Sc2nc3ccccc3s2)cc1]

### Example 38

Analog 37: Preparation of 3,5-Dibromo-N-(3-chlorophenyl)-2-methoxybenzamide. Compound 48 (259 mg, 0.835 mmol) was stirred in SOCl₂ (2 mL) at 60° C. for 1 h, then was concentrated. Anhydrous DCM (5 mL), 3-chloroaniline (73.0 μL, 0.694 mmol), and pyridine (74.0 μL, 0.907 mol) were added and the reaction was stirred at R.T. for 18 h (under Ar). Flash chromatographic purification (hexanes:EtOAc gradient) afforded 37 as an off-white solid (280 mg, 80% yield). ¹H-NMR (300 MHz, d₆-DMSO) δ 10.68 (s, 1H), 8.06 (d, J=2.4 Hz, 1H), 7.90 (t, J=2.0 Hz, 1H), 7.77 (d, J=2.4 Hz, 1H), 7.57 (d, J=8.2 Hz, 1H), 7.39 (t, J=8.1 Hz, 1H), 7.16-7.22 (m, 1H), 3.81 (s, 3H); MS (ESI) C₁₄H₉Br₂ClNO₂ [M-H]⁻ m/z expected=415.9, observed=415.7; HPLC-1=99%; HPLC-2=97%.

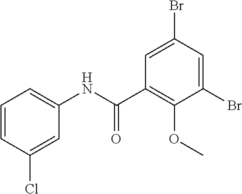 [SMILES: COc1c(Br)cc(Br)cc1C(=O)Nc1cccc(Cl)c1]

### Example 39

Analog 38: Preparation of 3,5-Dibromo-N-(3-chlorophenyl)-2-methoxybenzamide. 3-Bromo-2-methoxybenzoic acid (382 mg, 1.65 mmol) was stirred in SOCl₂ (3 mL) at 60° C. for 1 h, then was concentrated. Anhydrous DCM (5 mL), 3-chloroaniline (0.15 mL, 1.4 mmol), and pyridine (0.13 mL, 1.6 mmol) were added and the reaction was stirred at R.T. for 18 h (under Ar). Flash chromatographic purification (hexanes:EtOAc gradient) afforded 38 as a white solid (303 mg, 63% yield). ¹H-NMR (300 MHz, d₆-DMSO) δ 10.60 (s, 1H), 7.91-7.95 (m, 1H), 7.80 (dd, J=8.0, 1.6 Hz, 1H), 7.57-7.62 (m, 1H), 7.55 (dd, J=7.6, 1.6 Hz, 1H), 7.38 (t, J=8.1 Hz, 1H), 7.15-7.23 (m, 2H), 3.81 (s, 3H); MS (ESI) C₁₄H₁₂BrClNO₂ [MH]⁺ m/z expected=342.0, observed=342.1; HPLC-1=>99%; HPLC-2=>99%.

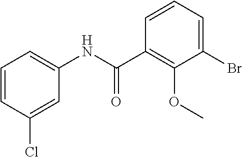 [SMILES: COc1c(Br)cccc1C(=O)Nc1cccc(Cl)c1]

### Example 40

Analog 39: Preparation of 5-Bromo-N-(3-chlorophenyl)-2-methoxybenzamide. Compound 49 (327 mg, 1.42 mmol) was stirred in SOCl₂ (3 mL) at 60° C. for 1 h, then was concentrated. Anhydrous DCM (5 mL), 3-chloroaniline (0.12 mL, 1.1 mmol), and pyridine (0.12 mL, 1.5 mmol) were added and the reaction was stirred at R.T. for 18 h (under Ar). Flash chromatographic purification (hexanes:EtOAc gradient) afforded 39 as an off-white solid (345 mg, 89% yield). ¹H-NMR (300 MHz, d₆-DMSO) δ 10.37 (s, 1H), 7.91 (t, J=2.0 Hz, 1H), 7.65-7.72 (m, 2H), 7.57-7.63 (m, 1H), 7.37 (t, J=8.1 Hz, 1H), 7.13-7.19 (m, 2H), 3.87 (s, 3H(; MS (ESI) C₁₄H₁₂BrClNO₂ [MH]⁺ m/z expected=342.0, observed=342.1; HPLC-1=>99%; HPLC-2=>99%.

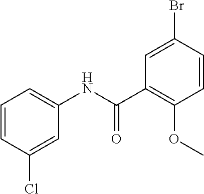 [SMILES: COc1ccc(Br)cc1C(=O)Nc1cccc(Cl)c1]

### Example 41

Analog 40: Preparation of N-(3-Chlorophenyl)-2-methoxybenzamide. 2-Methoxybenzoic acid (189 mg, 1.24 mmol) was stirred in SOCl₂ (2 mL) at 60° C. for 1 h, then was concentrated. Anhydrous DCM (5 mL), 3-chloroaniline (0.11 mL, 1.0 mmol), and pyridine (0.10 mL, 1.2 mmol) were added and the reaction was stirred at R.T. for 18 h (under Ar). Flash chromatographic purification (hexanes:EtOAc gradient), followed by preparatory RP-HPLC purification, afforded 40 as a white solid (81.3 mg, 32% yield). ¹H-NMR (300 MHz, d₆-DMSO) δ 10.30 (s, 1H), 7.95 (t, J=2.0 Hz, 1H), 7.58-7.66 (m, 2H), 7.47-7.55 (m, 1H), 7.36 (t, J=8.1 Hz, 1H), 7.12-7.21 (m, 2H), 7.07 (td, J=7.5, 0.9 Hz, 1H), 3.89 (s, 3H); MS (ESI) C₁₄H₁₃ClNO₂ [MH]⁺ m/z expected=262.1, observed=262.1; HPLC-1=>99%; HPLC-2=>99%.

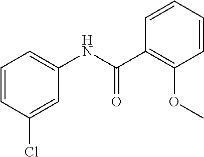 [SMILES: COc1ccccc1C(=O)Nc1cccc(Cl)c1]

### Example 42

Analog 41: Preparation of 3,5-Dibromo-2-methoxy-N-phenylbenzamide. Compound 48 (274 mg, 0.885 mmol) was stirred in SOCl₂ (2 mL) at 60° C. for 1 h, then was concentrated. Anhydrous DCM (5 mL), aniline (69.0 μL, 0.756 mmol), and pyridine (58.0 μL, 0.0.711 mol) were added and the reaction was stirred at R.T. for 18 h (under Ar). Flash chromatographic purification (hexanes:EtOAc gradient) afforded 41 as an off-white solid (258 mg, 80% yield). ¹H-NMR (300 MHz, d₆-DMSO) δ 10.49 (s, 1H), 8.04 (d, J=2.3 Hz, 1H), 7.75 (d, J=2.4 Hz, 1H), 7.67-7.73 (m, 2H), 7.36 (t, J=7.9 Hz, 2H), 7.08-7.16 (m, 1H), 3.81 (s, 3H); MS (ESI) C₁₄H₁₀Br₂NO₂ [M-H]⁻ m/z expected=381.9, observed=381.7; HPLC-1=>99%; HPLC-2=>99%.

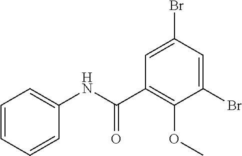 [SMILES: COc1c(Br)cc(Br)cc1C(=O)Nc1ccccc1]

### Example 43

Analog 42: Preparation of 3-Bromo-2-methoxy-N-phenylbenzamide. 3-Bromo-2-methoxybenzoic acid (356 mg, 1.54 mmol) was stirred in SOCl₂ (3 mL) at 60° C. for 1 h, then was concentrated. Anhydrous DCM (5 mL), aniline (0.12 mL, 1.3 mmol), and pyridine (0.13 mL, 1.6 mmol) were added and the reaction was stirred at R.T. for 18 h (under Ar). Flash chromatographic purification (hexanes:EtOAc gradient) afforded 42 as a white solid (315 mg, 79% yield). ¹H-NMR (300 MHz, d₆-DMSO) δ 10.40 (s, 1H), 7.78 (dd, J=8.0, 1.6 Hz, 1H), 7.69-7.75 (m, 2H), 7.54 (dd, J=7.6, 1.6 Hz, 1H), 7.32-7.38 (m, 2H), 7.19 (t, J=7.8 Hz, 1H), 7.07-7.14 (m, 1H), 3.82 (s, 3H); MS (ESI) C₁₄H₁₃BrNO₂ [MH]⁺ m/z expected=306.0, observed=306.1; HPLC-1=>99%; HPLC-2=>99%.

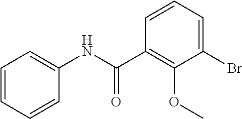 [SMILES: COc1c(Br)cccc1C(=O)Nc1ccccc1]

### Example 44

Analog 43: Preparation of 5-Bromo-2-methoxy-N-phenylbenzamide. Compound 49 (302 mg, 1.31 mmol) was stirred in SOCl₂ (3 mL) at 60° C. for 1 h, then was concentrated. Anhydrous DCM (5 mL), aniline (0.10 mL, 1.1 mmol), and pyridine (0.11 mL, 1.3 mmol) were added and the reaction was stirred at R.T. for 18 h (under Ar). Flash chromatographic purification (hexanes:EtOAc gradient) afforded 43 as a white solid (250 mg, 74% yield). ¹H-NMR (300 MHz, d₆-DMSO) δ 10.20 (s, 1H), 7.63-7.73 (m, 4H), 7.31-7.37 (m, 2H), 7.15 (d, J=8.8 Hz, 1H), 7.06-7.13 (m, 1H), 3.87 (s, 3H); MS (ESI) C₁₄H₁₃BrNO₂ [MH]⁺ m/z expected=306.0, observed=306.1; HPLC-1=>99%; HPLC-2=>99%.

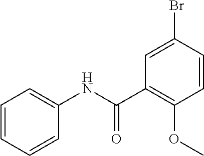 [SMILES: COc1ccc(Br)cc1C(=O)Nc1ccccc1]

### Example 45

Analog 44: Preparation of 2-Methoxy-N-phenylbenzamide. 2-Methoxybenzoic acid (178 mg, 1.17 mmol) was stirred in SOCl₂ (2 mL) at 60° C. for 1 h, then was concentrated. Anhydrous DCM (5 mL), aniline (0.10 mL, 1.1 mmol), and pyridine (0.10 mL, 1.2 mmol) were added and the reaction was stirred at R.T. for 18 h (under Ar). Flash chromatographic purification (hexanes:EtOAc gradient) afforded 44 as a white solid (235 mg, 94% yield). ¹H-NMR (300 MHz, d₆-DMSO) δ 10.12 (s, 1H), 7.71-7.77 (m, 2H), 7.62 (dd, J=7.5, 1.8 Hz, 1H), 7.46-7.54 (m, 1H), 7.30-7.37 (m, 2H), 7.18 (d, J=8.1 Hz, 1H), 7.04-7.12 (m, 2H), 3.89 (s, 3H); MS (ESI) C₁₄H₁₄NO₂ [MH]⁺ m/z expected=228.1, observed=228.1; HPLC-1=>99%; HPLC-2=>99%.

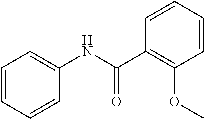 [SMILES: COc1ccccc1C(=O)Nc1ccccc1]

### Example 46

Analog 45: Preparation of 2-((2-Chloro-4-nitrophenyl)thio)benzo[d]thiazole. 2-Mercaptobenzothiazole (11.9 g, 71.1 mmol), 3,4-dichloronitrobenzene (11.8 g, 61.5 mmol), and potassium carbonate (11.7 g, 84.7 mmol) were stirred together in DMF (60 mL) at R.T. overnight, then at 80° C. for 4 h. The reaction was then diluted with water and the precipitate was filtered, rinsed with water, and dried to afford 45 as a yellow powder (19.5 g, 98% yield). ¹H-NMR (300 MHz, d₆-DMSO) δ 8.51 (d, J=2.4 Hz, 1H), 8.23 (dd, J=8.7, 2.5 Hz, 1H), 8.10 (d, J=7.8 Hz, 1H), 8.07-8.13 (m, 1H), 7.91 (d, J=8.7 Hz, 1H), 7.44-7.59 (m, 2H); MS (ESI) C₁₃H₈ClN₂O₂S₂ [MH]⁺ m/z expected=323.0, observed=323.1; HPLC-1=98%.

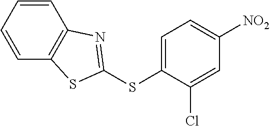 [SMILES: O=[N+]([O-])c1ccc(Sc2nc3ccccc3s2)c(Cl)c1]

### Example 47

Analog 46: Preparation of 46: 4-(Benzo[d]thiazol-2-ylthio)-3-chloroaniline. Tin powder (5.64 g, 47.5 mmol) was added slowly to a stirring mixture of 45 in a 1:10 mixture of HCl:AcOH (15 mL). The reaction was allowed to stir at R.T. for 2 days, then diluted with EtOAc and H₂O, neutralized with NaHCO₃, and filtered. The filtrate was extracted with EtOAc and the organics dried over Na₂SO₄, filtered, and concentrated. The crude product was then chromatographed over silica (hexanes:EtOAc gradient) and concentrated. The residue was diluted in a 4:1 mixture of hexanes:DCM and the precipitate was filtered and dried to afford 46 as a yellow powder (3.73 g, 81% yield). ¹H-NMR (300 MHz, d₆-DMSO) δ 7.87-7.96 (m, 1H), 7.80 (d, J=8.1 Hz, 1H), 7.53 (d, J=8.5 Hz, 1H), 7.43 (td, J=7.7, 1.3 Hz, 1H), 7.26-7.34 (m, 1H), 6.87 (d, J=2.4 Hz, 1H), 6.64 (dd, J=8.5, 2.4 Hz, 1H), 6.18 (s, 2H); MS (ESI) C₁₃H₁₀ClN₂S₂[MH]⁺ m/z expected=293.0, observed=293.0; HPLC-1=98%.

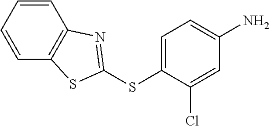 [SMILES: Nc1ccc(Sc2nc3ccccc3s2)c(Cl)c1]

### Example 48

Analog 47: Preparation of 4-(Benzo[d]thiazol-2-ylthio)aniline. 2-Chlorobenzothiazole (2.00 g, 11.8 mmol), 4-aminothiophenol (1.70 g, 13.6 mmol), and potassium carbonate (3.24 g, 23.4 mmol) were stirred together in EtOH (15 mL) for 18 h. The reaction was then diluted with water and the precipitate was filtered, rinsed with water, and collected. Flash chromatographic purification (hexanes:EtOAc gradient) afforded 47 as an off-white solid (2.71 g, 89% yield). ¹H-NMR (300 MHz, d₆-DMSO) δ 7.85-7.91 (m, 1H), 7.78 (d, J=7.7 Hz, 1H), 7.37-7.45 (m, 3H), 7.25-7.34 (m, 1H), 6.66-6.73 (m, 2H), 5.84 (s, 1H); MS (ESI) C₁₃H₁₁N₂S₂[MH]⁺ m/z expected=259.0, observed=259.0; HPLC-1=>99%.

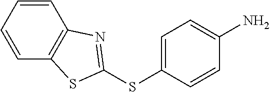 [SMILES: Nc1ccc(Sc2nc3ccccc3s2)cc1]

### Example 49

Analog 48: Preparation of 3,5-Dibromo-2-methoxybenzoic acid. Iodomethane (6.30 mL, 101 mmol), 3,5-dibromosalicylic acid (10.0 g, 33.7 mmol), and K₂CO₃ (14.0 g, 101 mmol) were stirred at R.T. overnight, then at 80° C. for 4 h. The reaction was diluted into water and extracted into DCM. The organics were dried over Na₂SO₄, filtered, and concentrated. The intermediate ester was then stirred overnight with LiOH.H₂O (5.70 g, 136 mmol) in a 3:1:1 mixture of THF:MeOH:H₂O (35 mL). The reaction was diluted with water and acidified with HCl. The precipitate was filtered, washed with water, and dried to afford 48 as a white solid (9.85 g, 94% yield). ¹H-NMR (300 MHz, d₆-DMSO) δ 13.56 (br s, 1H), 8.09 (d, J=2.5 Hz, 1H), 7.83 (d, J=2.5 Hz, 1H), 3.81 (s, 3H); MS (ESI) C₈H₅Br₂O₃[M-H]⁻ m/z expected=308.9, observed=309.1; HPLC-1=>99%.

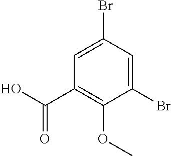

### Example 50

Analog 49: Preparation of 49: 5-Bromo-2-methoxybenzoic acid. 5-Bromosalicylic acid (10.0 g, 46.1 mmol), iodomethane (8.60 mL, 138 mmol), and K₂CO₃ (19.0 g, 137 mmol) were stirred at R.T. overnight, then at 80° C. for 4 h. The reaction was diluted into water and the precipitate was filtered, rinsed with water, and dried. The intermediate ester was then stirred overnight with LiOH.H₂O (7.70 g, 184 mmol) in a 3:1:1 mixture of THF:MeOH:H₂O (45 mL). The reaction was diluted with water and acidified with HCl. The precipitate was filtered, washed with water, and dried to afford 49 as a white solid (8.70 g, 82% yield). ¹H-NMR (300 MHz, d₆-DMSO) δ 12.98 (br s, 1H), 7.72 (d, J=2.6 Hz, 1H), 7.66 (dd, J=8.8, 2.6 Hz, 1H), 7.10 (d, J=8.9 Hz, 1H), 3.81 (s, 3H); MS (ESI) C₈H₆BrO₃ [M-H]⁻ m/z expected=229.0, observed=229.0; HPLC-1=98%.

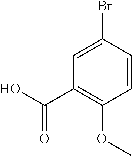

### Example 51

General procedure for the amide coupling step using analog 29 as a representative example: N-(4-(Benzo[d]thiazol-2-ylthio)-3-chlorophenyl)-3,5-dibromo-2-methoxybenzamide. Compound 48 (225 mg, 0.726 mmol) was stirred in SOCl₂ (2 mL) at 60° C. for 1 h, then was concentrated. Anhydrous DCM (5 mL), compound 46 (148 mg, 0.505 mmol), and pyridine (62.0 L, 0.760 mmol) were added and the reaction was stirred at R.T. for 18 h (under Ar). Flash chromatographic purification (hexanes:EtOAc gradient) afforded 29 as a yellow solid (58.6 mg, 20% yield). ¹H-NMR (300 MHz, d₆-DMSO) δ 11.00 (s, 1H), 8.21 (d, J=2.1 Hz, 1H), 8.10 (d, J=2.3 Hz, 1H), 7.97 (t, J=8.3 Hz, 2H), 7.82-7.87 (m, 2H), 7.78 (dd, J=8.6, 2.2 Hz, 1H), 7.47 (td, J=7.7, 1.3 Hz, 1H), 7.32-7.38 (m, 1H), 3.84 (s, 3H); MS (ESI) C₂₁H₁₂Br₂ClN₂O₂S₂ [M-H]⁻ m/z expected=580.8, observed=580.7; HPLC-1=>99%; HPLC-2=>99%.

### Example 52

General procedure for the methoxy deprotection step to give hydroxylated compounds using analog 1 as a representative example: N-(4-(Benzo[d]thiazol-2-ylthio)-3-chlorophenyl)-3,5-dibromo-2-hydroxybenzamide. To a stirring mixture of 29 (51.0 mg, 0.0872 mmol) in anhydrous DCM (5 mL) was added BBr₃ (0.26 mL of 1 M in DCM, 0.26 mmol). The reaction was allowed to stir at R.T. (under Ar) for 18 h and then quenched with MeOH. Flash chromatographic purification (hexanes:EtOAc gradient) afforded 1 as an off-white solid (34.4 mg, 69% yield). ¹H-NMR (300 MHz, d₆-DMSO) δ 11.14 (br s, 1H), 8.19 (dd, J=6.2, 2.2 Hz, 2H), 7.94-8.05 (m, 3H), 7.82-7.89 (m, 2H), 7.47 (td, J=7.7, 1.3 Hz, 1H), 7.33-7.40 (m, 1H); MS (ESI) C₂₀H₁₀Br₂ClN₂O₂S₂ [M-H]⁻ m/z expected=566.8, observed=566.6; HPLC-1=99%; HPLC-2=98%.

### Example 53

Protein Expression and Purification.

E. coli GroEL and GroES purification. E. coli GroEL was expressed from a trc-promoted and Amp(+) resistance marker plasmid in DH5α. E. coli cells. GroES was expressed from a T7-promoted and Amp(+) resistance plasmid in E. coli BL21 (DE3) cells. Transformed colonies were plated onto Ampicillin-treated LB agar and incubated for 24 h at 37° C. Cells were then grown at 37° C. in Ampicillin-treated LB medium until an OD₆₀₀ of 0.5 was reached, then were induced with 0.8 mM IPTG and continued to grow for 2-3 h at 37° C. The cultures were centrifuged at 8,000 rpm and the cell pellets were collected and re-suspended in Buffer A (50 mM Tris-HCl, pH 7.4, and 20 mM NaCl) supplemented with EDTA-free complete protease inhibitor cocktail (Roche). The combined suspension was lysed by sonication, the lysate was centrifuged at 14,000 rpm, and the clarified lysate was passed through a 0.45 μm filter (Millipore).

Anion exchange purification. The filtered lysate was loaded onto a GE HiScale Anion exchange column (Q Sepharose fast flow anion exchange resin) that was equilibrated with 2 column volumes of Buffer A. The loaded column was washed with 4 column volumes of Buffer A containing 30% of Buffer B (50 mM Tris-HCl, pH 7.4, and 1 M NaCl), then bound protein was eluted with a 30-60% gradient elution of Buffer B. Protein-containing fractions, as identified by SDS-PAGE, were collected, spin concentrated using a 10 kDa Amicon Ultra-15 centrifugal filter (EMD Millipore), and dialyzed overnight with 10 kDa SnakeSkin™ dialysis tubing (Thermo Scientific) at 4° C. in 50 mM Tris-HCl, pH 7.4, and 150 mM NaCl solution.

Size exclusion chromatography. The dialyzed protein was loaded onto a Superdex 200 column (HiLoad 26/600, GE) that was equilibrated with 2 column volumes of 50 mM Tris-HCl, pH 7.4, and 150 mM NaCl solution. The loaded column was eluted with 3 column volumes of 50 mM Tris-HCl, pH 7.4, and 150 mM NaCl solution. Protein-containing fractions, as identified by SDS-PAGE, were collected, spin concentrated using a 10 kDa Amicon Ultra-15 centrifugal filter (EMD Millipore), and dialyzed overnight with 10 kDa SnakeSkin™ dialysis tubing (Thermo Scientific) at 4° C. in 50 mM Tris-HCl, pH 7.4, and 150 mM NaCl solution. The final protein concentration was determined by Coomassie Protein Assay Kit (Thermo Scientific). Batches of GroEL and GroES proteins for testing were stored at 4° C. for up to one month then discarded.

Human HSP60 purification. Human HSP60 (mtHSP60) was expressed from a 17-promoted plasmid in Rosetta™ 2 (DE3) in E. coli cells. For human HSP60 purification, a pET21-HSP60 plasmid with an N-terminal octa-Histidine tag was transformed into Rosetta™ 2 (DE3) E. coli cells for over-expression. Cells were grown at 37° C. in LB/ampicillin/chloramphenicol medium until an OD₆₀₀ of 0.5 was reached, then cultures were induced with 0.5 mM IPTG and continued to grow for 2-3 h at 25° C. Cells were centrifuged at 14,000 rpm, and the cell pellet was suspended in 50 mL of lysis buffer composed of 100 mM Tris-HCl, pH 7.7, 10 mM MgSO₄, 1 mM 3-ME, 5% glycerol, 0.1% triton X-100, 1500 Units DNAase, 50 μg/ml lysozyme, and one tablet of EDTA-free complete protease inhibitor cocktail (Roche). Cells were homogenized and passed through a microfluidizer, washing with buffer containing 10 mM Tris-HCl, pH 7.7, 5% glycerol, and 0.1% triton X-100.

1^{st} Nickel column purification and His-tag cleavage: The cell lysate was centrifuged at 14,000 rpm, then the clarified lysate was supplemented with 10 mM imidazole, passed through a 0.2 μm filter, and loaded onto a nickel-agarose resin column that was equilibrated with 2 column volume of 20 mM Tris-HCl pH, 7.7, 5% glycerol, 200 mM NaCl, and 10 mM imidazole. The sample loaded column was washed with 6 column volumes of 50 mM imidazole, then bound HSP60 was eluted with 500 mM imidazole. Fractions that were enriched with the His-tagged mtHSP60 were collected, concentrated, dialyzed at room temperature for 2 h in 4 L of 20 mM Tris-HCl, pH 7.7, 200 mM NaCl and 5% glycerol. Proteolytic cleavage of the His-tag was next performed by addition of His-tagged TEV protease at a 1:10 (w:w) ratio, while dialyzing over night at 4° C. against 4 L of 20 mM Tris-HCl, pH 7.7, 200 mM NaCl, and 5% glycerol buffer.

2^{nd} Nickel column purification: The protein sample was loaded onto a second nickel-agarose resin column that was equilibrated with 20 mM Tris-HCl, pH 7.7, 5% glycerol, 10 mM NaCl, and 10 mM imidazole. With this column, undigested His-tagged mtHSP60 can be separated from digested His-tag removed mtHSP60. The unbound fractions enriched with His-tag cleaved mtHSP60 were collected, and anion exchange chromatography was performed on the same day.

Anion exchange purification of His-tag removed mtHSP60: The protein sample was next loaded onto an anion-exchange column that was equilibrated with 20 mM Tris-HCl, pH 7.7, and 5% glycerol. Bound proteins were eluted from the column with a linear gradient of 100-400 mM NaCl. Fractions enriched with mtHSP60 were collected, concentrated, and dialyzed in storage buffer (20 mM Tris-HCl, pH 7.7, 300 mM NaCl, 5% glycerol, and 10 mM MgCl₂) using 10 kDa SnakeSkin™ dialysis tubing (Thermo Scientific). The concentration of protein was determined by Coomassie Protein Assay Kit (Thermo Scientific). Batches of HSP60 protein for testing were stored at 4° C. for up to two weeks, then discarded.

Human HSP10 purification: Human HSP10 (mtHSP10) was expressed from a 17-promoted (pET3a-HSP10) plasmid in Rosetta™ 2 (DE3) pLysS cells. Cells were grown at 37° C. in LB/kanamycin/chloramphenicol medium until an OD₆₀₀ of 0.5 was reached, then were induced with 0.5 mM IPTG and continued to grow for 2-3 h at 37° C. The culture was centrifuged at 14,000 rpm, and the cell pellet was re-suspended in Buffer A (50 mM sodium acetate, pH 4.5, and 20 mM NaCl), supplemented with EDTA-free complete protease inhibitor cocktail (Roche®) and lysed by sonication. Clarified cell lysate was loaded on a cation exchange column (SP Sepharose fast flow resin, GE) and eluted with a linear NaCl gradient using Buffer B (50 mM sodium acetate, pH 4.5, and 1 M NaCl). Fractions containing HSP10 were concentrated, dialyzed with storage buffer (50 mM Tris-HCl, pH 7.4, and 300 mM NaCl) using 10 kDa SnakeSkin™ dialysis tubing (Thermo Scientific) and re-purified on a Superdex 200 column (HiLoad 26/600, GE) in storage buffer. The concentration of protein was determined by Coomassie Protein Assay Kit (Thermo Scientific). Protein was stored at 4° C. in 50 mM Tris-HCl, pH 7.4, and 150 mM NaCl. Batches of HSP10 protein for testing were stored at 4° C. for up to three weeks, then discarded.

### Example 54

Evaluation of Compounds in GroEL/ES and HSP60/10-Mediated dMDH Refolding Assays.

Reagent preparation: For these assays, four primary reagent stocks were prepared: 1) GroEUES-dMDH or HSP60/10-dMDH binary complex stock; 2) ATP initiation stock; 3) EDTA quench stock; 4) MDH enzymatic assay stock. Denatured MDH (dMDH) was prepared by 2-fold dilution of MDH (5 mg/ml, soluble pig heart MDH from Roche, product #10127248001) with denaturant buffer (7 M guanidine-HCl, 200 mM Tris, pH 7.4, and 50 mM DTT). MDH was completely denatured by incubating at room temperature for 45 min. The binary complex solutions were prepared by slowly adding the dMDH stock to a stirring stock with GroEL (or HSP60) in folding buffer (50 mM Tris-HCl, pH 7.4, 50 mM KCl, 10 mM MgCl₂, and 1 mM DTT), followed by addition of GroES (or HSP10). The binary complex stocks were prepared immediately prior to dispensing into the assay plates and had final protein concentrations of 83.3 nM GroEL (Mr 800 kDa) or HSP60 (Mr 400 kDa), 100 nM GroES or HSP10 (Mr 70 kDa), and 20 nM dMDH in folding buffer. For the ATP initiation stock, ATP solid was diluted into folding buffer to a final concentration of 2.5 mM. Quench solution contained 600 mM EDTA (pH 8.0). The MDH enzymatic assay stock consisted of 20 mM sodium mesoxalate and 2.4 mM NADH in reaction buffer (50 mM Tris-HCl, pH 7.4, 50 mM KCl, and 1 mM DTT).

Assay Protocol: First, 30 μL aliquots of the GroEL/ES-dMDH or HSP60/10-dMDH binary complex stocks were dispensed into clear, 384-well polystyrene plates. Next, 0.5 μL of the compound stocks (10 mM to 4.6 μM, 3-fold dilutions series in DMSO) were added by pin-transfer (V&P Scientific). The chaperonin-mediated refolding cycles were initiated by addition of 20 μL of ATP stock (reagent concentrations during refolding cycle: 50 nM GroEL or HSP60, 60 nM GroES or HSP10, 12 nM dMDH, 1 mM ATP, and compounds of 100 μM to 46 nM, 3-fold dilution series). The refolding reactions were incubated at 37° C. The incubation time was determined from refolding time-course control experiments until they reached ˜90% completion of refolding cycle—generally ˜20-40 min for GroEL/ES, and ˜40-60 min for HSP60/10). Next, the assay was quenched by addition of 10 μL of the EDTA to final concentration of 100 mM. Enzymatic activity of the refolded MDH was initiated by addition of 20 μL MDH enzymatic assay stock (20 mM sodium mesoxalate and 2.4 mM NADH in reaction buffer, 50 mM Tris pH 7.4, 50 mM KCl, 1 mM DTT), and followed by measuring the NADH absorbance in each well at 340 nm using a Molecular Devices SpectraMax Plus384 microplate reader (NADH absorbs at 340 nm, while NAD⁺ does not). A_{340 nm} measurements were recorded at 0.5 minutes (start point) and at successive time points until the amount of NADH consumed reached ˜90% (end point, generally between 20-35 minutes). The differences between the start and end point A₃₄₀ values were used to calculate the % inhibition of the GroEL/ES or HSP60/10 machinery by the compounds. IC₅₀ values for the test compounds were obtained by plotting the % inhibition results in GraphPad Prism 6 and analyzing by non-linear regression using the log (inhibitor) vs. response (variable slope) equation. Results presented represent the averages of IC₅₀ values obtained from at least quadriplicate (duplicate of duplicate) replicates.

### Example 55

Counter-Screening Compounds for Inhibition of Native MDH Enzymatic Activity.

Reagent Preparations & Assay Protocol: This assay was performed as described above for the GroEL/ES-dMDH refolding assay; however, the assay protocol differed in the sequence of compound addition to the assay plates. The refolding reactions were allowed to proceed for 45 min at 37° C. in the absence of test compounds (complete refolding of MDH occurs), then quenched with the EDTA stock. Compounds were then pin-transferred into the plates after the EDTA quenching step; thus, compounds effects are only possible by inhibiting the fully-refolded MDH reporter substrate. Next enzymatic activity of the refolded MDH was initiated by addition of 20 μL MDH enzymatic assay stock (20 mM sodium mesoxalate and 2.4 mM NADH in reaction buffer, 50 mM Tris pH 7.4, 50 mM KCl, 1 mM DTT), and followed by measuring the NADH absorbance in each well at 340 nm using a Molecular Devices SpectraMax Plus384 microplate reader (NADH absorbs at 340 nm, while NAD⁺ does not). A_{340 nm} measurements were recorded at 0.5 minutes (start point) and at successive time points until the amount of NADH consumed reached ˜90% (end point, generally between 20-35 minutes). Compounds were tested in 8-point, 3-fold dilution series (62.5 μM to 29 nM during the reporter reaction) in clear, flat-bottom 384-well microtiter plates. DMSO was used as a negative control, and previously discovered native MDH inhibitors were used as positive controls. IC₅₀ values for the test compounds were obtained by plotting the % inhibition results in GraphPad Prism 6 and analyzing by non-linear regression using the log (inhibitor) vs. response (variable slope) equation. Results presented represent the averages of IC₅₀ values obtained from at least quadriplicate (duplicate of duplicate) replicates.

### Example 56

Evaluation of Compounds in GroEL/ES-Mediated dRho Refolding Assay.

Reagent preparation: For this assay, five primary reagent stocks were prepared: 1) GroEUES-dRho binary complex stock; 2) ATP initiation stock; 3) thiocyanate enzymatic assay stock; 4) formaldehyde quench stock; 5) ferric nitrate reporter stock. Denatured rhodanese (dRho) was prepared by 3-fold dilution of rhodanese (Roche product #R1756, diluted to 10 mg/mL with H₂O) with denaturant buffer (12 M Urea, 50 mM Tris-HCl, pH 7.4, and 10 mM DTT). Rhodanese was completely denatured by incubating at room temperature for 45 min. The binary complex solution was prepared by slowly adding the dRho stock to a stirring stock of concentrated GroEL in modified folding buffer (50 mM Tris-HCl, pH 7.4, 50 mM KCl, 10 mM MgCl₂, 5 mM Na₂S₂O₃, and 1 mM DTT). The solution was centrifuged at 16,000×g for 5 minutes, and the supernatant was collected and added to a solution of GroES in modified folding buffer to give final protein concentrations of 100 nM GroEL, 120 nM GroES, and 80 nM dRho. The binary complex stock was prepared immediately prior to use. For the ATP initiation stock, ATP solid was diluted into modified folding buffer to a final concentration of 2.0 mM. The thiocyanate enzymatic assay stock was prepared to contain 70 mM KH₂PO₄, 80 mM KCN, and 80 mM Na₂S₂O₃ in water. The formaldehyde quench solution contained 30% formaldehyde in water. The ferric nitrate reporter stock contained 8.5% w/v Fe(NO₃)₃ and 11.3% v/v HNO₃ in water.

Assay Protocol: First, 10 μL aliquots of the GroEL/ES-dRho complex stock were dispensed into clear, 384-well polystyrene plates. Next, 0.5 μL of the compound stocks (10 mM to 4.6 μM, 3-fold dilutions in DMSO) were added by pin-transfer. The chaperonin-mediated refolding cycle was initiated by addition of 10 μL of ATP stock (reagent concentrations during refolding cycle: 50 nM GroEL, 60 nM GroES, 40 nM dRho, 1 mM ATP, and compounds of 250 μM to 114 nM, 3-fold dilution series). After incubating for 45 minutes at 37° C. for the refolding cycle, 30 μL of the thiocyanate enzymatic assay stock was added and incubated for 60 min at R.T. for the refolded rhodenase enzymatic reporter reaction. The reporter reaction was quenched by adding 10 μL of the formaldehyde quench stock, and then 40 μL of the ferric nitrate reporter stock was added to quantify the amount of thiocyanate produced during the enzymatic reporter reaction, which is proportional to the amount of dRho refolded by GroEL/ES. After incubating at R.T. for 15 min, the absorbance by Fe(SCN)₃ was measured at 460 nm using a Molecular Devices SpectraMax Plus384 microplate reader. A second set of baseline control plates were prepared analogously, but without GroEL/ES-dRho protein binary solution, to correct for possible interference from compound absorbance or turbidity. IC₅₀ values for the test compounds were obtained by plotting the A₄₆₀ results in GraphPad Prism 6 and analyzing by non-linear regression using the log(inhibitor) vs. response (variable slope) equation. Results presented represent the averages of IC₅₀ values obtained from at least quadriplicate (duplicate of duplicate) replicates.

### Example 57

Counter-Screening Compounds for Inhibition of Native Rhodanese Enzymatic Activity.

Reagent Preparations & Assay Protocol: Reagents were identical to those used in the GroEUES-dRho refolding assay described above; however, the assay protocol differed in the sequence of compound addition to the wells. Compounds were pin-transferred after the 60 minute incubation for the refolding cycle, but prior to the addition of the thiocyanate enzymatic assay stock. Thus, the refolding reactions were allowed to proceed for 60 min at 37° C. in the absence of test compounds, but the enzymatic activity of the refolded rhodanese reporter enzyme was monitored in the presence of test compounds (inhibitor concentration range during the enzymatic reporter reaction is 100 M to 46 nM-3-fold dilutions). IC₅₀ values for the rhodenase reporter enzyme were determined as described above. Results presented represent the averages of IC₅₀ values obtained from at least quadriplicate (duplicate of duplicate) replicates.

### Example 58

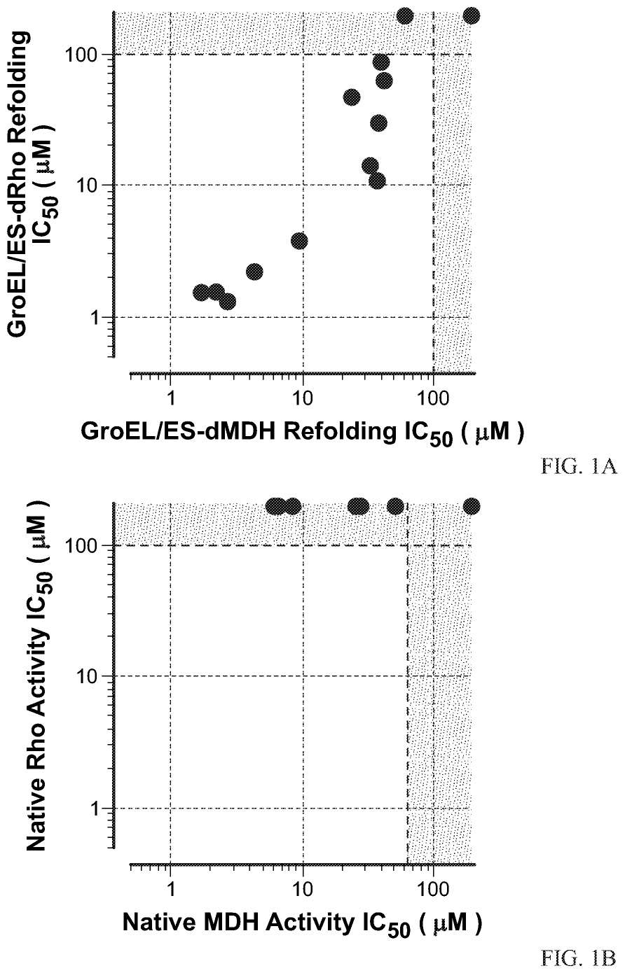

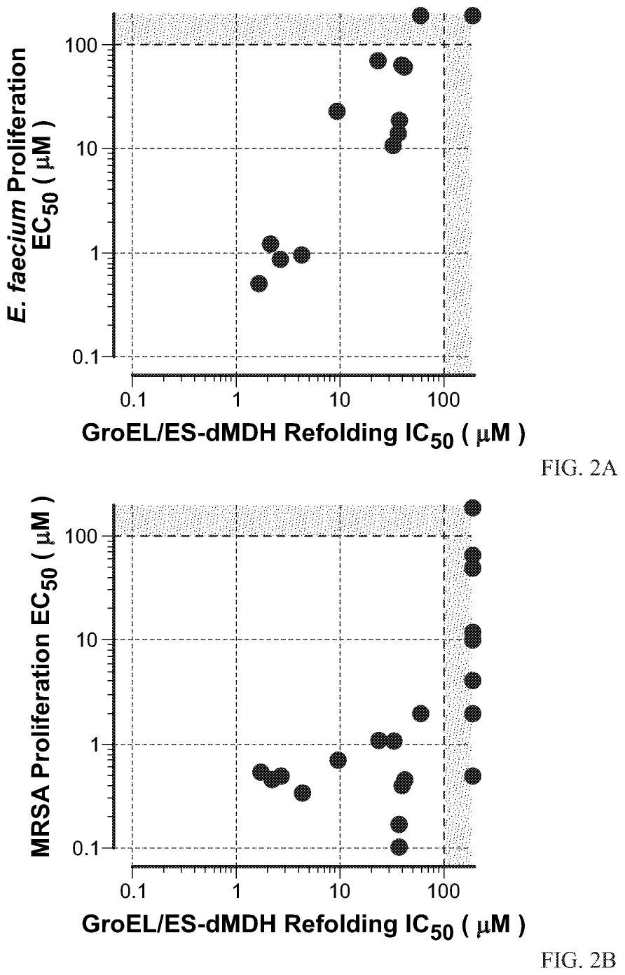

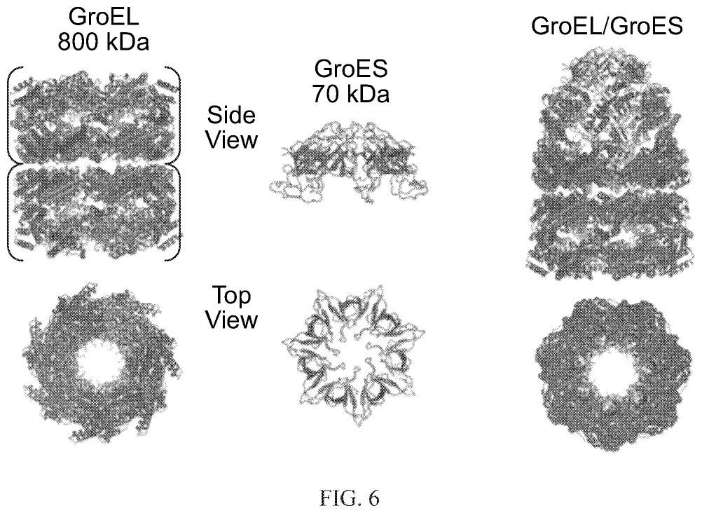

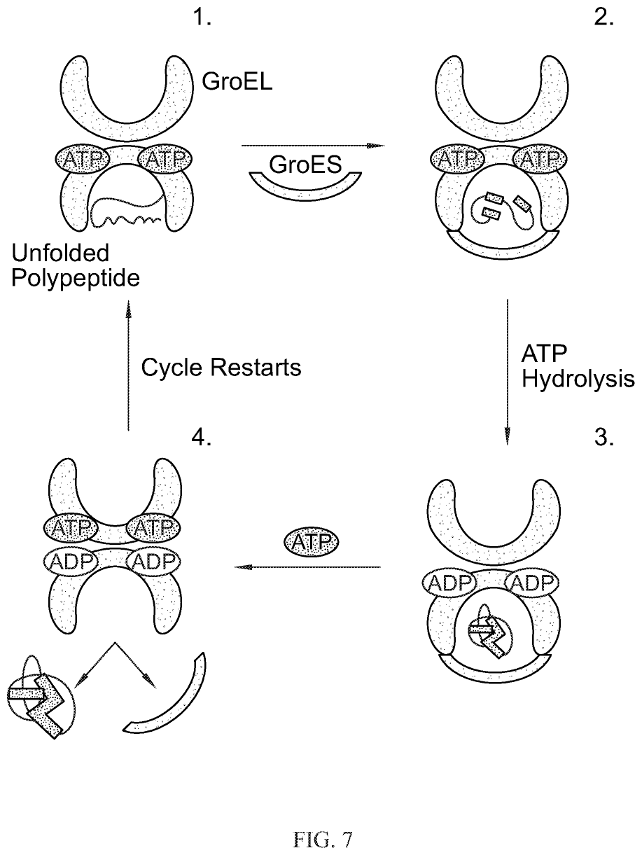

After generating the compound library, we next employed a series of well-established biochemical assays to evaluate compound inhibitory effects against the GroEUES chaperonin system (See FIG. 6 and FIG. 7). We used E. coli GroEUES as the surrogate chaperonin system for refolding of the reporter enzymes malate dehydrogenase (MDH) and rhodanese (Rho). Briefly, binary complexes are formed between GroEL and denatured MDH or Rho, which are then refolded to their native states upon addition of ATP and the GroES co-chaperonin. Enzymatic activities of refolded native MDH or Rho act as coupled reporters of GroEUES refolding functions since in the presence of chaperonin inhibitors, no functional reporter enzymes are generated. Inhibition IC₅₀ results for testing of compounds in these two refolding assays are compiled in Table 1, and visually presented in the correlation plot in FIG. 1A. The log-transformations of IC₅₀ results and standard deviations are presented in Table 2. As seen in FIG. 2A, there is a strong correlation between compounds inhibiting in the GroEUES-dMDH and dRho refolding assays (Spearman correlation coefficient comparing log(IC₅₀) values in each assay is 0.9663, p<0.0001), suggesting they are acting on-target against the chaperonin system. In general, binding is hypothesized as the R¹-benzothiazole coupled with the R³-hydroxyl may be required for potent inhibition. Halogenation at the R²-position (Cl) and R⁴/R⁵ positions (Br) further increases inhibitor potency, possibly through a combination of increased hydrophobic interactions of the halides themselves, coupled with the electron-withdrawing capability of the bromines lowering the pKₐ of the salicylate hydroxyl, which could enhance polar interactions within the binding sites. Interestingly, we found that closantel and rafoxanide were potent GroEUES inhibitors, although not designed for this use.

To further support on-target effects, we evaluated inhibitors against the native MDH and Rho enzymes to identify false-positives that simply inhibit the reporter reactions of the coupled refolding assays. While some compounds were found to inhibit native MDH (e.g. 1, 5, closantel, and to lesser extents, 9, 13, and rafoxanide), none of the analogs were found to inhibit native Rho enzymatic activity (Table 1 and FIG. 1B). While these results further support that inhibitors are targeting the chaperonin-mediated folding cycle, they also indicate that selectivity issues may be a liability that future studies would need to address. Hearteningly, though, a few analogs (e.g. compound 2, 3, 6, and 7) are moderate to potent inhibitors in the GroEL/ES-mediated refolding assays yet remained inactive against the reporter enzymes, suggesting selectivity concerns are not insurmountable.

TABLE 1
Compilation of IC₅₀ results for compounds tested in the GroEL/ES-mediated
dMDH and dRho refolding assays, and the native MDH and Rho reporter counter-screens.

| 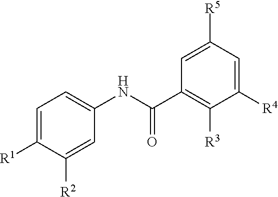 | | | | | Biochemical Assay IC₅₀ (μM) | | | | |
|---|---|---|---|---|---|---|---|---|---|
| Compound Substituents & Substructures | | | | | Compound | Native Rho | Native MDH | GroEL/ES-dRho | GroEL/ES-dMDH |
| R¹ | R² | R³ | R⁴ | R⁵ | #/Name | Reporter | Reporter | Refolding | refolding |
| | | | | | Closantel | >100 | 6.1 | 1.5 | 2.2 |
| | | | | | Rafoxanide | >100 | 25 | 2.2 | 4.3 |
| 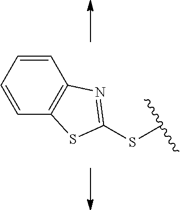 [SMILES: *Sc1nc2ccccc2s1] | Cl Cl Cl Cl H H H H | OH OH OH OH OH OH OH OH | Br Br H H Br Br H H | Br H Br H Br H Br H | 1 2 3 4 5 6 7 8 | >100 >100 >100 >100 >100 >100 >100 >100 | 6.8 >63 >63 >63 8.4 >63 >63 >63 | 1.5 3.8 11 63 1.3 14 30 87 | 1.7 9.5 37 42 2.7 33 38 40 |
| H | Cl | OH | Br | Br | 9 | >100 | 27 | 47 | 24 |
| H | Cl | OH | Br | H | 10 | >100 | >63 | >100 | >100 |
| H | Cl | OH | H | Br | 11 | >100 | >63 | >100 | >100 |
| H | Cl | OH | H | H | 12 | >100 | >63 | >100 | >100 |
| H | H | OH | Br | Br | 13 | >100 | 51 | >100 | 61 |
| H | H | OH | Br | H | 14 | >100 | >63 | >100 | >100 |
| H | H | OH | H | Br | 15 | >100 | >63 | >100 | >100 |
| H | H | OH | H | H | 16 | >100 | >63 | >100 | >100 |
| 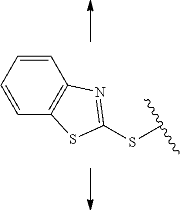 [SMILES: *Sc1nc2ccccc2s1] | Cl Cl Cl H H H | H H H H H H | Br Br (H) H Br Br (H) H | Br H (Br) H Br H (Br) H | 17 18 19 20 21 22 | >100 >100 >100 >100 >100 >100 | >63 >63 >63 >63 >63 >63 | >100 >100 >100 >100 >100 >100 | >100 >100 >100 >100 >100 >100 |
| H | Cl | H | Br | Br | 23 | >100 | >63 | >100 | >100 |
| H | Cl | H | Br (H) | H (Br) | 24 | >100 | >63 | >100 | >100 |
| H | Cl | H | H | H | 25 | >100 | >63 | >100 | >100 |
| H | H | H | Br | Br | 26 | >100 | >63 | >100 | >100 |
| H | H | H | Br (H) | H (Br) | 27 | >100 | >63 | >100 | >100 |
| H | H | H | H | H | 28 | >100 | >63 | >100 | >100 |
| 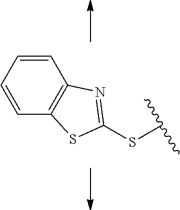 [SMILES: *Sc1nc2ccccc2s1] | Cl Cl Cl Cl H H H H | OCH₃ OCH₃ OCH₃ OCH₃ OCH₃ OCH₃ OCH₃ OCH₃ | Br Br H H Br Br H H | Br H Br H Br H Br H | 29 30 31 32 33 34 35 36 | >100 >100 >100 >100 >100 >100 >100 >100 | >63 >63 >63 >63 >63 >63 >63 >63 | >100 >100 >100 >100 >100 >100 >100 >100 | >100 >100 >100 >100 >100 >100 >100 >100 |
| H | Cl | OCH₃ | Br | Br | 37 | >100 | >63 | >100 | >100 |
| H | Cl | OCH₃ | Br | H | 38 | >100 | >63 | >100 | >100 |
| H | Cl | OCH₃ | H | Br | 39 | >100 | >63 | >100 | >100 |
| H | Cl | OCH₃ | H | H | 40 | >100 | >63 | >100 | >100 |
| H | H | OCH₃ | Br | Br | 41 | >100 | >63 | >100 | >100 |
| H | H | OCH₃ | Br | H | 42 | >100 | >63 | >100 | >100 |
| H | H | OCH₃ | H | Br | 43 | >100 | >63 | >100 | >100 |
| H | H | OCH₃ | H | H | 44 | >100 | >63 | >100 | >100 |
| | | | | | Vancomycin | >100 | >63 | >100 | >100 |
| | | | | | Daptomycin | >100 | >63 | >100 | >100 |
| | | | | | Ampicillin | >100 | >63 | >100 | >100 |
| | | | | | Rifampicin | >100 | >63 | >100 | >100 |

TABLE 2
Compilation of log(IC₅₀) results and standard deviations for compounds tested in the GroEL/ES-mediated dMDH and dRho
refolding assays, and the native MDH and Rho reporter counter-screens.

| 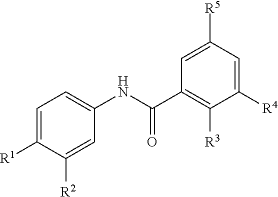 | | | | | Biochemical Assay | | | | |
|---|---|---|---|---|---|---|---|---|---|
| Compound Subtituents & Substructures | | | | | Compound | Native Rho | Native MDH | GroEL/ES-dRho | GroEL/ES-dMDH |
| R¹ | R² | R³ | R⁴ | R⁵ | #/Name | Reporter | Reporter | Refolding | Refolding |
| | | | | | Closantel | >2 | 0.79 ± 0.17 | 0.19 ± 0.10 | 0.35 ± 0.19 |
| | | | | | Rafoxanide | >2 | 1.40 ± 0.34 | 0.34 ± 0.11 | 0.64 ± 0.27 |
| 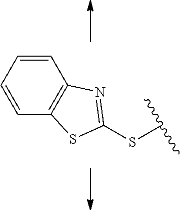 [SMILES: *Sc1nc2ccccc2s1] | Cl Cl Cl Cl H H H H | OH OH OH OH OH OH OH OH | Br Br H H Br Br H H | Br H Br H Br H Br H | 1 2 3 4 5 6 7 8 | >2 >2 >2 >2 >2 >2 >2 >2 | 0.83 ± 0.25 >1.8 >1.8 >1.8 0.92 ± 0.21 >1.8 >1.8 >1.8 | 0.19 ± 0.09 0.58 ± 0.15 1.04 ± 0.38 1.80 ± 0.27 0.12 ± 0.37 1.14 ± 0.02 1.48 ± 0.11 1.94 ± 0.23 | 0.23 ± 0.28 0.98 ± 0.23 1.57 ± 0.35 1.62 ± 0.32 0.43 ± 0.19 1.52 ± 0.21 1.58 ± 0.34 1.60 ± 0.26 |
| H | Cl | OH | Br | Br | 9 | >2 | 1.40 ± 0.17 | 1.67 ± 0.04 | 1.38 ± 0.15 |
| H | Cl | OH | Br | H | 10 | >2 | >1.8 | >2 | >2 |
| H | Cl | OH | H | Br | 11 | >2 | >1.8 | >2 | >2 |
| H | Cl | OH | H | H | 12 | >2 | >1.8 | >2 | >2 |
| H | H | OH | Br | Br | 13 | >2 | 1.70 ± 0.19 | >2 | 1.79 ± 0.10 |
| H | H | OH | Br | H | 14 | >2 | >1.8 | >2 | >2 |
| H | H | OH | H | Br | 15 | >2 | >1.8 | >2 | >2 |
| H | H | OH | H | H | 16 | >2 | >1.8 | >2 | >2 |
| 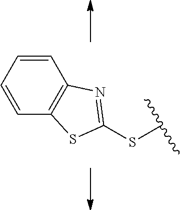 [SMILES: *Sc1nc2ccccc2s1] | Cl Cl Cl H H H | H H H H H H | Br Br (H) H Br Br (H) H | Br H (Br) H Br H (Br) H | 17 18 19 20 21 22 | >2 >2 >2 >2 >2 >2 | >1.8 >1.8 >1.8 >1.8 >1.8 >1.8 | >2 >2 >2 >2 >2 >2 | >2 >2 >2 >2 >2 >2 |
| H | Cl | H | Br | Br | 23 | >2 | >1.8 | >2 | >2 |
| H | Cl | H | Br (H) | H (Br) | 24 | >2 | >1.8 | >2 | >2 |
| H | Cl | H | H | H | 25 | >2 | >1.8 | >2 | >2 |
| H | H | H | Br | Br | 26 | >2 | >1.8 | >2 | >2 |
| H | H | H | Br (H) | H (Br) | 27 | >2 | >1.8 | >2 | >2 |
| H | H | H | H | H | 28 | >2 | >1.8 | >2 | >2 |
| 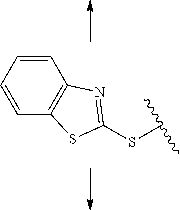 [SMILES: *Sc1nc2ccccc2s1] | Cl Cl Cl Cl H H H H | OCH₃ OCH₃ OCH₃ OCH₃ OCH₃ OCH₃ OCH₃ OCH₃ | Br Br H H Br Br H H | Br H Br H Br H Br H | 29 30 31 32 33 34 35 36 | >2 >2 >2 >2 >2 >2 >2 >2 | >1.8 >1.8 >1.8 >1.8 >1.8 >1.8 >1.8 >1.8 | >2 >2 >2 >2 >2 >2 >2 >2 | >2 >2 >2 >2 >2 >2 >2 >2 |
| H | Cl | OCH₃ | Br | Br | 37 | >2 | >1.8 | >2 | >2 |
| H | Cl | OCH₃ | Br | H | 38 | >2 | >1.8 | >2 | >2 |
| H | Cl | OCH₃ | H | Br | 39 | >2 | >1.8 | >2 | >2 |
| H | Cl | OCH₃ | H | H | 40 | >2 | >1.8 | >2 | >2 |
| H | H | OCH₃ | Br | Br | 41 | >2 | >1.8 | >2 | >2 |
| H | H | OCH₃ | Br | H | 42 | >2 | >1.8 | >2 | >2 |
| H | H | OCH₃ | H | Br | 43 | >2 | >1.8 | >2 | >2 |
| H | H | OCH₃ | H | H | 44 | >2 | >1.8 | >2 | >2 |
| | | | | | Vancomycin | >2 | >1.8 | >2 | >2 |
| | | | | | Daptomycin | >2 | >1.8 | >2 | >2 |
| | | | | | Amipicillin | >2 | >1.8 | >2 | >2 |
| | | | | | Rifampicin | >2 | >1.8 | >2 | >2 |

### Example 59

Evaluation of Compounds for Inhibition of Bacterial Cell Proliferation.

Bacterial Strains: Enterococcus faecium—(Orla-Jensen) Schleifer and Kilpper-Balz strain NCTC 7171 (ATCC 19434); Drug sensitive Staphylococcus aureus—Rosenbranch strain Seattle 1945 (ATCC 25923); Methicillin resistant S. aureus (MRSA)—Rosenbach strain HPV107 (ATCC BAA-44); Klebsiella Pneumonia—(Schroeter) Trevisan strain NCTC 9633 (ATCC 13883); Acinetobacter baumannii—Bouvet and Grimont strain 2208 (ATCC 19606); Pseudomonas aeruginosa—(Schroeter) Migula strain NCTC 10332 (ATCC 10145); Enterobacter cloacae—E. cloacae, subsp. cloacae (Jordan) Hormaeche and Edwards strain CDC 442-68 (ATCC 13047).

Growth Media: All bacteria were grown in Brain Heart Infusion (BHI) broth/agar (Becton, Dickinson and Company). All liquid cultures were grown in BHI media supplemented with 25 mg/L Ca²⁺ and 12.5 mg/L Mg²⁺ to mimic free physiological concentrations of these cations. A 10 mg/mL Ca²⁺ stock solution was prepared by dissolving 3.68 g of CaCl₂.2H₂O in 100 mL of deionized water, and a 10 mg/mL Mg²⁺ stock solution was prepared by dissolving 8.36 g of MgCl₂.6H₂O in 100 mL deionized water. Both stock solutions were filter-sterilized using 0.2 μm pore size cellulose-acetate filters. 2.5 mL of the sterile 10 mg/mL Ca²⁺ stock and 1.25 mL of the sterile 10 mg/mL Mg²⁺ stock solutions were added per 1 L of autoclaved BHI medium to obtain 25 mg/L Ca²⁺ and 12.5 mg/L Mg²⁺ ions, respectively.

General Assay Protocol: Stock bacterial cultures were streaked onto BHI agar plates and grown overnight at 37° C. Fresh aliquots of broth were inoculated with single bacterial colonies and the cultures were grown overnight at 37° C. with shaking (240 rpm) in cation adjusted BHI media. The following morning, the overnight cultures were sub-cultured (1:5 dilution) into fresh aliquots of cation adjusted BHI media and grown at 37° C. for 1-2 hours with shaking. After 2 h, cultures were diluted into fresh media to achieve final OD₆₀₀ readings of 0.017. Aliquots of these diluted cultures (30 μL) were added to clear, flat-bottom, 384-well polystyrene plates that were stamped with 0.5 μL of test compounds in 20 μL media. Since the Gram-negative bacteria were resistant to the previously developed inhibitors, compounds were first tested at single concentrations of 100 μM (duplicate of duplicate experiments) to increase throughput in the initial screening efforts. Compounds that failed to inhibit bacterial proliferation by >50% in the four replicates were assigned EC₅₀ values >100 μM. Those that did inhibit bacterial proliferation by >50% were further evaluated in dose-response format where the inhibitor concentration range during the proliferation assay was 100 μM to 46 nM (3-fold dilution series). Since many compounds were found to inhibit the S. aureus and MRSA strains, all compounds were tested in dose-response format against these bacteria in at least quadruplicate (duplicate of duplicate) replicates. Plates were sealed with “Breathe Easy” oxygen permeable membranes (Diversified Biotech) and left to incubate at 37° C. without shaking (stagnant assay). OD_{600 nm} readings were taken at either 6-8 h (S. aureus, MRSA, K. pneumonia, E. cloacae, and P. aeruginosa) or 24 h (E. faecium and A. baumannii). A second set of baseline control plates were prepared analogously, but without any bacteria added, to correct for possible compound absorbance and/or precipitation. Plates were then read at 600 nm using a Molecular Devices SpectraMax Plus384 microplate reader. EC₅₀ values for the test compounds were obtained by plotting the OD₆₀₀ results in GraphPad Prism 6 and analyzing by non-linear regression using the log(inhibitor) vs. response (variable slope) equation. Results presented represent the averages of EC₅₀ values obtained from at quadriplicate (duplicate of duplicate) replicates.

### Example 60

Determining the Antibacterial Effects of Compounds Against the ESKAPE Pathogens.

We next evaluated compounds for antibacterial efficacy against the ESKAPE pathogens in liquid media culture. Inhibition EC₅₀ results for testing of compounds in these bacterial proliferation assays are shown in Table 3 and Table 4. In general, this series of analogs demonstrated decreased efficacy against the Gram-negative bacteria (K. pneumonia, A. baumannii, P. aeruginosa, and E. cloacae), likely owing to drug efflux and/or impermeability to the lipopolysaccharide (LPS) outer membranes of Gram-negative bacteria. However, with regards to A. baumannii, notable exceptions are compounds 2 and 6, which exhibit EC₅₀ values of 2.9 and 12 μM, respectively. As previously observed with compound 1, several analogs retained antibacterial efficacy against the Gram-positive bacteria, E. faecium and S. aureus, although they were surprisingly more effective against S. aureus. However, it is noted that incorporation of the benzothiazole substructure at the R¹ position increased the potency of the inhibitors, which may support on-target effects since these analogs are able to inhibit GroEL/ES-mediated folding functions (See FIG. 7). Importantly, these analogs are all equipotent against the MRSA strain that we evaluated compounds against (ATCC #BAA-44, HPV107 designation, isolated from a hospital in Lisbon, Portugal).

When comparing the EC₅₀ results of this series of compounds against E.faecium bacteria with the IC₅₀ values obtained in the GroEL/ES-dMDH refolding assay (FIG. 2A), we note an almost linear correlation (Spearman correlation coefficient comparing log(I/EC₅₀) values in each assay is 0.9628, p<0.0001), which may indicate on-target effects against GroEL/ES driving antibacterial activity. When performing the same comparison with MRSA bacteria (FIG. 2B), although a trend is evident between antibacterial efficacy and GroEL/ES inhibition MRSA (Spearman correlation coefficient comparing log(I/EC₅₀) values in each assay is 0.8042, p<0.0001), several compounds that are not GroEL/ES inhibitors still remain effective against bacteria (e.g. 10-16). This finding could be a result of S. aureus GroEL/ES functioning differently than the E. coli GroEL/ES chaperonin system, which we use as a surrogate in these studies.

TABLE 3
Compilation of EC₅₀ results for compounds tested in the various bacterial proliferation assays.

| 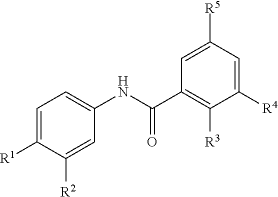 | | | | | Bacterial Proliferation EC₅₀ (μM) | | | | | | | |
|---|---|---|---|---|---|---|---|---|---|---|---|---|
| Compound Substitutents & Substructures | | | | | Compound | E. | S. aureus | | K. | A. | P. | E. |
| R¹ | R² | R³ | R⁴ | R⁵ | #/Name | faecium | Sensitive | Resistant | pneumoniae | baumannii | aeruginosa | cloacae |
| | | | | | Closantel | 1.2 | 0.47 | 0.47 | >100 | >100 | >100 | >100 |
| | | | | | Rafoxanide | 0.99 | 0.32 | 0.34 | >100 | 31 | >100 | 40 |
| 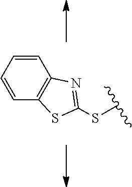 [SMILES: *Sc1nc2ccccc2s1] | Cl Cl Cl Cl H H H H | OH OH OH OH OH OH OH OH | Br Br H H Br Br H H | Br H Br H Br H Br H | 1 2 3 4 5 6 7 8 | 0.52 24 15 64 0.88 11 19 67 | 0.36 0.45 0.12 0.31 0.44 0.76 0.12 0.20 | 0.55 0.71 0.17 0.45 0.52 1.1 0.14 0.41 | >100 >100 >100 >100 >100 >100 >100 >100 | 66 2.9 >100 >100 >100 12 >100 >100 | >100 >100 >100 >100 >100 >100 >100 >100 | >100 >100 >100 >100 >100 >100 >100 >100 |
| H | Cl | OH | Br | Br | 9 | 73 | 0.66 | 1.1 | 95 | 46 | >100 | 78 |
| H | Cl | OH | Br | H | 10 | >100 | 1.3 | 2.0 | >100 | >100 | >100 | >100 |
| H | Cl | OH | H | Br | 11 | >100 | 0.46 | 0.53 | >100 | >100 | >100 | >100 |
| H | Cl | OH | H | H | 12 | >100 | 3.1 | 4.2 | >100 | >100 | >100 | >100 |
| H | H | OH | Br | Br | 13 | >100 | 1.2 | 2.0 | >100 | >100 | >100 | >100 |
| H | H | OH | Br | H | 14 | >100 | 6.4 | 10 | >100 | >100 | >100 | >100 |
| H | H | OH | H | Br | 15 | >100 | 2.8 | 4.1 | 78 | >100 | >100 | >100 |
| H | H | OH | H | H | 16 | >100 | 28 | 51 | >100 | >100 | >100 | >100 |
| 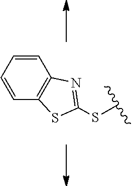 [SMILES: *Sc1nc2ccccc2s1] | Cl Cl Cl H H H | H H H H H H | Br Br (H) H Br Br (H) H | Br H (Br) H Br H (Br) H | 17 18 19 20 21 22 | >100 >100 >100 >100 >100 >100 | >100 >100 43 >100 >100 >100 | >100 >100 >100 >100 >100 >100 | >100 >100 >100 >100 >100 >100 | >100 >100 >100 >100 >100 >100 | >100 >100 >100 >100 >100 >100 | >100 >100 >100 >100 >100 >100 |
| H | Cl | H | Br | Br | 23 | >100 | 14 | 12 | >100 | >100 | >100 | >100 |
| H | Cl | H | Br (H) | H (Br) | 24 | >100 | 47 | 68 | >100 | >100 | >100 | >100 |
| H | Cl | H | H | H | 25 | >100 | >100 | >100 | >100 | >100 | >100 | >100 |
| H | H | H | Br | Br | 26 | >100 | >100 | >100 | >100 | >100 | >100 | >100 |
| H | H | H | Br (H) | H (Br) | 27 | >100 | >100 | >100 | >100 | >100 | >100 | >100 |
| H | H | H | H | H | 28 | >100 | >100 | >100 | >100 | >100 | >100 | >100 |
| 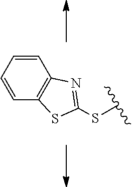 [SMILES: *Sc1nc2ccccc2s1] | Cl Cl Cl Cl H H H H | OCH₃ OCH₃ OCH₃ OCH₃ OCH₃ OCH₃ OCH₃ OCH₃ | Br Br H H Br Br H H | Br H Br H Br H Br H | 29 30 31 32 33 34 35 36 | >100 >100 >100 >100 >100 >100 >100 >100 | >100 >100 >100 >100 >100 >100 >100 >100 | >100 >100 >100 >100 >100 >100 >100 >100 | >100 >100 >100 >100 >100 >100 >100 >100 | >100 >100 >100 >100 >100 >100 >100 >100 | >100 >100 >100 >100 >100 >100 >100 >100 | >100 >100 >100 >100 >100 >100 >100 >100 |
| H | Cl | OCH₃ | Br | Br | 37 | >100 | 46 | >100 | >100 | >100 | >100 | >100 |
| H | Cl | OCH₃ | Br | H | 38 | >100 | >100 | >100 | >100 | >100 | >100 | >100 |
| H | Cl | OCH₃ | H | Br | 39 | >100 | >100 | >100 | >100 | >100 | >100 | >100 |
| H | Cl | OCH₃ | H | H | 40 | >100 | >100 | >100 | >100 | >100 | >100 | >100 |
| H | H | OCH₃ | Br | Br | 41 | >100 | >100 | >100 | >100 | >100 | >100 | >100 |
| H | H | OCH₃ | Br | H | 42 | >100 | >100 | >100 | >100 | >100 | >100 | >100 |
| H | H | OCH₃ | H | Br | 43 | >100 | >100 | >100 | >100 | >100 | >100 | >100 |
| H | H | OCH₃ | H | H | 44 | >100 | >100 | >100 | >100 | >100 | >100 | >100 |
| | | | | | Vanco- | 0.60 | 0.67 | 0.52 | >100 | >100 | >100 | >100 |
| | | | | | mycin | | | | | | | |
| | | | | | Dapto- | 9.2 | 0.68 | 1.0 | >100 | >100 | >100 | >100 |
| | | | | | mycin | | | | | | | |
| | | | | | Ampicillin | 6.9 | 0.19 | >100 | >100 | >100 | >100 | >100 |
| | | | | | Rifampicin | 0.13 | <0.05 | 0.23 | 11 | 1.3 | 7.8 | 8.8 |

TABLE 4
Compilation of log(EC₅₀) results and standard deviations for compounds tested in the various bacterial proliferation assays.

| 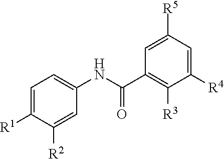 | | | | | Bacterial Proliferation Assay | | | | | | | |
|---|---|---|---|---|---|---|---|---|---|---|---|---|
| Compound Substituents & Substructures | | | | | Compound | C. | S. aureus | | K. | A. | P. | E. |
| R¹ | R² | R³ | R⁴ | R⁵ | #/Name | faecium | Sensitive | Resistant | pneumoniae | baumannii | aeruginosa | cloacae |
| | | | | | Closantel | 0.09 ± 0.09 | −0.33 ± 0.09 | −0.33 ± 0.19 | >2 | >2 | >2 | >2 |
| | | | | | Rafox- | 0.00 ± 0.49 | −0.49 ± 0.13 | −0.46 ± 0.12 | >2 | 1.49 ± 0.40 | >2 | 1.61 ± |
| | | | | | anide | | | | | | | 0.27 |
| 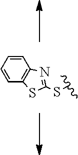 [SMILES: *Sc1nc2ccccc2s1] | Cl Cl Cl Cl H H H H | OH OH OH OH OH OH OH OH | Br Br H H Br Br H H | Br H Br H Br H Br H | 1 2 3 4 5 6 7 8 | −0.29 ± 0.38 1.37 ± 0.41 1.17 ± 0.23 1.80 ± 0.46 −0.05 ± 0.77 1.05 ± 0.68 1.29 ± 0.33 1.83 ± 0.21 | −0.44 ± 0.18 −0.34 ± 0.29 −0.93 ± 0.24 −0.51 ± 0.31 −0.36 ± 0.17 −0.12 ± 0.18 −0.92 ± 0.19 −0.69 ± 0.08 | −0.26 ± 0.32 −0.15 ± 0.28 −0.77 ± 0.42 −0.35 ± 0.24 −0.28 ± 0.07 0.04 ± 0.21 −0.85 ± 0.23 −0.39 ± 0.27 | >2 >2 >2 >2 >2 >2 >2 >2 | 1.82 ± 0.24 0.46 ± 0.12 >2 >2 >2 1.06 ± 0.17 >2 >2 | >2 >2 >2 >2 >2 >2 >2 >2 | >2 >2 >2 >2 >2 >2 >2 >2 |
| H | Cl | OH | Br | Br | 9 | 1.86 ± 0.18 | −0.18 ± 0.16 | 0.04 ± 0.28 | 1.98 ± 0.06 | 1.66 ± 0.11 | >2 | 1.89 ± |
| | | | | | | | | | | | | 0.21 |
| H | Cl | OH | Br | H | 10 | >2 | 0.12 ± 0.03 | 0.30 ± 0.26 | >2 | >2 | >2 | >2 |
| H | Cl | OH | H | Br | 11 | >2 | −0.34 ± 0.02 | −0.27 ± 0.18 | >2 | >2 | >2 | >2 |
| H | Cl | OH | H | H | 12 | >2 | 0.49 ± 0.06 | 0.62 ± 0.06 | >2 | >2 | >2 | >2 |
| H | H | OH | Br | Br | 13 | >2 | 0.09 ± 0.10 | 0.30 ± 0.18 | >2 | >2 | >2 | >2 |
| H | H | OH | Br | H | 14 | >2 | 0.81 ± 0.05 | 1.00 ± 0.17 | >2 | >2 | >2 | >2 |
| H | H | OH | H | Br | 15 | >2 | 0.44 ± 0.05 | 0.61 ± 0.17 | 1.89 ± 0.16 | >2 | >2 | >2 |
| H | H | OH | H | H | 16 | >2 | 1.44 ± 0.09 | 1.71 ± 0.06 | >2 | >2 | >2 | >2 |
| 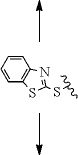 [SMILES: *Sc1nc2ccccc2s1] | Cl Cl Cl H H H | H H H H H H | Br Br (H) H Br Br (H) H | Br H (Br) H Br H (Br) H | 17 18 19 20 21 22 | >2 >2 >2 >2 >2 >2 | >2 >2 1.63 ± 0.19 >2 >2 >2 | >2 >2 >2 >2 >2 >2 | >2 >2 >2 >2 >2 >2 | >2 >2 >2 >2 >2 >2 | >2 >2 >2 >2 >2 >2 | >2 >2 >2 >2 >2 >2 |
| H | Cl | H | Br | Br | 23 | >2 | 1.14 ± 0.12 | 1.07 ± 0.13 | >2 | >2 | >2 | >2 |
| H | Cl | H | Br (H) | H (Br) | 24 | >2 | 1.67 ± 0.06 | 1.83 ± 0.11 | >2 | >2 | >2 | >2 |
| H | Cl | H | H | H | 25 | >2 | >2 | >2 | >2 | >2 | >2 | >2 |
| H | H | H | Br | Br | 26 | >2 | >2 | >2 | >2 | >2 | >2 | >2 |
| H | H | H | Br (H) | H (Br) | 27 | >2 | >2 | >2 | >2 | >2 | >2 | >2 |
| H | H | H | H | H | 28 | >2 | >2 | >2 | >2 | >2 | >2 | >2 |
| 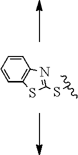 [SMILES: *Sc1nc2ccccc2s1] | Cl Cl Cl Cl H H H H | OCH₃ OCH₃ OCH₃ OCH₃ OCH₃ OCH₃ OCH₃ OCH₃ | Br Br H H Br Br H H | Br H Br H Br H Br H | 29 30 31 32 33 34 35 36 | >2 >2 >2 >2 >2 >2 >2 >2 | >2 >2 >2 >2 >2 >2 >2 >2 | >2 >2 >2 >2 >2 >2 >2 >2 | >2 >2 >2 >2 >2 >2 >2 >2 | >2 >2 >2 >2 >2 >2 >2 >2 | >2 >2 >2 >2 >2 >2 >2 >2 | >2 >2 >2 >2 >2 >2 >2 >2 |
| H | Cl | OCH₃ | Br | Br | 37 | >2 | 1.66 ± 0.17 | >2 | >2 | >2 | >2 | >2 |
| H | Cl | OCH₃ | Br | H | 38 | >2 | >2 | >2 | >2 | >2 | >2 | >2 |
| H | Cl | OCH₃ | H | Br | 39 | >2 | >2 | >2 | >2 | >2 | >2 | >2 |
| H | Cl | OCH₃ | H | H | 40 | >2 | >2 | >2 | >2 | >2 | >2 | >2 |
| H | H | OCH₃ | Br | Br | 41 | >2 | >2 | >2 | >2 | >2 | >2 | >2 |
| H | H | OCH₃ | Br | H | 42 | >2 | >2 | >2 | >2 | >2 | >2 | >2 |
| H | H | OCH₃ | H | Br | 43 | >2 | >2 | >2 | >2 | >2 | >2 | >2 |
| H | H | OCH₃ | H | H | 44 | >2 | >2 | >2 | >2 | >2 | >2 | >2 |
| | | | | | Vanco- | −0.23 ± 0.26 | −0.18 ± 0.25 | −0.29 ± 0.52 | >2 | >2 | >2 | >2 |
| | | | | | mycin | | | | | | | |
| | | | | | Dapto- | 0.97 ± 0.46 | −0.17 ± 0.24 | 0.01 ± 0.08 | >2 | >2 | >2 | >2 |
| | | | | | mycin | | | | | | | |
| | | | | | Ampicillin | 0.84 ± 0.29 | −0.71 ± 0.37 | >2 | >2 | >2 | >2 | >2 |
| | | | | | Rifam- | −0.90 ± 0.49 | <−1.3 | −0.64 ± 0.44 | 1.04 ± 0.06 | 0.12 ± 0.05 | 0.89 ± 0.29 | 0.94 ± |
| | | | | | picin | | | | | | | 0.22 |

### Example 61

Evaluation of Compound Effects on Liver and Kidney Cell Viability.

Evaluation of compound cytotoxicities to THLE-3 liver and HEK 293 kidney cells were performed using Alamar Blue-based viability assays. THLE-3 cells were maintained in Clonetics BEBM medium (Lonza, CC-3171) supplemented with the BEGM bullet kit (Lonza, CC-3170) and 10% FBS. HEK 293 cells were maintained in MEM medium (Corning Cellgro, 10-009 CV) supplemented with 10% FBS (Sigma, F2242). All assays were carried out in 384-well plates (BRAND cell culture grade plates, 781980). Cells at 80% confluence were harvested and diluted in growth medium, then 45 μL of the THLE-3 cells (1,500 cells/well) or HEK 293 cells (1,500 cells/well) were dispensed per well, and plates were sealed with “Breathe Easy” oxygen permeable membranes (Diversified Biotech) and incubated at 37° C., 5% CO₂, for 24 h. The following day, 1 μL of the compound stocks (10 mM to 4.6 μM, 3-fold dilutions in DMSO) were pre-diluted by pin-transfer into 25 μL of the relevant growth mediums. Then, 15 μL aliquots of the diluted compounds were added to the cell assay plates to give inhibitor concentration ranges of 100 μM to 46 nM during the assay (final DMSO concentration of 0.1% was maintained during the assay). Plates were sealed with “Breathe Easy” oxygen permeable membranes and incubated for an additional 48 h at 37° C. and 5% CO₂. The Alamar Blue reporter reagents were then added to a final concentration of 10%, the plates incubated at 37° C. and 5% CO₂, and sample fluorescence (535 nm excitation, 590 nm emission) was read using a Molecular Devices FlexStation II 384-well plate reader (readings taken between 4-24 h of incubation so as to achieve signals in the 30-60% range for conversion of resazurin to resorufin). Cell viability was calculated as per vendor instructions (Thermo Fisher—Alamar Blue cell viability assay manual). Cytotoxicity CC₅₀ values for the test compounds were obtained by plotting the % resazurin reduction results in GraphPad Prism 6 and analyzing by non-linear regression using the log(inhibitor) vs. response (variable slope) equation. Results presented represent the averages of CC₅₀ values obtained from at least triplicate experiments.

### Example 62

While some GroEL/ES inhibitors can target human HSP60/10 in vitro, many display moderate to low cytotoxicity to human cells.

Knowing which compounds were effective GroEL/ES inhibitors with antibacterial properties, we next evaluated whether they would 1) inhibit human HSP60/10, and 2) exhibit cytotoxicity to two cell lines that we typically employ for cell viability testing in vitro: THLE-3 liver cells and HEK 293 kidney cells. The HSP60/10-dMDH folding assay was conducted analogously to the GroEL/ES-dMDH folding assay so IC₅₀ results could be directly compared. The human cell cytotoxicity assays used Alamar Blue reagents to measure the viability of liver and kidney cells that had been incubated with test compounds over a 48 h time period. Biochemical inhibition (IC₅₀) and cell viability (cytotoxicity; CC₅₀) results for these assays are presented in Table 5 and Table 6.

TABLE 5
Compilation of IC₅₀ and CC₅₀ results for compounds tested in the human HSP60/10-dMDH refolding assay and the liver (THLE-3)
and kidney (HEK 293) cell viability assays.

| 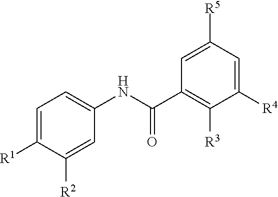 | | | | | | | | |
|---|---|---|---|---|---|---|---|---|
| Compound Substituents & Substructures | | | | | Compound | HSP60/10-dMDH | Human Cell Viability CC₅₀ (μM) | |
| R¹ | R² | R³ | R⁴ | R⁵ | #/Name | Refolding IC₅₀ (μM) | Liver (THLE-3) | Kidney (HEK 293) |
| | | | | | Closantel | 2.5 | 52 | 75 |
| | | | | | Rafoxanide | 2.8 | 24 | >100 |
| 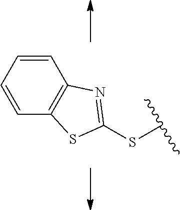 [SMILES: *Sc1nc2ccccc2s1] | Cl Cl Cl Cl H H H H | OH OH OH OH OH OH OH OH | Br Br H H Br Br H H | Br H Br H Br H Br H | 1 2 3 4 5 6 7 8 | 4.2 6.9 >100 >100 4.7 26 >100 >100 | 14 19 14 34 20 36 12 33 | 76 64 45 64 66 71 42 74 |
| H | Cl | OH | Br | Br | 9 | 29 | 9.2 | 18 |
| H | Cl | OH | Br | H | 10 | >100 | 16 | 32 |
| H | Cl | OH | H | Br | 11 | >100 | 4.1 | 15 |
| H | Cl | OH | H | H | 12 | >100 | 16 | 37 |
| H | H | OH | Br | Br | 13 | 63 | 25 | 34 |
| H | H | OH | Br | H | 14 | >100 | 56 | 82 |
| H | H | OH | H | Br | 15 | >100 | 16 | 35 |
| H | H | OH | H | H | 16 | >100 | 68 | >100 |
| 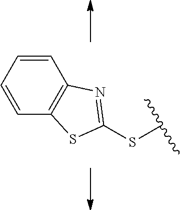 [SMILES: *Sc1nc2ccccc2s1] | Cl Cl Cl H H H | H H H H H H | Br Br (H) H Br Br (H) H | Br H (Br) H Br H (Br) H | 17 18 19 20 21 22 | >100 >100 >100 >100 >100 >100 | >100 88 82 >100 82 >100 | >100 >100 90 >100 74 >100 |
| H | Cl | H | Br | Br | 23 | >100 | 45 | 53 |
| H | Cl | H | Br (H) | H (Br) | 24 | >100 | 50 | 51 |
| H | Cl | H | H | H | 25 | >100 | >100 | >100 |
| H | H | H | Br | Br | 26 | >100 | >100 | >100 |
| H | H | H | Br (H) | H (Br) | 27 | >100 | >100 | >100 |
| H | H | H | H | H | 28 | >100 | >100 | >100 |
| 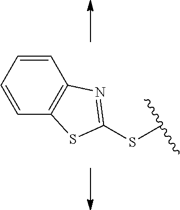 [SMILES: *Sc1nc2ccccc2s1] | Cl Cl Cl Cl H H H H | OCH₃ OCH₃ OCH₃ OCH₃ OCH₃ OCH₃ OCH₃ OCH₃ | Br Br H H Br Br H H | Br H Br H Br H Br H | 29 30 31 32 33 34 35 36 | >100 >100 >100 >100 >100 >100 >100 >100 | >100 >100 >100 >100 >100 >100 >100 >100 | >100 >100 >100 >100 >100 >100 >100 >100 |
| H | Cl | OCH₃ | Br | Br | 37 | >100 | >100 | >100 |
| H | Cl | OCH₃ | Br | H | 38 | >100 | >100 | >100 |
| H | Cl | OCH₃ | H | Br | 39 | >100 | 94 | >100 |
| H | Cl | OCH₃ | H | H | 40 | >100 | >100 | >100 |
| H | H | OCH₃ | Br | Br | 41 | >100 | >100 | >100 |
| H | H | OCH₃ | Br | H | 42 | >100 | >100 | >100 |
| H | H | OCH₃ | H | Br | 43 | >100 | >100 | >100 |
| H | H | OCH₃ | H | H | 44 | >100 | >100 | >100 |
| | | | | | Vancomycin | >100 | >100 | >100 |
| | | | | | Daptomycin | >100 | >100 | >100 |
| | | | | | Ampicillin | >100 | >100 | >100 |
| | | | | | Rifampicin | >100 | 72 | >100 |

TABLE 6
Compilation of log(IC₅₀) and log(CC₅₀) results and standard deviations for compounds tested in human HSP60/10-dMDH refolding
assay and the liver (THLE-3) and kidney (HEK 293) cell viability assays.

| 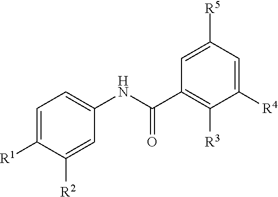 | | | | | | | | |
|---|---|---|---|---|---|---|---|---|
| Compound Substituents & Substructures | | | | | Compound | HSP60/10-dMDH | Human Cell Viability Assay | |
| R¹ | R² | R³ | R⁴ | R⁵ | #/Name | Refolding Assay | Liver (THLE-3) | Kidney (HEK 293) |
| | | | | | Closantel | 0.40 ± 0.30 | 1.72 ± 0.18 | 1.88 ± 0.13 |
| | | | | | Rafoxanide | 0.45 ± 0.39 | 1.38 ± 0.10 | >2 |
| 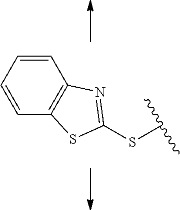 [SMILES: *Sc1nc2ccccc2s1] | Cl Cl Cl Cl H H H H | OH OH OH OH OH OH OH OH | Br Br H H Br Br H H | Br H Br H Br H Br H | 1 2 3 4 5 6 7 8 | 0.62 ± 0.34 0.84 ± 0.24 >2 >2 0.67 ± 0.23 1.41 ± 0.34 >2 >2 | 1.15 ± 0.18 1.28 ± 0.21 1.15 ± 0.21 1.53 ± 0.13 1.30 ± 0.21 1.56 ± 0.19 1.08 ± 0.14 1.52 ± 0.17 | 1.88 ± 0.17 1.81 ± 0.25 1.65 ± 0.24 1.81 ± 0.21 1.82 ± 0.17 1.85 ± 0.27 1.62 ± 0.39 1.87 ± 0.20 |
| H | Cl | OH | Br | Br | 9 | 1.46 ± 0.14 | 0.96 ± 0.27 | 1.26 ± 0.39 |
| H | Cl | OH | Br | H | 10 | >2 | 1.20 ± 0.11 | 1.51 ± 0.40 |
| H | Cl | OH | H | Br | 11 | >2 | 0.61 ± 0.19 | 1.18 ± 0.74 |
| H | Cl | OH | H | H | 12 | >2 | 1.20 ± 0.13 | 1.57 ± 0.45 |
| H | H | OH | Br | Br | 13 | 1.80 ± 0.15 | 1.40 ± 0.11 | 1.53 ± 0.27 |
| H | H | OH | Br | H | 14 | >2 | 1.75 ± 0.10 | 1.91 ± 0.14 |
| H | H | OH | H | Br | 15 | >2 | 1.20 ± 0.07 | 1.54 ± 0.42 |
| H | H | OH | H | H | 16 | >2 | 1.83 ± 0.06 | >2 |
| 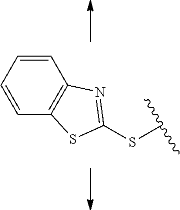 [SMILES: *Sc1nc2ccccc2s1] | Cl Cl Cl H H H | H H H H H H | Br Br (H) H Br Br (H) H | Br H (Br) H Br H (Br) H | 17 18 19 20 21 22 | >2 >2 >2 >2 >2 >2 | >2 1.94 ± 0.08 1.91 ± 0.11 >2 1.91 ± 0.19 >2 | >2 >2 1.95 ± 0.07 >2 1.87 ± 0.14 >2 |
| H | Cl | H | Br | Br | 23 | >2 | 1.65 ± 0.14 | 1.73 ± 0.08 |
| H | Cl | H | Br (H) | H (Br) | 24 | >2 | 1.70 ± 0.09 | 1.71 ± 0.15 |
| H | Cl | H | H | H | 25 | >2 | >2 | >2 |
| H | H | H | Br | Br | 26 | >2 | >2 | >2 |
| H | H | H | Br (H) | H (Br) | 27 | >2 | >2 | >2 |
| H | H | H | H | H | 28 | >2 | >2 | >2 |
| 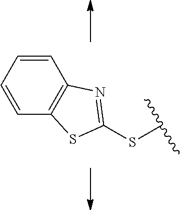 [SMILES: *Sc1nc2ccccc2s1] | Cl Cl Cl Cl H H H H | OCH₃ OCH₃ OCH₃ OCH₃ OCH₃ OCH₃ OCH₃ OCH₃ | Br Br H H Br Br H H | Br H Br H Br H Br H | 29 30 31 32 33 34 35 36 | >2 >2 >2 >2 >2 >2 >2 >2 | >2 >2 >2 >2 >2 >2 >2 >2 | >2 >2 >2 >2 >2 >2 >2 >2 |
| H | Cl | OCH₃ | Br | Br | 37 | >2 | >2 | >2 |
| H | Cl | OCH₃ | Br | H | 38 | >2 | >2 | >2 |
| H | Cl | OCH₃ | H | Br | 39 | >2 | 1.97 ± 0.16 | >2 |
| H | Cl | OCH₃ | H | H | 40 | >2 | >2 | >2 |
| H | H | OCH₃ | Br | Br | 41 | >2 | >2 | >2 |
| H | H | OCH₃ | Br | H | 42 | >2 | >2 | >2 |
| H | H | OCH₃ | H | Br | 43 | >2 | >2 | >2 |
| H | H | OCH₃ | H | H | 44 | >2 | >2 | >2 |
| | | | | | Vancomycin | >2 | >2 | >2 |
| | | | | | Daptomycin | >2 | >2 | >2 |
| | | | | | Ampicillin | >2 | >2 | >2 |
| | | | | | Rifampicin | >2 | 1.86 ± 0.2 | >2 |

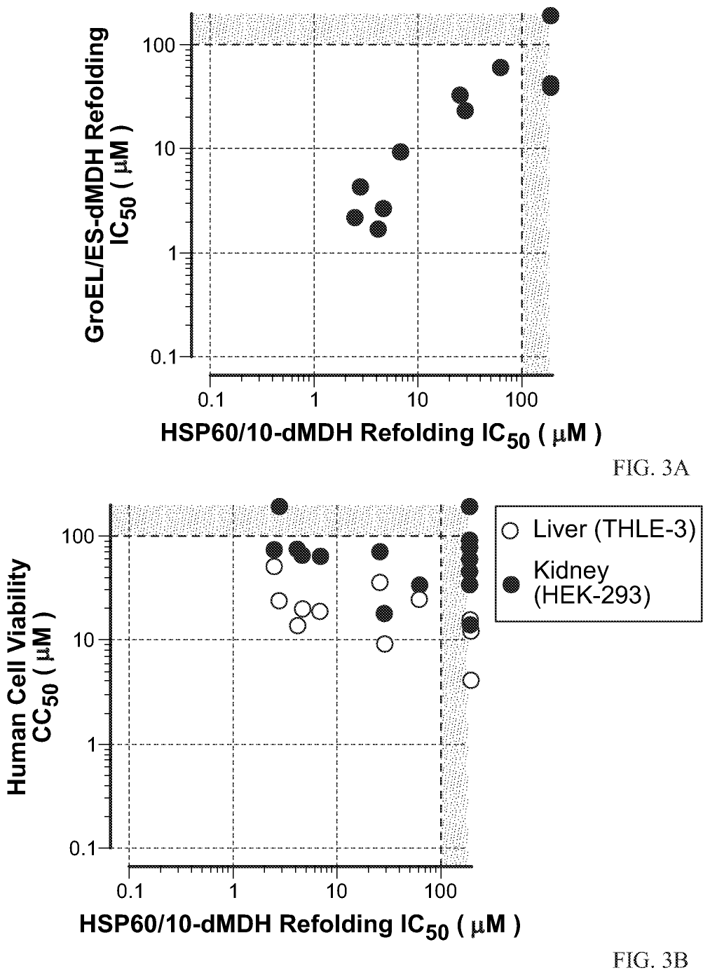

While some analogs selectively inhibit E. coli GroEUES over human HSP60/10 (e.g. 3, 4, 7, and 8), IC₅₀ values between the GroEL/ES-dMDH and HSP60/10-dMDH folding assays were nearly the same for most analogs (FIG. 3A—Spearman correlation coefficient comparing log(IC₅₀) values in each assay is 0.8351, p<0.0001). We note, though, that comparison of these results is convoluted by the fact that some of these analogs also inhibit native MDH, and thus could be false positives in the HSP60/10-dMDH folding assay owing to simply inhibiting the MDH reporter reaction. While HSP60/10 inhibition could potentially be teased out by employing rhodanese as the denatured reporter enzyme as we do in the case of the GroEL/ES-dRho refolding assay, in our experience, the equivalent HSP60/10-dRho refolding is not as robust, potentially owing to the lower stability of human HSP60 compared to E. coli GroEL.

When comparing the biochemical and cell-based results, there does not appear to be a noticeable correlation between HSP60/10-dMDH refolding assay IC₅₀ values and liver and kidney cell viability assay CC₅₀ values (FIG. 3B—(Spearman correlation coefficient values are 0.4791 (p<0.0008) and 0.3286 (p<0.0258) when comparing HSP60/10-dMDH refolding assay log(IC₅₀) values with liver and kidney cell viability log(CC₅₀) values, respectively). Interestingly, compounds that bear the R¹ benzothiazole and R³ hydroxyl substructures (1-8) are generally less cytotoxic against the HEK 293 cells than their counterparts without the R¹ benzothiazole (9-16), and also less cytotoxic to the THLE-3 liver cells. Since inclusion of these two substructures generally afforded more potent chaperonin inhibitors, the differences between these results may suggest that compound cytotoxicities are predominantly a result of off-target effects and not from targeting HSP60/10 itself. When comparing EC₅₀ values of compounds inhibiting the proliferation of susceptible and methicillin-resistant S. aureus with cell viability CC₅₀ values against the human liver and kidney cells (FIG. 3C), we note that many analogs exhibit >50-fold selectivity indices.

### Example 63

Evaluation of MRSA Resistance Generation Against Lead Inhibitors.

To identify potential resistance toward compounds 1 and 11, a liquid culture, 12-day serial passage assay was employed as per previously reported procedures, and using the ATCC BAA-44 MRSA strain. MRSA bacteria were streaked onto a Tryptic Soy agar plate and grown overnight at 37° C. A fresh aliquot of Tryptic Soy Broth (TSB) was inoculated with a single bacterial colony and the cultures were grown overnight at 37° C. with shaking (250 rpm). The overnight culture was then sub-cultured (1:5 dilution) into a fresh aliquot of media and grown at 37° C. for 1 h with shaking, then diluted into fresh media to achieve a final OD₆₀₀ reading of 0.01. Aliquots of the diluted culture (200 μL) were dispensed to 96 well plates along with addition of 2 μL of test compounds in DMSO (1, 11, vancomycin, and a previously-reported GroEUES inhibitor, “28R”). The inhibitor concentration range during the resistance assay was 100 μM to 48.8 nM (2-fold dilution series). Plates were sealed with “Breathe Easy” oxygen permeable membranes (Diversified Biotech) and left to incubate at 37° C. without shaking (stagnant assay). OD₆₀₀ readings were taken at the 24 h time point to monitor for bacterial growth. A second set of baseline control plates were prepared analogously, without any bacteria added, to correct for possible compound absorbance and/or precipitation, as well as plate and media baseline effects. For inoculations on subsequent days, bacteria from the wells with the highest drug concentration where the OD₆₀₀ was >0.2 were diluted with fresh media to OD₆₀₀ of 0.01 and dispensed into a new 96-well plate. Test compounds were added, and the bacteria propagated again as described above. This procedure was repeated each day for a total of 12 days to observe changes in EC₅₀ values over each passage. EC₅₀ values for the test compounds were obtained by plotting the OD₆₀₀ results from each passage in GraphPad Prism 6 and analyzing by non-linear regression using the log(inhibitor) vs. response (variable slope) equation. Results presented represent the averages of EC₅₀ values obtained from triplicate experiments.

### Example 64

MRSA Cannot Readily Generate Acute Resistance to Lead Analogs.

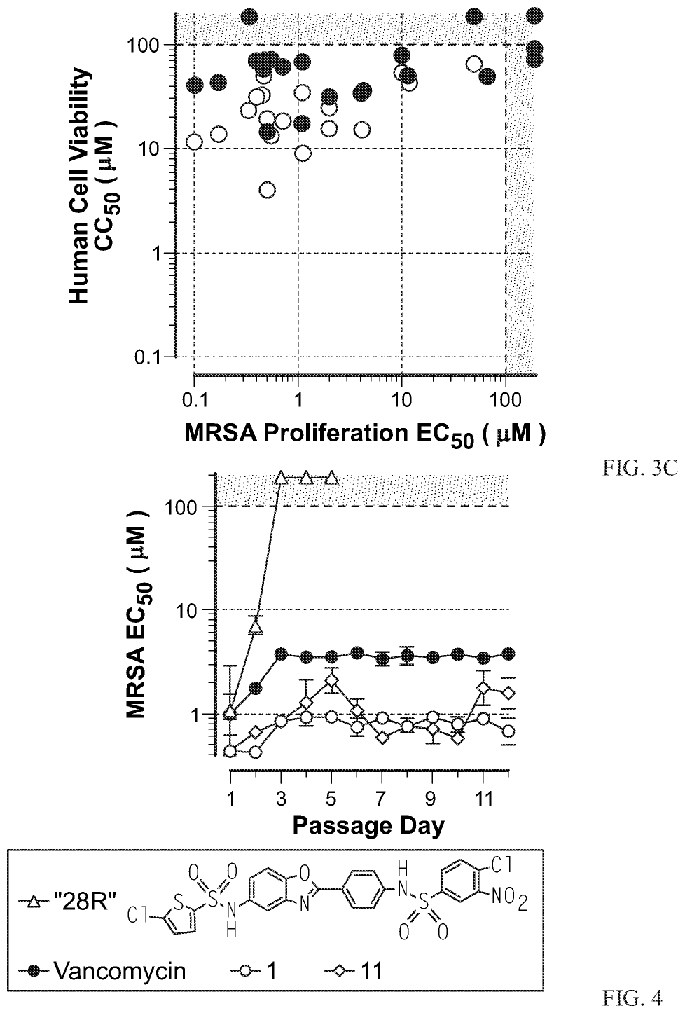

After identifying which compounds selectively inhibited the GroEL chaperonin system and killed bacteria, we next evaluated whether bacteria could quickly develop resistance to lead candidate inhibitors. This was a concern we encountered with another series of GroEL/ES inhibitors we have been studying, represented by the bis-sulfonamido compound “28R” shown in FIG. 4. For this experiment, we adapted a liquid culture resistance assay from the previously established procedures of Kim et al., and used our MRSA strain as the test bacteria. Test compounds were incubated in dilution series with MRSA for 24 h and EC₅₀ values were determined. The first well exhibiting OD₆₀₀ readings >0.2 were then sub-cultured for another 24 h with test compound again in dilution series. Serial passage in this manner was conducted for a total of 12 days, and each day EC₅₀ values for the test compounds were determined: inhibitors to which MRSA could rapidly generate resistance would exhibit increasing EC₅₀ results over each successive passage. Rapid resistance was observed for the previously reported bis-sulfonamide compound, 28R, but was found to be reversible and likely owing to increased inhibitor efflux. We evaluated two of our lead GroEL inhibitors, 1 and 11, along with vancomycin, and found that all three exhibited exemplary antibiotic efficacy to which this MRSA strain was not able to easily generate resistance.

### Example 65

Control Compounds for Assays.

For all of the biochemical assays (GroEL/ES and HSP60/10-mediated dMDH and dRho refolding assays, and native MDH and Rho enzymatic activity counter-screens), DMSO was used as a negative control, and a panel of our previously discovered and reported chaperonin inhibitors were used as positive controls: e.g,. compound 1 herein; compounds 9 and 18 from Johnson et. al 2014 and Abdeen et. al 2016; suramin and compound 2h-p from Abdeen et. al 2016; and compounds 20R, 20L, and 28R from Abdeen et. al 2018 the contents of which are incorporated herein by reference. For the bacterial proliferation assays, control compounds included the aforementioned panel of previously reported chaperonin inhibitors as well as vancomycin, daptomycin, ampicillin, and rifampicin. For the human cell viability assays, control compounds include the aforementioned compounds as well as other protein homeostasis inhibitors, such as bortezomib (proteasome inhibitor); VER-155008 (HSP70 inhibitor); and ganetespib and 17-DMAG (HSP90 inhibitors).

### Example 66

S. aureus Biofilm Prevention Assay.

The biofilm prevention assay was carried out with S. aureus Rosenbach (ATCC 25923) using a quantitative crystal violet-based adherence assay on 96-well plates. S. aureus (ATCC 25923) bacteria were streaked onto a Tryptic Soy Broth (TSB) agar plate and grown overnight at 37° C. A fresh aliquot of TSB media was inoculated with a single bacterial colony and the cultures were grown overnight at 37° C. with shaking (250 rpm). The overnight culture was then sub-cultured (1:5 dilution) into a fresh aliquot of TSB media supplemented to a final concentration of 0.5% glucose and grown at 37° C. for 1 h with shaking, then diluted into fresh TSB media supplemented with 0.5% glucose to achieve a final OD₆₀₀ reading of 0.01. Aliquots of the diluted culture (100 μL) were dispensed to 96 well polystyrene plates along with addition of 1 μL test compounds in DMSO. The inhibitor concentration range during the assay was 100 μM to 46 nM (3-fold dilution series). A second set of baseline control plates were prepared analogously, but without any bacteria added, to correct for possible compound absorbance and/or precipitation. Plates were sealed with “Breathe Easy” oxygen permeable membranes (Diversified Biotech) and left to incubate at 37° C. without shaking (stagnant assay) until the biofilm was formed. After 24 h, the planktonic cultures were removed and the plates were washed gently 2-3 times with 200 μl of water. Next, the plates were air dried and the adherent biofilms were stained with 150 μL of crystal violet solution (2.3% crystal violet in 20% Ethanol, Sigma Aldrich #HT90132) for 15 minutes at R.T. The unbound crystal violet stain was removed, then plates were gently washed again with running water and air dried for 10 min. Quantitative assessment of biofilm formation was obtained by adding 100 μL of developer solution (4:1:5 mixture of MeOH:AcOH:H₂O) per well. Well absorbance was then read at 595 nm using a Molecular Devices SpectraMax Plus384 microplate reader. EC₅₀ values for the test compounds were obtained by plotting the A₅₉₅ nm results in GraphPad Prism 6 and analyzing by non-linear regression using the log(inhibitor) vs. response (variable slope) equation. Results presented represent the averages of EC₅₀ values obtained from at least triplicate experiments.

### Example 67

S. aureus Biofilm Penetration and Bactericidal Activity Assay.

The biofilm penetration and bactericidal activity assay was carried out with S. aureus Rosenbach (ATCC 25923) as described previously by Kwasny et al. S. aureus bacteria were streaked onto a Tryptic Soy Broth (TSB) agar plate and grown overnight at 37° C. A fresh aliquot of TSB media was inoculated with a single bacterial colony and the cultures were grown overnight at 37° C. with shaking (250 rpm). The overnight culture was then sub-cultured (1:5 dilution) into a fresh aliquot of TSB media supplemented with 0.5% glucose and grown at 37° C. for 1 h with shaking, then diluted into fresh TSB media supplemented with 0.5% glucose to achieve a final OD₆₀₀ reading of 0.01. Aliquots of the diluted culture (100 μL) were dispensed to 96 well polystyrene plates without any compounds added. A second set of control plates were prepared analogously, but without any bacteria added. Plates were sealed with “Breathe Easy” oxygen permeable membranes (Diversified Biotech) and left to incubate at 37° C. without shaking (stagnant assay) until biofilm was formed. After 24 h, the planktonic cultures (or media blanks in the control plates) were removed and the plates were washed gently 3 times with 200 μL of sterile phosphate buffered saline (PBS). Then, 100 μL aliquots of fresh TSB media were dispensed to the plates along with addition of 1 μL of test compounds in DMSO. The inhibitor concentration range during the assay was 100 μM to 46 nM (3-fold dilution series). The plates were sealed with “Breathe Easy” membranes and incubated at 37° C. without shaking to allow compounds to penetrate and kill bacteria in the biofilms. After 24 h, the cultures were removed and plates were washed again gently 3 times with 200 μL of sterile PBS. The remaining bacteria in the biofilms were allowed to recover by adding 100 μL of fresh TSB media per well and incubating for 24 h at 37° C. At the end of this final incubation, bacterial growth was monitored by measuring the OD_{600 nm} using a Molecular Devices SpectraMax Plus384 microplate reader. EC₅₀ values for the test compounds were obtained by plotting the OD_{600 nm} results in GraphPad Prism 6 and analyzing by non-linear regression using the log(inhibitor) vs. response (variable slope) equation. Results presented represent the averages of EC₅₀ values obtained from at least triplicate experiments.

### Example 68

Compound 1 is Bactericidal to S. aureus within Established Biofilms

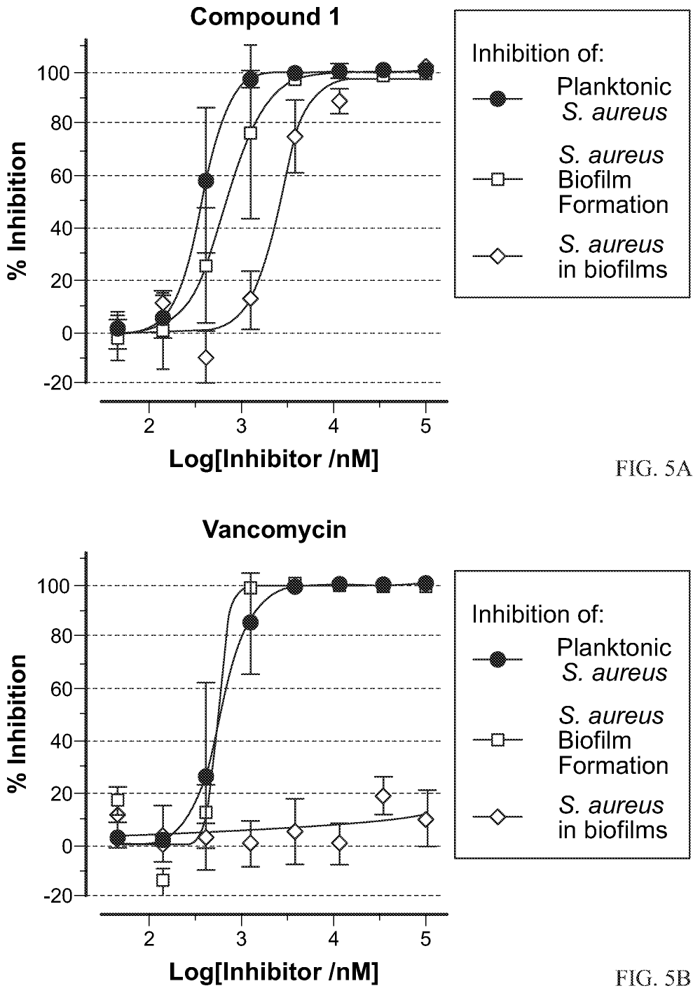

While we found that S. aureus is not able to minimally easily generated resistance to compounds 1 and 11, what remained to be seen was whether this series of inhibitors would be effective at preventing bacteria from establishing biofilms and killing bacteria within already established biofilms. Establishing biofilms is another effective mechanism by which S. aureus can evade the effects of many current antibiotics, including vancomycin. To gauge the efficacy of lead inhibitors at preventing S. aureus from forming biofilms, we employed an assay similar to the liquid culture assay we used to determine inhibition of bacterial proliferation, with a few modifications. Briefly, test compounds (1, 2, 5, 8, 11, and vancomycin) were incubated with S. aureus bacteria in media supplemented with 0.5% glucose (to support biofilm formation) for 24 h at 37° C. After 24 h, the supernatant was removed, the wells were gently washed, and the biofilm that had formed on the well surfaces were stained with crystal-violet and quantified by UV-Vis spectroscopy. We found that all of the compound 1 analogs tested, and vancomycin, were able to prevent S. aureus from forming biofilms with EC₅₀ values nearly equipotent to antibacterial EC₅₀s we determined against planktonic bacterial growth. Representative dose-response curves for compound 1 and vancomycin tested in these assays are presented in FIG. 5, with a tabulation of test compound EC₅₀ results presented in Table 7.

TABLE 7
Compilation of EC₅₀ values for inhibitors tested in the biofilm formation and penetration/bactericidal acitivity assays.

| 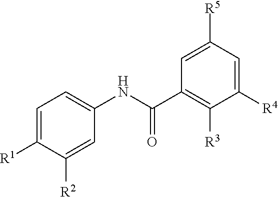 | | | | | S. aureus Proliferation & Biofilm Assay EC₅₀ (μM) | | | |
|---|---|---|---|---|---|---|---|---|
| Compound Substituents & Substructures | | | | | Compound | Planktonic | Preventing Biofilm | Killing Bacteria in |
| R¹ | R² | R³ | R⁴ | R⁵ | #/Name | Growth | Formation | Biofilms |
| 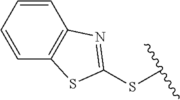 [SMILES: *Sc1nc2ccccc2s1] | Cl Cl H H | OH OH OH OH | Br Br Br H | Br H Br H | 1 2 5 8 | 0.36 0.45 0.44 0.20 | 0.72 1.0 0.54 0.91 | 2.4 5.6 2.3 2.0 |
| H | Cl | OH | H | Br | 11 | 0.46 | 0.44 | 6.9 |
| | | | | | Vancomycin | 0.67 | 0.54 | >100 |

Next, we evaluated whether or not compounds would be bactericidal to S. aureus that were within already established biofilms. In this assay, we first grew S. aureus bacteria for 24 h in the absence of test compounds so that they could establish biofilms in the wells. After 24 h, the cultures were removed, the wells were washed gently, and fresh media was added along with test compounds or vancomycin. The cultures were incubated in the presence of test compounds for another 24 h, then the wells were gently washed again to remove compounds and any planktonic bacteria that had emerged. Fresh media was then added and the cultures were incubated for another 24 h to allow any viable bacteria remaining in the biofilms to emerge and grow planktonically again. While there were 5-15-fold shifts in EC₅₀ values for the compound 1 analogs killing planktonic vs. biofilm bacteria (FIG. 5 and Table 7), these are surprising results considering vancomycin was completely ineffective against biofilm bacteria (EC₅₀>100 μM).

### Example 68

Calculation of IC₅₀/EC₅₀/CC₅₀ values and statistical considerations.

All IC₅₀/EC₅₀/CC₅₀ results reported are averages of values determined from individual dose-response curves in assay replicates as follows: 1) Individual I/E/CC₅₀ values from assay replicates were first log-transformed and the average log(I/E/CC₅₀) values and standard deviations (SD) calculated; 2) Replicate log(I/E/CC₅₀) values were evaluated for outliers using the ROUT method in GraphPad Prism 6 (Q of 10%); and 3) Average I/E/CC₅₀ values were then back-calculated from the average log(I/E/CC₅₀) values. To compare log(I/E/CC₅₀) values between different assays, two-tailed Spearman correlation analyses were performed using GraphPad Prism 6 (95% confidence level). For compounds where log(I/E/CC₅₀) values were greater than the maximum compound concentrations tested (i.e. >2.0, or >100 μM), results were represented as 0.1 log units higher than the maximum concentrations tested (i.e. 2.1, or 126 μM), so as not to overly bias comparisons because of the unavailability of definitive values for these inactive compounds.

1. A compound of the formula I 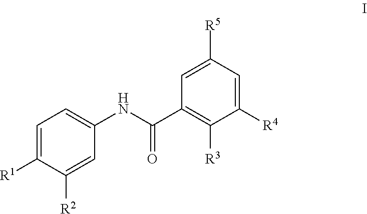
wherein
R¹ is H or 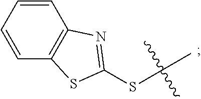 [SMILES: *Sc1nc2ccccc2s1]
R² is a halogen or H;
R³ is H, OH, or OCH₃;
R⁴ is a halogen or H; and
R⁵ is a halogen or H;
provided that at least one of R¹, R², R³, R⁴, and R⁵ is not H;
or a pharmaceutically acceptable salt thereof.

2. The compound or pharmaceutically acceptable salt of claim 1, having the formula 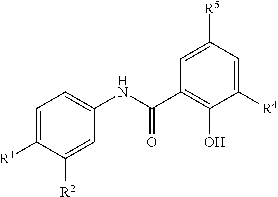
or a pharmaceutically acceptable salt thereof.

3. The compound or pharmaceutically acceptable salt of claim 1, wherein R¹ is H.

4. The compound or pharmaceutically acceptable salt of claim 1, wherein R¹ is

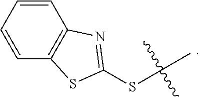 [SMILES: *Sc1nc2ccccc2s1]

5. The compound or pharmaceutically acceptable salt of claim 1, wherein R² is H.

6. The compound or pharmaceutically acceptable salt of claim 1, wherein R² is Cl.

7. The compound or pharmaceutically acceptable salt of claim 1, wherein R⁴ is H.

8. The compound or pharmaceutically acceptable salt of claim 1, wherein R⁴ is Br.

9. The compound or pharmaceutically acceptable salt of claim 1, wherein R⁵ is H.

10. The compound or pharmaceutically acceptable salt of claim 1, wherein R⁵ is Br.

11. The compound or pharmaceutically acceptable salt of claim 1, wherein R⁴ is Br and R⁵ is Br.

12. The compound or pharmaceutically acceptable salt of claim 11, wherein R¹ is

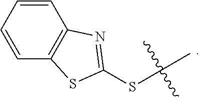 [SMILES: *Sc1nc2ccccc2s1]

13. The compound or pharmaceutically acceptable salt of claim 12, wherein R² is Cl.

14. The compound or pharmaceutically acceptable salt of claim 12, wherein R² is H.

15. The compound or pharmaceutically acceptable salt of claim 1, having the formula 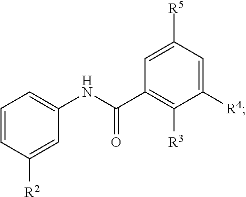
or a pharmaceutically acceptable salt thereof.

16. The compound or pharmaceutically acceptable salt of claim 1, wherein at least two of R¹, R², R³, R⁴, and R⁵ are independently not H.

17. The compound or pharmaceutically acceptable salt of claim 1, wherein at least three of R¹, R², R³, R⁴, and R⁵ are independently not H.

18. The compound or pharmaceutically acceptable salt of claim 1, wherein at least four of R¹, R², R³, R⁴, and R⁵ are independently not H.

19. A method of killing or inhibiting the growth of bacteria comprising: contacting the bacteria with a compound of formula I 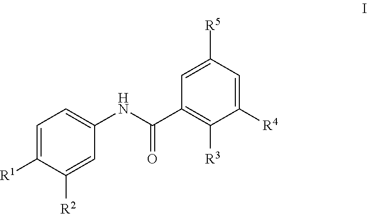
wherein
R¹ is H or 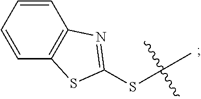 [SMILES: *Sc1nc2ccccc2s1]
R² is a halogen or H;
R³ is H, OH, or OCH₃;
R⁴ is a halogen or H; and
R⁵ is a halogen or H;
provided that at least one of R¹, R², R³, R⁴, or R⁵ is not H;
or a pharmaceutically acceptable salt thereof.
to bacteria.

20. The method of claim 19, having the formula 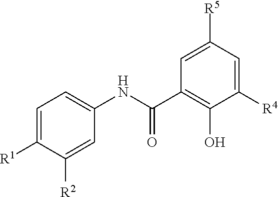
or a pharmaceutically acceptable salt thereof.

21.-37. (canceled)
